(19) United States
(12) Patent Application Publication
(10) Pub. No.: US 2026/0091086 A1
(43) Pub. Date: Apr. 2, 2026

(54) POLYPEPTIDE COMBINATIONS AND USES THEREOF

(71) Applicant: MEDIMMUNE LIMITED, Cambridge (GB)

(72) Inventor: Sarah WILL, Wilmington, DE (US)

(21) Appl. No.: 19/212,361

(22) Filed: May 19, 2025

Related U.S. Application Data
(60) Provisional application No. 63/649,789, filed on May 20, 2024.
(60) Provisional application No. 63/757,683, filed on Feb. 12, 2025.
(60) Provisional application No. 63/796,820, filed on Apr. 29, 2025.

(51) Int. Cl. A61K 38/26; A61K 38/22; A61P 3/04; C07K 14/575; C07K 14/605

(52) U.S. Cl. CPC A61K 38/26; A61K 38/22; A61P 3/04; C07K 14/575; C07K 14/605; C07K 2319/00

(57) ABSTRACT

The present invention relates to a combination of polypeptides and uses thereof. In particular, the present invention relates to a combination of an amylin receptor agonist and a GLP-1 receptor agonist, and uses thereof.

This application includes a Sequence Listing submitted electronically via EFS-Web (Name: 2025 May 19-Sequence-Listing-2943-3060003; Size: 82,505 bytes; Date of Creation: May 16, 2025), which is hereby incorporated by reference in its entirety.

### FIELD OF THE INVENTION

The present invention relates to a combination of polypeptides and uses thereof. In particular, the present invention relates to a combination of an amylin receptor agonist and a GLP-1 receptor agonist, and uses thereof.

### BACKGROUND

The incidence of obesity and diabetes have been rising in epidemic proportions. Diabetes is characterized by high levels of blood glucose resulting from defects in insulin production, insulin action, or both. Type 2 diabetes mellitus (T2DM) accounts for some 90 to 95 percent of all diagnosed cases of diabetes, and the risk of type 2 diabetes rises with increasing body weight. The prevalence of type 2 diabetes is three to seven times higher in those who are affected by obesity than in normal weight adults, and is 20 times more likely in those with a body mass index (BMI) greater than 35 kg/m². However, weight-loss can improve control or cure type 2 diabetes.

Glucagon and glucagon-like peptide-1 (GLP-1) derive from pre-proglucagon, a 158 amino acid precursor polypeptide that is processed in different tissues to form a number of different proglucagon-derived peptides, including glucagon, glucagon-like peptide-1 (GLP-1), glucagon-like peptide-2 (GLP-2) and oxyntomodulin (OXM), that are involved in a wide variety of physiological functions, including glucose homeostasis, insulin secretion, gastric emptying, and intestinal growth, as well as the regulation of food intake. Glucagon is a 29-amino acid peptide that corresponds to amino acids 33 through 61 of proglucagon (53 to 81 of preproglucagon), while GLP-1 is produced as a 37-amino acid peptide that corresponds to amino acids 72 through 108 of proglucagon (92 to 128 of preproglucagon). GLP-1 (7-36) amide or GLP-1 (7-37) acid are biologically active forms of GLP-1, that demonstrate essentially equivalent activity at the GLP-1 receptor (GLP-1R).

Glucagon is produced by the pancreas and interacts with the glucagon receptor (“GCGR”). Glucagon acts in the liver to raise blood glucose via gluconeogenesis and glycogenolysis. When blood glucose begins to fall, glucagon signals the liver to break down glycogen and release glucose, causing blood glucose levels to rise toward a normal level.

GLP-1 has different biological activities compared to glucagon. It is secreted from gut L-cells and binds to the GLP-1R. Its activities include stimulation of insulin secretion, inhibition of glucagon secretion, and inhibition of food intake.

Both glucagon and GLP-1, acting as agonists at their respective receptors, have been shown to be effective in weight loss. Certain GLP-1 analogues are being sold or are in development for treatment of obesity including, e.g., Liraglutide (Saxenda® from Novo Nordisk) and Semaglutide (Wegovy® from Novo Nordisk). Glucagon/GLP-1 dual agonist peptides such as cotadutide are also known and are in clinical development for treatment of diabetes, obesity, and metabolic dysfunction associated steatohepatitis (MASH).

Amylin analogues are also being considered for the treatment of obesity, excess food intake, and diabetes (see e.g., WO 2018/046719). Pramlintide, a synthetic analogue of human amylin, is clinically used in amylin replacement therapies and simulates the important glucoregulatory actions of amylin. These glucoregulatory actions complement those of insulin by regulating the rate of appearance of glucose in the circulation, and are achieved through three primary mechanisms: slowing the rate of gastric emptying, suppression of post-meal glucagon secretion and suppression of food intake (Roth J D et. al. GLP-1R and amylin agonism in metabolic disease: complementary mechanisms and future opportunities. Br J Pharmacol. 2012; 166 (1): 121-136). Pramlintide has been used as an adjunct to insulin in patients with diabetes who have failed to reach desired glucose control despite optimal insulin therapy (Pullman J, et. al. Pramlintide is used in the management of insulin-using patients with type 2 and type 1 diabetes. Vasc Health Risk Manag. 2006; 2 (3): 203-212). Pramlintide analogues conjugated to lipids to extend their half-life are also known.

Existing agents are not without their problems. GLP-1 agonists, glucagon/GLP-1 agonists and some amylin analogues are reported to be associated with poor tolerability, as nausea and vomiting are common side effects. In addition, GLP-1 agonists and glucagon/GLP-1 agonists are known to result in the loss of lean muscle mass, which is clinically undesirable.

Accordingly, there remains a need for therapeutics that can improve glycemic control, reduce weight, treat type 2 diabetes mellitus (T2DM), and/or treat NASH, while minimizing burdens associated with a reduction in lean muscle mass. It is an object of the present invention to address one or more of these issues.

### SUMMARY OF THE INVENTION

The present inventors have demonstrated combining a particular class of amylin receptor (AMYR) agonists with GLP-1 receptor (GLP-1R) agonists provides advantageous properties. In particular, as exemplified herein, combining AMYR agonists which has selectivity for AMYR as compared with calcitonin receptor (CTR) with GLP-1R agonists results in additional fat-mass specific weight loss and without increased aversion in pre-clinical rat models. Accordingly, the present inventors provide for the first time a combination AMYR agonist and GLP1-R agonist treatment which promotes fat mass loss and preserves lean mass without compromising tolerability, making this a clinically attractive treatment paradigm. As further demonstrated herein, this combination treatment has the potential to provide synergistic effects beyond those observed using AMYR agonists and GLP1-R agonists as monotherapies.

Accordingly, the present invention provides a method for treating and/or preventing a disease or disorder in a subject, the method comprising administering to the subject (a) an amylin receptor (AMYR) agonist, wherein the AMYR agonist has selectivity to AMYR as compared to a calcitonin receptor (CTR); and (b) a GLP-1 receptor (GLP-1R) agonist.

The GLP-1R agonist may activate both a GLP-1R and a glucagon receptor (GCGR).

The method may further comprise administering to the subject a glucagon receptor (GCGR) agonist.

The AMYR agonist, GLP-1R agonist, and/or GCGR agonist may each independently be selected from a polypeptide, small molecule drug, antibody, antibody-drug conjugate, or aptamer; or a pharmaceutically acceptable salt thereof.

The AMYR agonist, GLP-1R agonist, and/or GCGR agonist may each be a polypeptide, or a pharmaceutically acceptable salt thereof.

The AMYR may be a human AMYR, GLP-1R may be a human GLP-1R, and/or the GCGR may be a human GCGR.

The AMYR may be AMY1R, AMY2R and/or AMY3R.

The AMYR agonist may have at least a 2-fold, at least 3-fold, at least 4-fold, at least 5-fold, at least 6-fold, at least 7-fold, at least 8-fold, at least 9-fold, at least 10-fold, at least 12-fold, at least 15-fold, at least 17-fold, at least 20-fold, or at least 25-fold selectivity to AMYR as compared to CTR; optionally wherein the AMYR agonist has at least a 10-fold selectivity to AMYR as compared to CTR.

The GLP-1R agonist may activate both GLP-1R and GCGR. Optionally the GLP-1R agonist may be selective to GLP-1R as compared to GCGR. The GLP-1R agonist may activate GLP-1R and GCGR equally. The GLP-1R agonist may have at least a 1.5-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, at least 17-fold, at least 20-fold, or at least 25-fold selectivity to GLP-1R as compared to GCGR; optionally wherein the GLP-1R agonist has at least a 1.5-fold selectivity to GLP-1R as compared to GCGR.

The AMYR agonist may be an AMYR agonist polypeptide, or pharmaceutically acceptable salt thereof, which comprises or consists of an amino acid sequence having at least 90% identity to pramlintide (KCNTATCATQRLANFLVHSSNNFGPILPPTNVGSNTY-amide, SEQ ID NO: 5).

The AMYR agonist polypeptide may be lipidated, optionally the lipid is selected from the group consisting of C12diacid, C14diacid, C16diacid, C17diacid, C18diacid, C19diacid or C20diacid, optionally wherein the lipid is octadecanedioic acid (C18diacid); and/or C20diacid.

The lipid may be attached to an amino acid residue in the AMYR agonist polypeptide by a linker, optionally wherein the linker is γE, γE-γE, ((O2Oc)-(O2Oc)-γE), ((O2Oc)-(O2Oc)-γEγE), or ((PEG2)-(PEG2)-γE).

The lysine at position 1 of the AMYR agonist polypeptide, or pharmaceutically acceptable salt thereof, may be lipidated; optionally wherein the lysine at position 1 of the AMYR agonist polypeptide is lipidated, the lipid is linked to the lysine via a γE-γE linker, and the lipid is octadecanedioic acid (C18diacid) or icosanedioic acid (C20diacid), preferably octadecanedioic acid (C18diacid).

The AMYR agonist polypeptide, or pharmaceutically acceptable salt thereof, may comprise or consist of an amino acid sequence having at least 90%, at least 95%, at least 97%, at least 99%, or 100% identity to an amino acid sequence selected from the group consisting of.

(SEQ ID NO: 6)
C18diacid-γE-[CNTATC]ATQRLAEFLRHSSNN(αMePhe)GPILPPTNVGSNTY-amide;
(SEQ ID NO: 7)
K(γE-γE-C18diacid)[CNTATC]ATQRLAEFLRHSSNN(αMePhe)GPILPPTNVGSNTY-amide;
(SEQ ID NO: 8)
K(γE-C18diacid)K[CNTATC]ATQRLAEFLRHSSNN(αMePhe)GPILPPTNVGSNTY-amide;
(SEQ ID NO: 9)
K(γE-C18diacid)K[CNTATC]ATQRLANFLRHSSNN(αMePhe)GPILPPTEVGSNTY-amide;
(SEQ ID NO: 10)
K(O2Oc-O2Oc-γE-18diacid)[CNTATC]ATQRLANFLRHSSNN(αMePhe)GPILPPTEVGSNTY-amide;
(SEQ ID NO: 11)
K(γE-γE-C18diacid)[CNTATC]ATQRLANFLRHSSNN(αMePhe)GPILPPTEVGSNTY-amide;
(SEQ ID NO: 12)
K(γE-C18diacid)[CNTATC]ATQRLANFLRHSSNN(αMePhe)GPILPPTNVGSNTY-amide;
(SEQ ID NO: 13)
K(γE-γE-C18diacid)[CNTATC]ATQRLANFLRHSSNN(αMePhe)GPILPPTNVGSNTY-amide;
(SEQ ID NO: 14)
K(γE-γE-C18diacid)[CNTATC]ATQRLANFLVHSSNN(αMePhe)GPILPPTNVGSRTY-amide;
(SEQ ID NO: 15)
K(γE-γE-C18diacid)[CNTATC]ATQRLANFLVHSSNN(αMePhe)GPILPPTRVGSNTY-amide;
(SEQ ID NO: 16)
K(γE-C18diacid)[CNTATC]ATQRLANFLVHSSNN(αMePhe)GPILPPTRVGSNTY-amide;
(SEQ ID NO: 17)
K(γE-C18diacid)K[CNTATC]ATQRLA(Dab)FLVHSSNNFGPILPPTNVGSNTY-amide;
(SEQ ID NO: 18)
K(γE-C18diacid)K[CNTATC]ATQRLANFLVHSS(Aib)NFGPILPPTHVGSNTY-amide;
K(γE-C18diacid)[CNTATC]ATQRLANFLRHSS(Aib)NFGPILPPTEVGSNTY-amide;
(SEQ ID NO: 19)
(SEQ ID NO: 20)
K(γE-C18diacid)[CNTATC]ATQRLANFLRHSS(Aib)NFGPILPPTNVGSNTY-amide;
(SEQ ID NO: 21)
K(γE-γE-C18diacid)K[CNTATC]ATQRLANFLRHSS(Aib)NFGPILPPTNVGSNTY-amide;
(SEQ ID NO: 22)
K(γE-γE-C18diacid)K[CNTATC]ATQRLANFLVHSS(Aib)NFGPILPPTRVGSNTY-amide;
(SEQ ID NO: 23)
K(γE-C18diacid)K[CNTATC]ATQRLANFLVHSS(Aib)NFGPILPPT(Aib)VGSNTY-amide;
(SEQ ID NO: 24)
K(γE-C18diacid)K[CNTATC]ATQRLANFL(Aib)HSSNNFGPILPPTNVGSNTY-amide;
and
(SEQ ID NO: 25)
K(γE-C18diacid)K[CNTATC]ATQRLA(Dab)FL(Aib)HSSNNFGPILPPTEVGSNTY-amide.

The AMYR agonist polypeptide may comprise or consist of an amino acid sequence having at least 90%, at least 95%, at least 97%, at least 99%, or 100% identity to the amino acid sequence K[CNTATC]ATQRLANFLRHSSNN (aMePhe) GPILPPTEVGSNTY-amide (SEQ ID NO: 26).

The AMYR agonist polypeptide, or pharmaceutically acceptable salt thereof may comprise or consist of the amino acid sequence K (γE-γE-C18diacid)[CNTATC]ATQRLANFLRHSSNN (aMePhe) GPILPPTEVGSNTY-amide (SEQ ID NO: 11).

The GLP-1R agonist may be a GLP-1R agonist polypeptide, or pharmaceutically acceptable salt thereof, which comprises or consists of an amino acid sequence having at least 60%, at least 65%, at least 70%, at least 75%, at least 80%, at least 85%, at least 90%, at least 95%, or at least 97% identity to cotadutide (HSQGTFTSD-X10-SEYLDSERARDFVAWLEAGG-acid, wherein X10=Lys[ε-γE-Palmitoyl], SEQ ID NO: 32); optionally wherein the GLP-1R agonist comprises an amino acid sequence having at least 65% identity to cotadutide.

The GLP-1R agonist polypeptide, or pharmaceutically acceptable salt thereof, may comprise or consist of an amino acid sequence having at least 90%, at least 95%, at least 97%, at least 99%, or the 100% identity to amino acid sequence H(Aib)QGTFTSDVSK(αMePhe)LDTKRARDFVQWLLE(Aib)G-acid (SEQ ID NO: 33).

The GLP-1R agonist polypeptide, or pharmaceutically acceptable salt thereof, may be lipidated, optionally wherein the lipid is selected from the group consisting of C18diacid, or C20diacid, optionally wherein the lipid is octadecanedioic acid (C18diacid).

The lysine at position 17 of the GLP-1R agonist polypeptide may be lipidated and/or acylated; optionally wherein the lipid is linked to the epsilon amino group of lysine at position 17 of the GLP-1R agonist polypeptide via a linker; further optionally wherein the lysine at position 17 of the GLP-1R agonist polypeptide is acylated and lipidated, the lipid is linked to the acylated lysine via its epsilon amino group to ((O2Oc)-(O2Oc)-γE) linker in the C- to N-terminal orientation, and the lipid is octadecanedioic acid (C18diacid).

The GLP-1R agonist polypeptide, or pharmaceutically acceptable salt thereof, may comprise or consist of the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

The invention also provides a method for treating and/or preventing a disease or disorder in a subject, the method comprising administering to the subject (a) a polypeptide, or pharmaceutically acceptable salt thereof, which is an AMYR agonist comprising an amino acid sequence having at least 90% to pramlintide identity (KCNTATCATQRLANFLVHSSNNFGPILPPTNVGSNTY-amide, SEQ ID NO: 5); and (b) a GLP-1R agonist.

In said method, the AMYR agonist may be as defined herein, optionally wherein the AMYR agonist polypeptide, or pharmaceutically acceptable salt thereof, comprises or consists of the amino acid sequence K(γE-γE-C18diacid)[CNTATC]ATQRLANFLRHSSNN(αMePhe)GPILPPTEVGSNTY-amide (SEQ ID NO: 11).

In said method: (a) the GLP-1R agonist may be as defined herein; and/or (b) the method may further comprise administering to the subject a GCGR agonist.

The invention also provides a method for treating and/or preventing a disease or disorder in a subject, the method comprising administering to the subject (a) an AMYR agonist; and (b) a polypeptide, or pharmaceutically acceptable salt thereof, which is a GLP-1R agonist comprising or consisting of an amino acid sequence having at least 60%, at least 65%, at least 70%, at least 75%, at least 80%, at least 85%, at least 90%, at least 95%, or at least 97% identity to cotadutide (HSQGTFTSD-X10-SEYLDSERARDFVAWLEAGG-acid, wherein X10=Lys[ε-γE-Palmitoyl], SEQ ID NO: 32); optionally wherein the GLP-1R agonist comprises an amino acid sequence having at least 65% identity to cotadutide.

In said method, the GLP-1R agonist may be as defined herein, optionally wherein the GLP-1R agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

In said method: (a) the AMYR agonist may be as defined herein; and/or (b) the method may further comprise administering to the subject a GCGR agonist.

In a method of the invention, the AMYR agonist polypeptide, or pharmaceutically acceptable salt thereof, may comprise or consist of the amino acid sequence K (γE-γE-C18diacid)[CNTATC]ATQRLANFLRHSSNN(αMePhe)GPILPPTEVGSNTY-amide (SEQ ID NO: 11); and the GLP-1R agonist polypeptide, or pharmaceutically acceptable salt thereof, may comprise or consist of the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

A method of the invention may be used to treat or prevent obesity.

A method of the invention may be used to treat or prevent an obesity-related condition; optionally wherein the obesity-related condition is overweight, morbid obesity, obesity prior to surgery, obesity-linked inflammation, obesity-linked gallbladder disease, sleep apnoea and respiratory problems, hyperlipidaemia, degeneration of cartilage, osteoarthritis, or reproductive health complications of obesity or overweight such as infertility.

A method of the invention may be used to treat or prevent a metabolic disease, optionally wherein the metabolic disease includes diabetes, type 1 diabetes, type 2 diabetes, gestational diabetes, pre-diabetes, insulin resistance, impaired glucose tolerance (IGI), disease states associated with elevated blood glucose levels, metabolic syndrome, or hyperglycemia (e.g. abnormal postprandial hyperglycemia).

A method of the invention may be used to treat or prevent hepatic steatosis (“fatty liver”), e.g., Metabolic dysfunction associated steatohepatitis (MASH).

A method of the invention may further comprise treating, improving, and/or protecting liver and/or kidney function in the subject. The method of treating, improving, and/or protecting liver function may comprise or consist of reducing steatohepatitis and/or fibrosis in the liver.

The invention further provides a method of inhibiting or reducing weight gain, promoting weight loss, reducing food intake, increasing satiety, and/or reducing excess body weight in a subject, the method comprising administering the AMYR agonist, or pharmaceutically acceptable salts thereof, as defined herein and the GLP-1R agonist, or pharmaceutically acceptable salts thereof, as defined herein, to the subject; optionally wherein: (a) the GLP-1R agonist activates both a GLP-1R and a GCGR; optionally wherein the GLP-1R has selectivity to GLP-1R as compared to GCGR; or (b) the method further comprises administering to the subject a GCGR agonist.

The invention also provides a cosmetic method of inhibiting or reducing weight gain, promoting weight loss, reducing food intake, increasing satiety, and/or reducing excess body weight in a subject, the method comprising administering the AMYR agonist, or pharmaceutically acceptable salts thereof, as defined herein and the GLP-1R agonist, or pharmaceutically acceptable salts thereof, as defined herein, to the subject; optionally wherein: (a) the GLP-1R agonist activates both a GLP-1R and a GCGR; optionally wherein the GLP-1R has selectivity to GLP-1R as compared to GCGR; or (b) the method further comprises administering to the subject a GCGR agonist.

Thus, the invention provides a method of reducing food intake in a subject, the method comprising administering the AMYR agonist, or pharmaceutically acceptable salts thereof, as defined herein and the GLP-1R agonist, or pharmaceutically acceptable salts thereof, as defined herein, to the subject; optionally wherein: (a) the GLP-1R agonist activates both a GLP-1R and a GCGR; optionally wherein the GLP-1R has selectivity to GLP-1R as compared to GCGR; or (b) the method further comprises administering to the subject a GCGR agonist.

The invention also provides a cosmetic method of reducing food intake in a subject, the method comprising administering the AMYR agonist, or pharmaceutically acceptable salts thereof, as defined herein and the GLP-1R agonist, or pharmaceutically acceptable salts thereof, as defined herein, to the subject; optionally wherein: (a) the GLP-1R agonist activates both a GLP-1R and a GCGR; optionally wherein the GLP-1R has selectivity to GLP-1R as compared to GCGR; or (b) the method further comprises administering to the subject a GCGR agonist.

The invention further provides a method of reducing fat-mass specific body weight in a subject, the method comprising administering the AMYR agonist, or pharmaceutically acceptable salts thereof, as defined herein and the GLP-1R agonist, or pharmaceutically acceptable salts thereof, as defined herein, to the subject; optionally wherein: (a) the GLP-1R agonist activates both a GLP-1R and a GCGR; optionally wherein the GLP-1R has selectivity to GLP-1R as compared to GCGR; or (b) the method further comprises administering to the subject a GCGR agonist.

The invention also provides a cosmetic method of reducing fat-mass specific body weight in a subject, the method comprising administering the AMYR agonist, or pharmaceutically acceptable salts thereof, as defined herein and the GLP-1R agonist, or pharmaceutically acceptable salts thereof, as defined herein, to the subject; optionally wherein: (a) the GLP-1R agonist activates both a GLP-1R and a GCGR; optionally wherein the GLP-1R has selectivity to GLP-1R as compared to GCGR; or (b) the method further comprises administering to the subject a GCGR agonist.

The invention also provides a method of improving glycemic and/or metabolic control in a subject, the method comprising administering the AMYR agonist, or pharmaceutically acceptable salts thereof, as defined herein and the GLP-1R agonist, or pharmaceutically acceptable salts thereof, as defined herein, to the subject; optionally wherein: (a) the GLP-1R agonist activates both a GLP-1R and a GCGR; optionally wherein the GLP-1R has selectivity to GLP-1R as compared to GCGR1; or (b) the method further comprises administering to the subject a GCGR agonist.

The method of improving glycemic and/or metabolic control in a subject may comprise or consist of increasing insulin secretion, delaying gastric emptying, increasing mitochondria function, inhibiting de novo lipogenesis, decreasing HbAlc, enhancing fatty oxidation, decreasing hepatic mitochondrial oxidative stress, decreasing steatosis, decreasing fibrosis, decreasing glycogen synthesis, increasing gluconeogenesis, reducing or reversing fibrosis (e.g., liver fibrosis), reducing steatohepatitis, and/or reducing risk of death due to cirrhosis, hepatocellular carcinoma, and/or cardiorenal disease in the subject.

Thus, the invention provides a method of delaying gastric emptying in a subject, the method comprising administering the AMYR agonist, or pharmaceutically acceptable salts thereof, as defined herein and the GLP-1R agonist, or pharmaceutically acceptable salts thereof, as defined herein, to the subject; optionally wherein: (a) the GLP-1R agonist activates both a GLP-1R and a GCGR; optionally wherein the GLP-1R has selectivity to GLP-1R as compared to GCGR1; or (b) the method further comprises administering to the subject a GCGR agonist.

The method of improving glycemic and/or metabolic control in a subject may comprise or consist of reducing endogenous leptin levels and/or improving or restoring leptin sensitivity in the subject. Hyperleptinemia (e.g, in obese subjects) is associated with leptin insensitivity; thus, a reduction in endogenous leptin levels in a subject may be a factor in restoring leptin sensitivity in said subject.

In a method of the invention, the AMYR agonist and GLP-1R agonist, or pharmaceutically acceptable salts thereof, may be administered to the subject orally or by subcutaneous injection.

In a method of the invention, the AMYR agonist and GLP-1R agonist, or pharmaceutically acceptable salts thereof, may be administered to the subject by self-administration.

In a method of the invention, the AMYR agonist and GLP-1R agonist, or pharmaceutically acceptable salts thereof, may be administered about once every 4, 5, 6, 7, 8, 9, 10, 11, 12, 13, or 14 days; optionally wherein the AMYR agonist and GLP-1R agonist, or pharmaceutically acceptable salts thereof, may be administered about once a week.

In a method of the invention, the AMYR agonist and GLP-1R agonist, or pharmaceutically acceptable salts thereof, may be administered simultaneously.

In a method of the invention, the AMYR agonist and GLP-1R agonist, or pharmaceutically acceptable salts thereof, may be administered sequentially.

In a method of the invention, the AMYR agonist and GLP-1R agonist, or pharmaceutically acceptable salts thereof, may be formulated in a dual-chamber device.

The invention also provides an article of manufacture comprising the AMYR agonist, or pharmaceutically acceptable salts thereof, as defined herein and the GLP-1R agonist, or pharmaceutically acceptable salts thereof, as defined herein.

The invention also provides a kit comprising the AMYR agonist, or pharmaceutically acceptable salts thereof, as defined herein and the GLP-1R agonist, or pharmaceutically acceptable salts thereof, as herein, optionally further comprising instructions for use. The kit may comprise or consist of a dual-chamber device.

### BRIEF DESCRIPTION OF THE DRAWINGS

FIG. 1: A Graph showing Day 20% body weight change for DIO rats treated SC once daily with Vehicle, long-acting AMYR agonist (LAA) (10 nmol/kg), GLP-1/GCG agonist (GLP-1/GCG) (5.4 nmol/kg) or combination of both (10 and 5.4 nmol/kg respectively), with an n=8/group. Data reported as mean±standard error of the mean (SEM); data analysis performed using GraphPad Prism-statistical analysis performed using One-way ANOVA with Tukey ad hoc analysis. (*p<0.05, **p<0.01, ***0<0.001, ****p<0.0001). B Graph showing Day 26 change in lean mass for DIO rats treated with LAA, GLP-1/GCG or combination of both. C Graph showing Day 26 change in fat mass for DIO rats treated with LAA, GLP-1/GCG or combination of both.

FIG. 2: Graphs showing Day 26 body composition data for DIO rats treated with long-acting LAA, GLP-1/GCG or combination of both, with an n=8/group. A Body composition measured by nuclear magnetic resonance (NMR)-Mean % fat and lean mass reflected as percentage of overall body weight and B Day 26 body weight reflected as % of non-obese control as also shown with data from study day 26.

FIG. 3: Graphs showing percent change in body weight normalized to Vehicle. Rats were dosed once daily SC for 28 days with Vehicle, GLP-1/GCG (1.5 nmol/kg or 5 nmol/kg), LAA (7.5 nmol/kg) or a combination of GLP-1/GCG and LAA (1.5 and 7.5 nmol/kg, or 5 nmol/kg and 7.5 nmol/kg, respectively) with an n=6/group. Data reported as mean±standard error of the mean (SEM); data analysis performed using GraphPad Prism-statistical analysis performed using One-way ANOVA with Tukey ad hoc analysis (*p<0.05, **p<0.01, ****p<0.0001)

FIG. 4: A Graph showing percent change in body weight normalized to Vehicle. Rats were dosed every other day (Q2D) SC for 19 days with Vehicle, GLP1-Fc (0.1 or 1 mg/kg), Fc-LAA (0.1 or 2 mg/kg) or a combination of GLP1-Fc and Fc-LAA (0.1+0.1 mg/kg, 0.1+2 mg/kg, 1+0.1 mg/kg or 1+2 mg/kg, respectively) with an n=7/group. Data reported as mean±standard error of the mean (SEM); data analysis performed using GraphPad Prism.

FIG. 5: Rats were dosed SC for 15 days with Vehicle (n=16) or GLP-1 RA agonist (GLP-1RA) (n=32) (5 nmol/kg) once-daily (QD). Beginning on day 16, rats initially treated with Vehicle either continued with Vehicle (n=8) or were switched to LAA (7.5 nmol/kg QD, n=8) administration. Rats initially receiving GLP-1RA treatment either continued with GLP-1RA, were switched to LAA (7.5 nmol/kg QD), were switched to Vehicle, or continued with GLP-1RA with addition of LAA treatment (7.5 nmol/kg QD) for duration of study (day 30), with n=8/group. A Graph showing % Day 0 body weight change over time. B Graph showing % body weight change normalised to Vehicle over time. C Change in body weight at the end of the study. D Food intake at the end of the study. E Body composition data at the end of the study. F Fasting leptin levels at the end of the study. In an additional study, obese male rats were dosed QD SC for 25 days with vehicle, LAA 7.5 nmol/kg, GLP-1RA 5 nmol/kg, or in combination. G Graph showing body weight change (%) normalised to vehicle over time. H Body composition change (absolute fat mass (L) and absolute lean mass (R), g) over time. In a further study, obese male rats were dosed QD SC for 25 days with vehicle, LAA 7.5 nmol/kg, GLP-1RA 5 nmol/kg, or in combination. I Graph showing percent body weight change normalized vehicle. J Body composition fat mass change from baseline to day 27 (L), and then at treatment stop from day 27 until day 41 (R). N=8/group. **p<0.01, ***p<0.001, ****p<0.0001 vs. Vehicle, One-way ANOVA, Dunnett's analysis. K Body composition lean mass change from baseline to day 27 (L), and then at treatment stop from day 27 until day 41 (R). N=8/group. *p<0.05, ***p<0.001, vs. Vehicle, One-way ANOVA, Dunnett's analysis. Data reported as mean±standard error of the mean (SEM); data analysis performed using GraphPad Prism.

FIG. 6: DIO rats were dosed once daily SC for 28 days (n=8/group) with either Vehicle, LAA (10 nmol/kg), GLP/GIP dual agonist (2, 10 or 30 nmol/kg), or a combination of LAA (10 nmol/kg for all groups) and GLP/GIP dual agonist (2, 10 or 30 nmol/kg). Control rats were dosed SC with Vehicle. Graphs showing: A Body weight during study over time. B Baseline-corrected body weight change (%). C Body composition data represented as change in fat mass (g). D Body composition data represented as change in lean mass (g). E Body composition data represented as percentage fat mass (%) of total body weight at both week −1 (baseline) or week 4 (end of study). F Body composition data represented as percentage lean mass (%) of total body weight at both week-1 (baseline) or week 4 (end of study). Statistical analysis performed using two-way RM ANOVA with Dunnett analysis (***p<0.001, ****p<0.0001).

FIG. 7: A Graph showing result of saccharin conditioned tasted aversion experiment. Data reported as mean±standard error of the mean (SEM); data analysis performed using GraphPad Prism-statistical analysis performed using One-way ANOVA with Tukey ad hoc analysis. (*p<0.05, **p<0.01, ***0<0.001, ****p<0.0001 vs. Vehicle). B Emax model showing relationship between calcitonin receptor engagement (L) or amylin receptor engagement (R) (fold in vitro potency normalised for fraction unbound) with saccharine aversion in lean rats.

FIG. 8: DIO rats were dosed QD for 4 weeks with either Vehicle, LAA (2.5 or 10 nmol/kg), or DACRA (1, 3, 10 or 20 nmol/kg). A Graph showing body weight change (%) normalized to vehicle over time. B. Body composition change (fat mass (L) and fat-free mass (R), g) at end of study. C Total body weight change (%) normalized to vehicle and corresponding proportion of fat mass loss and fat-free mass-loss at end of study. D Emax model showing relationship between calcitonin receptor engagement with lean-mass change (g, corrected for control) (L) and amylin receptor engagement with fat-mass change (g, corrected for control) (R).

### DETAILED DESCRIPTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognise. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The headings provided herein are not limitations of the various aspects or embodiments of this disclosure.

As used herein, the term “capable of” when used with a verb, encompasses, or means the action of the corresponding verb. For example, “capable of activating” also means activates, “capable of agonising” also means agonises, “capable of binding” also means binds and “capable of specifically activating . . . ” also means specifically activates.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g., to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e., inactive, or non-immunogenic ingredients).

Embodiments described herein as “comprising” one or more features may also be considered as disclosure of the corresponding embodiments “consisting of” and/or “consisting essentially of” such features. of the corresponding embodiments “consisting of” such features.

Amino acids are referred to herein using the name of the amino acid, the three-letter abbreviation, or the single letter abbreviation. Unless otherwise indicated, amino acid sequences are written left to right in amino to carboxy orientation.

The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

A “fragment” of a polypeptide typically comprises at least 50%, at least 60%, at least 70%, at least 80%, at least 90%, at least 95%, at least 97% or more of the original polypeptide.

A “variant” amino acid sequence has substantial homology or substantial similarity to a reference amino acid sequence (or a fragment thereof). A amino acid sequence or fragment thereof is “substantially homologous” (or “substantially identical”) to a reference sequence if, when optimally aligned (with appropriate amino acid insertions or deletions) with the other amino acid (or its complementary strand), there is nucleotide sequence identity in at least about 70%, 75%, 80%, 85%, 90%, 91%, 92%, 93%, 94%, 95%, 96%, 97%, 98%, 99% or more % of the amino acids. Methods for homology determination of amino acid sequences are known in the art. Typically, a variant polypeptide of the invention retains the function or activity of the full-length polypeptide.

A variant polypeptide may be one in which amino acid residues from one species are substituted for the corresponding residue in another species, either at the conserved or non-conserved positions. Variants of amylin receptor agonists and/or GLP-1R agonists disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of DNMTs can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, lan H. et al Data Mining: Practical Machine Learning Tools and Techniques with Java Implementations. Morgan Kaufmann; (Oct. 11, 1999), ISBN: 1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of a DNMT can be derived from empirical and theoretical models (for example, analysis of likely contact residues or calculated physicochemical property) of the DNMT sequence, functional and three-dimensional structures and these properties can be considered individually and in combination.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a DNMT of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

As used herein, the terms “polynucleotides”, “nucleic acid” and “nucleic acid sequence” refers to any molecule, preferably a polymeric molecule, incorporating units of ribonucleic acid, deoxyribonucleic acid, or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other examples of nucleic acid molecules are RNA, including siRNA, shRNA, and antisense oligonucleotides. Typically, the methods of the invention relate to the production of oligonucleotides (short DNA or RNA sequences typically less than about 300 bases in length).

Unless otherwise indicated, any nucleic acid sequences are written left to right in 5′ to 3′ orientation.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually, the DNA constructs will be suitable for autonomous replication in a unicellular host, such as yeast or bacteria, but may also be intended for introduction to and integration within the genome of a cultured insect, mammalian, plant, or other eukaryotic cell lines.

The polynucleotides of the present invention may also be produced by chemical synthesis, e.g., by the phosphoramidite method or the tri-ester method and may be performed on commercial automated oligonucleotide synthesisers. A double-stranded fragment may be obtained from the single stranded product of chemical synthesis either by synthesising the complementary strand and annealing the strand together under appropriate conditions or by adding the complementary strand using DNA polymerase with an appropriate primer sequence.

In view of the degeneracy of the genetic code, considerable sequence variation is possible among the polynucleotides of the present invention. One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85%, 90%, 91%, 92%, 93%, 94%, 95%, 96%, 97%, 98%, 99% or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridising under stringent (e.g., highly stringent) hybridisation conditions. Nucleic acid sequence hybridisation will be affected by such conditions as salt concentration (e.g. NaCl), temperature, or organic solvents, in addition to the base composition, length of the complementary strands, and the number of nucleotide base mismatches between the hybridising nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g., at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). Typically, a fragment as defined herein retains the same function as the full-length polynucleotide.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators) and is in a form suitable for use within genetically engineered protein production systems. When applied to a protein, such as an amylin receptor agonist or GLP-1R agonist amino acid sequence, the term “isolated” denotes that the protein has been removed from its natural cellular milieu and is thus free of other extraneous or unwanted coding proteins and/or genetic material and is in a form suitable for use within genetically engineered protein production systems. Such isolated molecules are those that are separated from their natural environment. In the context of the invention, an isolated protein nucleic acid is one which has been separated from one or more of the reagents used in its production according to methods of the invention; one which has been separated from the other proteins and/or nucleic acid sequences synthesised in the same iteration of the method and/or one which has been separated from the one or more units on which it was synthesised.

The terms “decrease”, “reduce”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e., abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, at least about 95%, or at least about 98%, or at least about 99%, or at least about 100%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level.

As used herein, the term “sample” refers to a sample of biological materials (cells, tissue, fluid, etc.) obtained from an individual. The sample may be any suitable biological material, for example blood, plasma, saliva, serum, sputum, urine, cerebral spinal fluid, cells, a cellular extract, a tissue sample, a tissue biopsy, a stool sample and the like. Typically, the sample is blood sample. The precise biological sample that is taken from the individual may vary, but the sampling preferably is minimally invasive and is easily performed by conventional techniques. The sample may be a whole blood sample, a purified peripheral blood leukocyte sample or a cell type sorted leukocyte sample, such as a sample of the individual's neutrophils.

The term “pharmaceutically acceptable” as used herein means approved by a regulatory agency of the Federal or a state government, or listed in the U.S. Pharmacopeia, European Pharmacopeia, or other generally recognised pharmacopeia.

The publications discussed herein are provided solely for their disclosure prior to the filing date of the present application. Nothing herein is to be construed as an admission that such publications constitute prior art to the claims appended hereto. All documents cited herein are each entirely incorporated by reference herein, including all data, tables, figures, and text presented in the cited documents.

### Amylin Receptor Agonists

The invention relates to combination therapies comprising an amylin receptor (AMYR) agonist. An AMYR agonist is a molecule that is capable of binding to, and inducing signalling by, one or more receptors or receptor complexes regarded as physiological receptors for human amylin.

### Efficacy

Typically the AMYR is human AYMR (hAMYR). Human AMYR is formed of the human calcitonin receptor hCTR complexed with at least one of the human receptor activity modifying proteins designated hRAMP1, hRAMP2 and hRAMP3. Thus, hAMY1R is a complex of hCTR and hRAMP1; hAMY2R is a complex of hCTR and hRAMP2; and hAMY3R is a complex of hCTR and hRAMP3. An exemplary amino acid sequence for hRAMP1 is given in SEQ ID NO: 1. An exemplary amino acid sequence for hRAMP2 is given in SEQ ID NO: 2. An exemplary amino acid sequence for hRAMP3 is given in SEQ ID NO: 3. An exemplary amino acid sequence for hCTR is given in SEQ ID NO: 4.

An AMYR agonist of the invention may activate one or more of hAMY1R, hAMY2R and/or hAMY3R. Thus, an AMYR agonist of the invention may activate hAMY1R; hAMY2R; hAMY3R; hhAMY1R and AMY2R; hAMY1R and AhMY3R; hAMY2R and hAMY3R; or hAMY1R, hAMY2R and hAMY3R.

An AMYR agonist is not native amylin but exhibits activity at the AMYR of about at least 1% or more relative to native amylin. In some embodiments, an AMYR agonist exhibits activity at the AMYR of at least 5%, 10%, 20%, 30%, 40%, 50%, 60%, 70%, 80%, 90%, 95%, or more relative to native amylin. This may be measured using a CAMP assay, and quantified by Effective Concentration (EC) values, such as asymptotic maximum attainable response, Eₘₐₓ, or EC₅₀, as described further below.

The ability to induce CAMP formation as a result of binding to the relevant receptor or receptor complex is typically regarded as indicative of agonist activity. Other intracellular signaling pathways or events may also be used as readouts for amylin receptor agonist activity. These may include calcium release, arrestin recruitment, receptor internalization, kinase activation or inactivation, lipase activation, inositol phosphate release, diacylglycerol release or nuclear transcription factor translocation.

EC₅₀ values may be used as a measure of agonist potency at a given receptor. An EC₅₀ value is a measure of the concentration of a compound required to achieve half of that compound's maximal activity in a particular assay, for example a CAMP assay as described in Example 2 of WO 2022/129254, which is herein incorporated by reference in its entirety.

In particular, an AMYR agonist of the invention has selectivity to AMYR (preferably hAMYR) over a CTR (preferably hCTR). Such amylin agonists are described as selective amylin receptor agonists (SARAs). Thus, in some embodiments, the AMYR agonist of the invention is a SARA. In some embodiments, the AMYR agonist of the invention is not a dual amylin and calcitonin receptor agonist (DACRA). A SARA may have selectivity for AMYR over a CTR as described further below in the context of AMYR agonists of the invention.

An AMYR agonist of the invention may exhibit greater or similar selectivity to hAMYR over hCTR as pramlintide, optionally as measured using CAMP release from binding to hAMYR and hCTR. Pramlintide exhibits at least 10-fold selectivity to hAMYR as compared to hCTR.

An AMYR agonist of the invention may exhibit a lower or similar selectivity to hAMYR over hCTR as pramlintide, optionally as measured using cAMP release from binding to hAMYR and hCTR. Where an AMYR agonist of the invention exhibits a lower selectivity to hAMYR over hCTR compared with pramlintide, the AMYR agonist of the invention is still selective for hAMYR over hCTR. Pramlintide exhibits at least 10-fold selectivity to hAMYR as compared to hCTR.

An AMYR agonist of the invention may have at least a 2-fold, at least 3-fold, at least 4-fold, at least 5-fold, at least 6-fold, at least 7-fold, at least 8-fold, at least 9-fold, at least 10-fold, at least 12-fold, at least 15-fold, at least 17-fold, at least 20-fold, or at least 25-fold, at least about 50-fold, at least about 75-fold, or at least about 100-fold selectivity to hAMYR over hCTR. In preferred embodiments, the AMYR agonist has at least a 5-fold or at least a 10-fold selectivity to AMYR (e.g. hAMYR) over CTR (e.g. hCTR).

In some embodiments, an AMYR agonist of the invention has around 12-20-fold, around 14-18-fold, optionally around 16-fold selectivity to AMYR (e.g., hAMYR) over CTR (e.g. hCTR).

In some embodiments, the AMYR agonist of the invention has an EC₅₀ measured under the conditions described in Example 2 of WO 2022/129254 (i.e. containing 0.1% bovine serum albumin (BSA)) of below about 1.4 nM, below about 1.2 nM, below about 1 nM, below about 0.8 nM, below about 0.6 nM, below about 0.4 nM, below about 0.3 nM, or below about 0.2 nM for an AMYR, particularly AMY3R.

In contrast, a DACRA may have less than 2-fold selectivity, less than 1.5-fold selectivity, or less than 1.2-fold selectivity for hAMYR over hCTR. A DACRA may have approximately equal selectivity for hAMYR and hCTR. A DACRA may have at least a 1.2-fold, at least a 1.5-fold, or at least a 2-fold selectivity to hCTR over hAMYR.

### Classification

The AMYR agonist of the invention may be a polypeptide, a small molecule drug, an antibody, an antibody-drug conjugate (ADC), or an aptamer; or a pharmaceutically acceptable salt thereof.

The AMYR agonist of the invention may be a small molecule drug. As used herein a “small molecule drug” refers to a low molecular weight compound, typically an organic compound. Typically, a small molecule has a maximum molecule weight of 900 Da, allowing for rapid diffusion across cell membranes. In some embodiments, the maximum molecular weight of a small molecule is 500 Da. Typically a small molecule has a size in the order of 1 nm.

The AMYR agonist of the invention may be an aptamer. Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers. In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

The AMYR agonist of the invention may be an antibody. As used herein, the term antibody encompasses the use of a monoclonal antibody or polyclonal antibody, as well as the antigen-binding fragments of a monoclonal or polyclonal antibody, or a peptide which binds to REV-ERB with specificity. The antibody may be a Fab, F(ab′)2, Fv, scFv, Fd, Fc or dAb.

The AMYR agonist of the invention may be an ADC, in which a pharmaceutically active moiety is conjugated, directly or indirectly (e.g. via a linker) to an antibody or fragment thereof. For example, an AMYR agonist may be an amylin analogue conjugated, directly or indirectly, to an Fc fragment. The linker may be cleavable or non-cleavable.

Preferably, an AMYR agonist of the invention is a polypeptide. An AMYR agonist polypeptide may be of between about 15 to about 40 amino acids in length, preferably between about 30 to about 40 amino acids in length, most preferably about 37 amino acids in length. For the avoidance of doubt, any and all disclosure herein in relation to AMYR agonists of the invention applies particularly to AMYR agonist polypeptides unless expressly stated to the contrary.

### AMYR Agonist Polypeptides

Throughout this specification, amino acid positions of an AMYR agonist polypeptide (e.g. lipidated AMYR agonist polypeptides) are numbered according to the corresponding position in pramlintide having the sequence set forth in SEQ ID NO: 5.

Throughout this specification, amino acids are referred to by their conventional three-letter or single-letter abbreviations (e.g. Ala or A for alanine, Arg or R for arginine, etc.). In the case of certain less common or non-naturally occurring amino acids (i.e. amino acids other than the 20 encoded by the standard mammalian genetic code), unless they are referred to by their full name, frequently employed three- or four-character codes are employed for residues thereof, including αMeSer ((S)-2-amino-3-hydroxy-2-methylphenylpropanoic acid), αMePhe ((S)-2-amino-2-methyl-3-phenylpropanoic acid), Aib (2-amino-2-methylpropanoic acid), Dab (2,4-diaminobutanoic acid) and γ-Glu (γ-glutamic acid, γE).

An AMYR agonist polypeptide of the invention may comprise or consist of an amino acid sequence having at least 70%, at least 80%, at least 90%, at least 95%, at least 96%, at least 97%, at least 98%, at least 99% or more, up to 100% identity to pramlintide (KCNTATCATQRLANFLVHSSNNFGPILPPTNVGSNTY-amide, SEQ ID NO: 5). Preferably, an AMYR agonist polypeptide of the invention may comprise or consist of an amino acid sequence having at least 90%, at least 95%, at least 96%, at least 97%, at least 98%, at least 99% or more, up to 100% identity to pramlintide (KCNTATCATQRLANFLVHSSNNFGPILPPTNVGSNTY-amide, SEQ ID NO: 5).

AMYR agonist polypeptides disclosed here can be formulated in or chemically conjugated to e.g. a protein, polymeric drug carrier or advance drug delivery system that further enhance the chemical stability and or physical stability and or the circulatory exposure of the AMYR agonist polypeptide.

In embodiments of any aspect of the invention, the polypeptides (e.g. lipidated polypeptides) of the invention are isolated polypeptides (e.g. isolated lipidated polypeptides).

### Modified Amino Acids

An AMYR agonist polypeptide (e.g. lipidated polypeptides) of the invention may comprise one or more amino acid modifications or substitutions compared to the pramlintide sequence (SEQ ID NO: 5). The AMYR agonist polypeptide may comprise one or more non-proteinogenic amino acids, particularly 2,4-diaminobutanoic acid (Dab). Alternatively or in addition, the AMYR agonist polypeptide comprises one or more alpha methyl amino acids, particularly selected from 2-amino-2-methylpropanoic acid (Aib) and/or alpha methyl phenylalanine(αMePhe or aMeF). The reference to αMePhe and aMeF herein refers to(S)-2-amino-2-methyl-3-phenylpropanoic acid.

### Lipidation and Linkers

An AMYR agonist polypeptide of the invention may be lipidated. Preferably, lipidated polypeptides may have extended half-life compared to pramlintide but without the fibril-forming tendency of lipidated pramlintide analogues described in the art. Without being bound by theory, it is thought that the lipid acts as an albumin binding moiety and protects the AMYR agonist polypeptide against clearance and degradation, thereby extending the half-life of the AMYR agonist polypeptide.

Accordingly, in some embodiments, the lipid may comprise a hydrocarbon chain having from 10 to 26 C atoms, e.g. from 14 to 24 C atoms, e.g. from 16 to 22 C atoms. For example, the hydrocarbon chain may contain 14, 15, 16, 17, 18, 19, 20, 21, 22, 23 or 24 C atoms. In preferred embodiments, the lipid has 18 to 20 C atoms. In particular, the lipid may have 18 C atoms or 20 C atoms. The hydrocarbon chain may be linear or branched, and may be saturated or unsaturated. Furthermore, it can include a functional group at the end of the lipophilic chain, e.g. a carboxylic acid group which may or may not be protected during synthesis.

In some preferred embodiments, the AMYR agonist polypeptide of the invention is lipidated with a lipid is selected from the group consisting of C12diacid, C14diacid, C16diacid, C17diacid, C18diacid, C19diacid or C20diacid. In some particularly preferred embodiments, the lipid is C18diacid or C20diacid.

The AMYR agonist polypeptide may comprise at least one lipidated amino acid residue. In some embodiments, the AMYR agonist polypeptide comprises at least two lipidated amino acid residues. In preferred embodiments, the AMYR agonist polypeptide contains only one lipidated amino acid residue. The lipid may be attached to an amino acid residue of the polypeptide.

In some embodiments, the lipid is attached to the amino acid residue through a linker (referred to herein as “linker-lipid”). In alternative embodiments, the lipid is directly attached to the amino acid residue without an intervening linker. The lipid may be attached to the amino acid residue via an ester, a sulfonyl ester, a thioester, an amide, an amine or a sulphonamide. Accordingly, it will be understood that the lipid or the linker includes an acyl group, a sulphonyl group, an N atom, an O atom or an S atom which forms part of the ester, sulphonyl ester, thioester, amide, amine or sulphonamide. Optionally, an acyl group in the lipid or linker forms part of an amide or ester with the amino acid residue. Accordingly, in preferred embodiments the lipid is attached to an acylation site on the amino acid residue.

The linker may be attached to any residue of the AMYR agonist polypeptide. In some embodiments, the linker is attached to the side chain of an amino acid residue in the polypeptide, for example to the ε-N of a lysine residue. Preferably, the linker is attached to the N-terminus of the polypeptide, (e.g. to a lysine at the N-terminus of the polypeptide). The linker may comprise one or more residues of any naturally occurring or non-naturally occurring amino acid. The linker may comprise a combination of residues, as single or repeating units. For example, the linker may comprise multiple combinations of residues, as single or repeating units, each of which may independently be a residue of Glu (E), y-Glu (γE), Lys, ε-Lys, Asp, β-Asp, Gaba, β-Ala β-aminopropanoyl), O2Oc (2-(2-(2-aminoethoxy)ethoxy)acetic acid), PEG2 (3-(2-(2-aminoethoxy)ethoxy)propanoic acid), PEG4 (1-amino-3, 6, 9, 12-tetraoxapentadecan-15-oic acid), PEGS (1-amino-3,6,9, 12, 15, 18,21,24-octaoxaheptacosan-27-oic acid, PEG12 12, (1-amino-3,6,9, 15, 18,21,24,27,30,33,36-dodecaoxanonatriacontan-39-oic acid). y-Glu and β-Asp refer to amino acids where the alpha-amino group and the side chain carboxyl group participate in peptide bond formation. ε-Lys refers to an amino acid where the epsilon-amino and carboxyl group of lysine participate in peptide bond formation.

In some embodiments, an AMYR agonist polypeptide comprises a lipid is attached to an amino acid residue in the AMYR agonist polypeptide by a linker, wherein the linker comprises y-glutamic acid (γE), γE-γE, ((O2Oc)-(O2Oc)-γE), ((O2Oc)-(O2Oc)-γEγE), or ((PEG2)-(PEG2)-γE). In some embodiments, an AMYR agonist polypeptide comprises a lipid is attached to an amino acid residue in the AMYR agonist polypeptide by a linker, wherein the linker consists of γE, γE-γE, ((O2Oc)-(O2Oc)-γE), ((O2Oc)-(O2Oc)-γEγE), or ((PEG2)-(PEG2)-γE).

Combinations of linker, lipid and polypeptide acylation sites are described in WO 2022/129254.

In some preferred embodiments, the lysine at position 1 of the AMYR agonist polypeptide, or pharmaceutically acceptable salt thereof, is lipidated. In some particularly preferred embodiments, wherein the lysine at position 1 of the AMYR agonist polypeptide is lipidated, the lipid is linked to the lysine via a γE-γE linker, and the lipid is octadecanedioic acid (C18diacid).

### Exemplary AMYR Agonist Polypeptides

In some preferred embodiments, the lipidated polypeptides of WO 2022/129254 may be used in the combination therapies of the present invention. Thus, an AMYR agonist polypeptide, or pharmaceutically acceptable salt thereof, may comprise or consist of an amino acid sequence having at least 90%, at least 95 at least 97%, at least 98%, at least 99% or more, up to 100% identity to an amino acid sequence selected from the group consisting of:

(SEQ ID NO: 6)
C18diacid-γE-[CNTATC]ATQRLAEFLRHSSNN(αMePhe)GPILPPTNVGSNTY-amide;
(SEQ ID NO: 7)
K(γE-γE-C18diacid)[CNTATC]ATQRLAEFLRHSSNN(αMePhe)GPILPPTNVGSNTY-amide;
(SEQ ID NO: 8)
K(γE-C18diacid)K[CNTATC]ATQRLAEFLRHSSNN(αMePhe)GPILPPTNVGSNTY-amide;
(SEQ ID NO: 9)
K(γE-C18diacid)K[CNTATC]ATQRLANFLRHSSNN(αMePhe)GPILPPTEVGSNTY-amide;
(SEQ ID NO: 10)
K(O2Oc-O2Oc-γE-18diacid)[CNTATC]ATQRLANFLRHSSNN(αMePhe)GPILPPTEVGSNTY-amide;
(SEQ ID NO: 11)
K(γE-γE-C18diacid)[CNTATC]ATQRLANFLRHSSNN(αMePhe)GPILPPTEVGSNTY-amide;
(SEQ ID NO: 12)
K(γE-C18diacid)[CNTATC]ATQRLANFLRHSSNN(αMePhe)GPILPPTNVGSNTY-amide;
(SEQ ID NO: 13)
K(γE-γE-C18diacid)[CNTATC]ATQRLANFLRHSSNN(αMePhe)GPILPPTNVGSNTY-amide;
(SEQ ID NO: 14)
K(γE-γE-C18diacid)[CNTATC]ATQRLANFLVHSSNN(αMePhe)GPILPPTNVGSRTY-amide;
(SEQ ID NO: 15)
K(γE-γE-C18diacid)[CNTATC]ATQRLANFLVHSSNN(αMePhe)GPILPPTRVGSNTY-amide;
(SEQ ID NO: 16)
K(γE-C18diacid)[CNTATC]ATQRLANFLVHSSNN(αMePhe)GPILPPTRVGSNTY-amide;
(SEQ ID NO: 17)
K(γE-C18diacid)K[CNTATC]ATQRLA(Dab)FLVHSSNNFGPILPPTNVGSNTY-amide;
(SEQ ID NO: 18)
K(γE-C18diacid)K[CNTATC]ATQRLANFLVHSS(Aib)NFGPILPPTHVGSNTY-amide;
(SEQ ID NO: 19)
K(γE-C18diacid)[CNTATC]ATQRLANFLRHSS(Aib)NFGPILPPTEVGSNTY-amide;
(SEQ ID NO: 20)
K(γE-C18diacid)[CNTATC]ATQRLANFLRHSS(Aib)NFGPILPPTNVGSNTY-amide;
(SEQ ID NO: 21)
K(γE-γE-C18diacid)K[CNTATC]ATQRLANFLRHSS(Aib)NFGPILPPTNVGSNTY-amide;
(SEQ ID NO: 22)
K(γE-γE-C18diacid)K[CNTATC]ATQRLANFLVHSS(Aib)NFGPILPPTRVGSNTY-amide;
(SEQ ID NO: 23)
K(γE-C18diacid)K[CNTATC]ATQRLANFLVHSS(Aib)NFGPILPPT(Aib)VGSNTY-amide;
(SEQ ID NO: 24)
K(γE-C18diacid)K[CNTATC]ATQRLANFL(Aib)HSSNNFGPILPPTNVGSNTY-amide;
and
(SEQ ID NO: 25)
K(γE-C18diacid)K[CNTATC]ATQRLA(Dab)FL(Aib)HSSNNFGPILPPTEVGSNTY-amide.

In some preferred embodiments, an AMYR agonist polypeptide comprises an amino acid sequence having at least 70%, at least 80%, at least 90%, at least 95%, at least 96%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26).

In some preferred embodiments, an AMYR agonist polypeptide consists of an amino acid sequence having at least 70%, at least 80%, at least 90%, at least 95%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26).

In some particularly preferred embodiments, an AMYR agonist polypeptide comprises an amino acid sequence having at least 90%, at least 95%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26).

In some particularly preferred embodiments, an AMYR agonist polypeptide consists of an amino acid sequence having at least 90%, at least 95%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26).

More preferably, the AMYR agonist polypeptide, or pharmaceutically acceptable salt thereof, comprises the amino acid sequence K (γE-γE-C18diacid)[CNTATC]ATQRLANFLRHSSNN(αMePhe)GPILPPTEVGSNTY-amide (SEQ ID NO: 11).

Most preferably, the AMYR agonist polypeptide, or pharmaceutically acceptable salt thereof, consists of the amino acid sequence K (γE-γE-C18diacid)[CNTATC]ATQRLANFLRHSSNN(αMePhe)GPILPPTEVGSNTY-amide (SEQ ID NO: 11).

### Pharmacokinetics

The AMYR agonist polypeptides used in the invention may exhibit favourable pharmacokinetic properties as compared to pramlintide. For example, the AMYR agonist polypeptides may have an extended half-life as compared to pramlintide.

As used herein, the term “half-life” is used to refer to the time taken for the concentration of isolated polypeptide in plasma to decline to 50% of its original level. Methods to determine the half-life of proteins are known in the art and are described in Example 4 of WO 2022/129254. It will be recognised that an extended half-life is advantageous, as it permits the therapeutic proteins to be administered according to a safe and convenient dosing schedule, e.g. lower doses that can be administered less frequently. Moreover, the achievement of lower doses may provide further advantages such as the provision of an improved safety profile. To the contrary, pramlintide requires frequent and inconvenient administration.

The AMYR agonist polypeptides may have a half-life of at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours or at least 14 hours in rat models. In preferred embodiments, the AMYR agonist polypeptides have a half-life of at least 14 hours.

### Reduced Fibrillation

The AMYR agonist polypeptides may exhibit reduced tendency to undergo fibrillation in pharmaceutically relevant aqueous media, especially at pH values in the range from 4 to 7, as compared to lipidated pramlintide. In some embodiments, the AMYR agonist polypeptides exhibit reduced tendency to undergo fibrillation in pharmaceutically relevant aqueous media, especially at pH values in the range from 4 to 7, as compared to pramlintide which is lipidated in a similar manner e.g. the same lipid is attached, the lipid is attached through the same linker and/or the lipid is attached at the same position. Exemplary lipidated AMYR agonist polypeptides, for example SEQ ID NOs: 6-25.

Accordingly, the AMYR agonist polypeptides may be suited for formulation in acidic media (e.g. pH 4) and in neutral or near-neutral media (e.g. pH 7 or 7.4). Such AMYR agonist polypeptides may be well suited for coformulation with, for example, insulin, various insulin analogues and/or other therapeutic (e.g. anti-diabetic or anti-obesity) agents that require a neutral or near-neutral formulation pH.

In some embodiments, the AMYR agonist polypeptides show no detectable fibrillation after about 5 hours, after about 7 hours, after about 9 hours, after about 11 hours, after about 13 hours, after about 15 hours, after about 17 hours or after about 20 hours, after about 48 hours, after about 72 hours, after about 96 hours, after about 108 hours, after about 120 hours, after 132 about hours or after about 144 hours at pH 4 and 37° C. Suitable assays for quantifying fibrillation are described in Example 3 of WO 2022/129254 (which describes a Thioflavin T fibrillation assay).

In preferred embodiments, the AMYR agonist polypeptides are soluble at concentrations required for therapeutic efficacy.

### GLP-1 Receptor Agonists

The invention relates to combination therapies comprising a GLP-1 receptor (GLP-1R) agonist. An GLP-1R agonist is a molecule that is capable of binding to, and inducing signalling by, one or more receptors or receptor complexes regarded as physiological receptors for human GLP-1R.

### Efficacy

Native GLP-1 refers to naturally-occurring GLP-1, typically human GLP-1, and is a generic term that encompasses, e.g., GLP-1 (7-36) amide (SEQ ID NO: 27), GLP-1 (7-37) acid (SEQ ID NO: 28), or a mixture of those two compounds. Typically the GLP-1 receptor (GLP-1R) is human GLP-1R (hGLP-1R). An exemplary hGLP-1R sequence is given in SEQ ID NO: 29.

As used herein, a general reference “GLP-1” in the absence of any further designation is intended to mean native human GLP-1. Unless otherwise indicated, “GLP-1” refers to human GLP-1. Similarly, a general reference “GLP-1R” in the absence of any further designation is intended to mean native human GLP-1R. Unless otherwise indicated, “GLP-1R” refers to human GLP-1R.

A GLP-1 receptor agonist of the invention activates GLP-1R. As used herein, a GLP-1R agonist is not native GLP-1 but exhibits activity at the GLP-1R of about at least 1% or more relative to native GLP-1. In some aspects, a GLP-1R agonist exhibits activity at the GLP-1 receptor of at least 5%, 10%, 20%, 30%, 40%, 50%, 60%, 70%, 80%, 90%, 95%, or more relative to native GLP-1. This may be measured using a CAMP assay, as described herein.

EC₅₀ values may be used as a measure of agonist potency at a given receptor, such as GLP-1R agonist potency. An EC₅₀ value is a measure of the concentration of a compound required to achieve half of that compound's maximal activity in a particular assay, for example a CAMP assay as described in Example 2 of WO 2023/148366, which is herein incorporated by reference in its entirety.

### GLP-1/GCG Dual-Agonists

In some preferred embodiments, a GLP-1R agonist of the invention may also be a glucagon receptor (GCGR) agonist. For the avoidance of doubt, all references herein to GLP-1R agonists of the invention apply equally and without reservation to GLP-1R/GCGR dual-agonists.

As used herein the term “native glucagon” refers to naturally-occurring glucagon, e.g., human glucagon, comprising the sequence of SEQ ID NO: 30. Typically the GCG receptor (GCGR) is human GCGR (hGCGR). An exemplary hGCGR sequence is given in SEQ ID NO: 31.

As used herein, a general reference to “glucagon” in the absence of any further designation is intended to mean native human glucagon 1. Unless otherwise indicated, “glucagon” refers to human glucagon. Similarly, a general reference “GCGR” in the absence of any further designation is intended to mean native human GCGR. Unless otherwise indicated, “GCGR” refers to human GCGR.

A GCGR agonist is not native GCG but exhibits activity at the GCGR of about at least 1% or more relative to native GCG. In some aspects, a GCGR agonist exhibits activity at the GCGR of at least 5%, 10%, 20%, 30%, 40%, 50%, 60%, 70%, 80%, 90%, 95%, or more relative to native GCG. Again, this may be measured using a CAMP assay, as described further herein.

An agonist with both GLP-1R and GCGR agonist activity may be referred to interchangeably as a “GLP-1R/GCGR agonist,” “GLP-1R/GCGR co-agonist,” “GLP-1R and GCGR dual agonist” or “GLP-1R and GCGR dual co-agonist”. Thus, a “GLP-1R/GCGR agonist,” “GLP-1R/GCGR co-agonist,” “GLP-1R and GCGR dual agonist” or “GLP-1R and GCGR dual co-agonist” is not native GLP-1 and is not native GCG, and exhibits activity at the GCGR of at least 1% or more relative to native GCG and also exhibits activity at the GLP-1 receptor of about at least 1% or more relative to native GLP-1. In some aspects, a “GLP-1R/GCGR agonist” or a “GLP-1 and GCG dual agonist” exhibits activity at the GCGR of at least 5%, 10%, 20%, 30%, 40%, 50%, 60%, 70%, 80%, 90%, 95%, or more relative to native GCG and also exhibits activity at the GLP-1R of about at least 5%, 10%, 20%, 30%, 40%, 50%, 60%, 70%, 80%, 90%, 95%, or more relative to native GLP-1. This may be measured using a CAMP assay, as described herein.

In some embodiments, the GLP-1R/GCGR dual-agonists the invention exhibit in vitro potencies at the GLP-1R as shown by an EC₅₀ measured under the conditions described in Example 2 of WO 2023/148366 of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

In some embodiments, the GLP-1R/GCGR dual-agonists the invention exhibit in vitro potencies at the GCGR as shown by an EC₅₀ measured under the conditions described in Example 2 of WO 2023/148366 of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

In embodiments wherein a GLP-1R agonist activates both GLP-1R and GCGR, i.e, wherein the GLP-1R agonist is a GLP-1R/GCGR dual-agonist, said GLP-1R/GCGR dual-agonist may activate GLP-1R and GCGR equally.

Preferably, in embodiments wherein a GLP-1R agonist agonises activates both GLP-1R and GCGR, i.e, wherein the GLP-1R agonist is a GLP-1R/GCGR dual-agonist, said GLP-1R/GCGR dual-agonist may have selectivity for GLP-1R as compared to GCGR.

A GLP-1R/GCGR dual-agonist of the invention may have has at least a 1.5-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, at least 17-fold, at least 20-fold, or at least 25-fold selectivity to GLP-1R as compared to GCGR. In preferred embodiments, the GLP-1R/GCGR dual-agonist has at least a 1.5-fold selectivity to GCGR as compared to GLP-1R.

The selectivity ratio of a GLP-1R/GCGR dual-agonist of the invention to GLP-1R vs GCGR is defined as: Relative Potency Ratio (RPR)=% GLP-1R activity relative to GLP-1/% GCGR activity relative to glucagon. In other words, the higher the RPR, the greater the selectivity for GLP-1R.

A GLP-1R/GCGR dual-agonist of the invention may have a hGLP-1R/hGCGR relative potency ratio (RPR) of about 1 to about 25 (with an RPR of 1 meaning the GLP-1R/GCGR dual-agonist activates GLP-1R and GCGR equally, and an RPR of 25 meaning that a GLP-1R/GCGR dual-agonist has 25-fold selectivity for GLP-1R compared with GCGR). In certain aspects, a GLP-1R/GCGR dual-agonist may have a hGLP-1R/hGCGR relative potency ratio of about 1 to about 20. In certain aspects, a GLP-1R/GCGR dual-agonist may have a hGLP1R/hGCGR relative potency ratio of about 1 to about 15. In certain aspects, a GLP-1R/GCGR dual-agonist may have a hGLP-1R/hGCGR relative potency ratio of about 1 to about 10.

A GLP-1R/GCGR dual-agonist may have a hGLP-1R/hGCGR relative potency ratio of about 2 to about 25. In certain aspects, a GLP-1R/GCGR dual-agonist may have a hGLP-1R/hGCGR relative potency ratio of about 2 to about 20. In certain aspects, a GLP-1R/GCGR dual-agonist may have a hGLP1R/hGCGR relative potency ratio of about 2 to about 15. In certain aspects, a GLP-1R/GCGR dual-agonist may have a hGLP-1R/hGCGR relative potency ratio of about 2 to about 10.

### Classification

A GLP-1R agonist of the invention (e.g. a GLP-1R/GCGR dual-agonist) may be a polypeptide, a small molecule drug, an antibody, an antibody-drug conjugate (ADC), or an aptamer; or a pharmaceutically acceptable salt thereof.

A GLP-1R agonist of the invention (e.g. a GLP-1R/GCGR dual-agonist) may be a small molecule drug. As used herein a “small molecule drug” refers to a low molecular weight compound, typically an organic compound. Typically, a small molecule has a maximum molecule weight of 900 Da, allowing for rapid diffusion across cell membranes. In some embodiments, the maximum molecular weight of a small molecule is 500 Da. Typically a small molecule has a size in the order of 1 nm.

A GLP-1R agonist of the invention (e.g. a GLP-1R/GCGR dual-agonist) may be an aptamer. Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

A GLP-1R agonist of the invention (e.g. a GLP-1R/GCGR dual-agonist) may be an antibody. As used herein, the term antibody encompasses the use of a monoclonal antibody or polyclonal antibody, as well as the antigen-binding fragments of a monoclonal or polyclonal antibody, or a peptide which binds to REV-ERB with specificity. The antibody may be a Fab, F(ab′)2, Fv, scFv, Fd, Fc or dAb.

A GLP-1R agonist of the invention (e.g. a GLP-1R/GCGR dual-agonist) may be an ADC, in which a pharmaceutically active moiety is conjugated, directly or indirectly (e.g. via a linker) to an antibody. For example, a GLP-1R agonist may be an GLP-1 analogue conjugated, directly or indirectly, to an Fc fragment. The linker may be cleavable or non-cleavable.

Preferably, a GLP-1R agonist of the invention (e.g. a GLP-1R/GCGR dual-agonist) is a polypeptide. A GLP-1R agonist polypeptide of the invention (e.g. a GLP-1R/GCGR dual-agonist polypeptide) may be of between about 15 to about 40 amino acids in length, preferably between about 25 to about 35 amino acids in length, most preferably about 30 or 31 amino acids in length. For the avoidance of doubt, any and all disclosure herein in relation to GLP-1R agonists of the invention (e.g. GLP-1R/GCGR dual-agonists) of the invention applies particularly to GLP-1R agonist polypeptides (e.g. GLP-1R/GCGR dual-agonist polypeptides) unless expressly stated to the contrary.

### GLP-1R Agonist Polypeptides

Throughout this specification, amino acid positions of a GLP-1R agonist polypeptide of the invention (e.g. a GLP-1R/GCGR dual-agonist polypeptide) are numbered according to the corresponding position in cotadutide having the sequence set forth in SEQ ID NO: 32.

A GLP-1R agonist polypeptide of the invention (e.g. a GLP-1R/GCGR dual-agonist polypeptide) may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to cotadutide (HSQGTFTSD-X10-SEYLDSERARDFVAWLEAGG-acid, wherein X10=Lys[ε-γE-Palmitoyl], SEQ ID NO: 32). In some preferred embodiments, a GLP-1R agonist polypeptide of the invention (e.g. a GLP-1R/GCGR dual-agonist polypeptide) may comprise or consist of an amino acid sequence having at least 65% identity to cotadutide (SEQ ID NO: 32).

A GLP-1R agonist polypeptide of the invention (e.g. a GLP-1R/GCGR dual-agonist polypeptide) may comprise an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33) or H(Aib)HGS(αMePhe)TSDVSK(αMePhe)LDSRAAKD(αMePhe)VQ(Aib)IANT-amide (SEQ ID NO: 34).

A GLP-1R agonist polypeptide of the invention (e.g. a GLP-1R/GCGR dual-agonist polypeptide) may consist of an amino acid sequence having at least 60%, at least 65%, at least 70%, at least 75%, at least 80%, at least 85%, at least 90%, at least 95%, at least 97%, at least 98%, at least 99% or more, up to identity 100% to H(Aib)QGTFTSDVSK(αMePhe)LDTKRARDFVQWLLE(Aib)G-acid (SEQ ID NO: 33) or H(Aib)HGS(αMePhe)TSDVSK(αMePhe)LDSRAAKD(αMePhe)VQ(Aib)IANT-amide (SEQ ID NO: 34).

A GLP-1R agonist polypeptide of the invention (e.g. a GLP-1R/GCGR dual-agonist polypeptide) may comprise an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33) or H(Aib)HGS(αMePhe)TSDVSK(αMePhe)LDSRAAKD(αMePhe)VQ(Aib)IANT-amide (SEQ ID NO: 34).

A GLP-1R agonist polypeptide of the invention (e.g. a GLP-1R/GCGR dual-agonist polypeptide) may consist of an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33) or H(Aib)HGS(αMePhe)TSDVSK(αMePhe)LDSRAAKD(αMePhe)VQ(Aib)IANT-amide (SEQ ID NO: 34).

A GLP-1R agonist polypeptide of the invention (e.g. a GLP-1R/GCGR dual-agonist polypeptide) may comprise an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

A GLP-1R agonist polypeptide of the invention (e.g. a GLP-1R/GCGR dual-agonist polypeptide) may consist of an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

A GLP-1R agonist polypeptide of the invention (e.g. a GLP-1R/GCGR dual-agonist polypeptide) may comprise an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

A GLP-1R agonist polypeptide of the invention (e.g. a GLP-1R/GCGR dual-agonist polypeptide) may consist of an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

GLP-1R agonist polypeptides of the invention (e.g. GLP-1R/GCGR dual-agonist polypeptides) disclosed here can be formulated in or chemically conjugated to e.g. a protein, polymeric drug carrier or advance drug delivery system that further enhance the chemical stability and or physical stability and or the circulatory exposure of the GLP-1R agonist polypeptides of the invention (e.g. GLP-1R/GCGR dual-agonist polypeptides).

In embodiments of any aspect of the invention, the GLP-1R agonist polypeptides of the invention (e.g. GLP-1R/GCGR dual-agonist polypeptides) of the invention are isolated polypeptides.

### Modified Amino Acids

A GLP-1R agonist polypeptide of the invention (e.g. a GLP-1R/GCGR dual-agonist polypeptide) may comprise one or more amino acid modifications or substitutions compared to the cotadutide sequence (SEQ ID NO: 32). In particular, a GLP-1R agonist polypeptide of the invention (e.g. a GLP-1R/GCGR dual-agonist polypeptide) may comprise one or more alpha methyl amino acids, particularly selected from 2-amino-2-methylpropanoic acid (Aib) and/or alpha methyl phenylalanine (aMePhe or aMeF).

### Lipidation and Linkers

A GLP-1R agonist polypeptide of the invention (e.g. a GLP-1R/GCGR dual-agonist polypeptide) may be lipidated. Without being bound by theory, it is thought that the lipid acts as an albumin binding moiety and protects the GLP-1R agonist polypeptide of the invention (e.g. a GLP-1R/GCGR dual-agonist polypeptide) against clearance and degradation, thereby extending the half-life of the GLP-1R agonist polypeptide of the invention (e.g. a GLP-1R/GCGR dual-agonist polypeptide).

Accordingly, in some embodiments, the lipid may comprise a hydrocarbon chain having from 10 to 26 C atoms. In preferred embodiments, the lipid has 18 to 20 C atoms. In particular, the lipid may have 18 C atoms or 20 C atoms. The hydrocarbon chain may be linear or branched, and may be saturated or unsaturated. Furthermore, it can include a functional group at the end of the lipophilic chain, e.g. a carboxylic acid group which may or may not be protected during synthesis.

The GLP-1R agonist polypeptide of the invention (e.g. a GLP-1R/GCGR dual-agonist polypeptide), or pharmaceutically acceptable salt thereof, may be lipidated with a lipid selected from the group consisting of C18diacid, or C20diacid. The lipid may be octadecanedioic acid (C18diacid). The lipid may be icosanedioic acid (C20diacid).

The GLP-1R agonist polypeptide of the invention (e.g. a GLP-1R/GCGR dual-agonist polypeptide) may comprise at least one lipidated amino acid residue. In some embodiments, the GLP-1R agonist polypeptide of the invention (e.g. a GLP-1R/GCGR dual-agonist polypeptide) comprises at least two lipidated amino acid residues. In preferred embodiments, the GLP-1R agonist polypeptide of the invention (e.g. a GLP-1R/GCGR dual-agonist polypeptide) contains only one lipidated amino acid residue. The lipid may be attached to an amino acid residue of the polypeptide.

In some embodiments, the lipid is attached to the amino acid residue through a linker (referred to herein as “linker-lipid”). In alternative embodiments, the lipid is directly attached to the amino acid residue without an intervening linker. The lipid may be attached to the amino acid residue via an ester, a sulfonyl ester, a thioester, an amide, an amine or a sulphonamide. Accordingly, it will be understood that the lipid or the linker includes an acyl group, a sulphonyl group, an N atom, an O atom or an S atom which forms part of the ester, sulphonyl ester, thioester, amide, amine or sulphonamide. Optionally, an acyl group in the lipid or linker forms part of an amide or ester with the amino acid residue. Accordingly, in preferred embodiments the lipid is attached to an acylation site on the amino acid residue.

In some aspects, one or more lysine residues of the GLP-1R agonist polypeptide of the invention (e.g. a GLP-1R/GCGR dual-agonist polypeptide) are acylated. In some aspects, the lysine at position 17 is acylated. In some aspects, the lysine at position 20 is acylated.

In some aspects, one or more lysine resides of the GLP-1R agonist polypeptide of the invention (e.g. a GLP-1R/GCGR dual-agonist polypeptide) are lipidated. In some aspects, the lysine at position 17 is lipidated. In some aspects, the lysine at position 20 is lipidated. In some aspects, the linker is linked to the epsilon amino group of the residue at position 17 or 20.

The linker may comprise one or more residues of any naturally occurring or non-naturally occurring amino acid, such as those described herein in relation to the AMYR agonist polypeptides. The disclosure of linkers in the context of AMYR agonist polypeptides applies equally and without reservation to linkers suitable for use with a GLP-1R agonist polypeptide of the invention (e.g. a GLP-1R/GCGR dual-agonist polypeptide).

In some embodiments, a GLP-1R agonist polypeptide of the invention (e.g. a GLP-1R/GCGR dual-agonist polypeptide) comprises a lipid is attached to an amino acid residue in the GLP-1R agonist polypeptide of the invention (e.g. a GLP-1R/GCGR dual-agonist polypeptide) by a linker, wherein the linker comprises ((O2Oc)-(O2Oc)-γE) or ((O2Oc)-(O2Oc)-γEγE) in the C- to N-terminal orientation. In some embodiments, a GLP-1R agonist polypeptide of the invention (e.g. a GLP-1R/GCGR dual-agonist polypeptide) comprises a lipid is attached to an amino acid residue in the GLP-1R agonist polypeptide of the invention (e.g. a GLP-1R/GCGR dual-agonist polypeptide) by a linker, wherein the linker consists of ((O2Oc)-(O2Oc)-γE) or ((O2Oc)-(O2Oc)-γEγE) in the C- to N-terminal orientation. In some embodiments, a GLP-1R agonist polypeptide of the invention (e.g. a GLP-1R/GCGR dual-agonist polypeptide) comprises a lipid is attached to an amino acid residue in the GLP-1R agonist polypeptide of the invention (e.g. a GLP-1R/GCGR dual-agonist polypeptide) by a linker, wherein the linker comprises or consists of ((O2Oc)-(O2Oc)-γE) in the C- to N-terminal orientation. In some embodiments, a GLP-1R agonist polypeptide of the invention (e.g. a GLP-1R/GCGR dual-agonist polypeptide) comprises a lipid is attached to an amino acid residue in the GLP-1R agonist polypeptide of the invention (e.g. a GLP-1R/GCGR dual-agonist polypeptide) by a linker, wherein the linker comprises or consists of ((O2Oc)-(O2Oc)-γEγE) in the C- to N-terminal orientation

Combinations of linker, lipid and polypeptide acylation sites are described in WO 2023/148366.

In some preferred embodiments, the amino acid residue at position 17 or 20 of the GLP-1R agonist polypeptide of the invention (e.g. the GLP-1R/GCGR dual-agonist polypeptide), or pharmaceutically acceptable salt thereof, is lipidated. In some preferred embodiments, wherein the amino acid residue at position 17 of the GLP-1R agonist polypeptide of the invention (e.g. the GLP-1R/GCGR dual-agonist polypeptide) is lipidated, the lipid is linked to the residue via a ((O2Oc)-(O2Oc)-γE) linker, and the lipid is octadecanedioic acid (C18diacid) or icosanedioic acid (C20diacid).

In some particularly preferred embodiments, wherein the amino acid residue at position 17 of the GLP-1R agonist polypeptide of the invention (e.g. the GLP-1R/GCGR dual-agonist polypeptide) is lipidated, the lipid is linked to the residue via a (O(2Oc)-O(2Oc)-γE) linker, and the lipid is octadecanedioic acid (C18diacid).

In particular, in preferred embodiments wherein the lysine at position 17 of the GLP-1R agonist polypeptide (e.g. the GLP-1R/GCGR dual-agonist polypeptide) is lipidated and/or acylated the lipid is linked to the epsilon amino group of lysine at position 17 of the GLP-1R agonist polypeptide (e.g. the GLP-1R/GCGR dual-agonist polypeptide) via a ((O2Oc)-(O2Oc)-γE) linker in the C- to N-terminal orientation, and the lipid is octadecanedioic acid (C18diacid) or icosanedioic acid (C20diacid).

In some particularly preferred embodiments wherein the lysine at position 17 of the GLP-1R agonist polypeptide (e.g. the GLP-1R/GCGR dual-agonist polypeptide) is lipidated and/or acylated the lipid is linked to the epsilon amino group of lysine at position 17 of the GLP-1R agonist polypeptide (e.g. the GLP-1R/GCGR dual-agonist polypeptide) via a ((O2Oc)-(O2Oc)-γE) linker in the C- to N-terminal orientation, and the lipid is octadecanedioic acid (C18diacid).

Combinations of linker, lipid and polypeptide acylation sites for GLP-1R agonist polypeptide of the invention (e.g. a GLP-1R/GCGR dual-agonist polypeptide) are described in WO 2023/148366, which is herein incorporated by reference in its entirety.

Exemplary GLP-1R Agonist Polypeptides (e.g. GLP-1R/GCGR Dual-Agonist Polypeptides)

In some preferred embodiments, the lipidated polypeptides of WO 2023/148366 may be used in the combination therapies of the present invention. Thus, a GLP-1R agonist polypeptide (e.g. a GLP-1R/GCGR dual-agonist polypeptide), or pharmaceutically acceptable salt thereof, may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33) or H(Aib)HGS(αMePhe)TSDVSK(αMePhe)LDSRAAKD(αMePhe)VQ(Aib)IANT-amide (SEQ ID NO: 34).

In some preferred embodiments, a GLP-1R agonist polypeptide of the invention (e.g. a GLP-1R/GCGR dual-agonist polypeptide) may comprise or consist of an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33) or H(Aib)HGS(αMePhe)TSDVSK(αMePhe)LDSRAAKD(αMePhe)VQ(Aib)IANT-amide (SEQ ID NO: 34).

In some particularly preferred embodiments, a GLP-1R agonist polypeptide of the invention (e.g. a GLP-1R/GCGR dual-agonist polypeptide) may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

In some particularly preferred embodiments, a GLP-1R agonist polypeptide of the invention (e.g. a GLP-1R/GCGR dual-agonist polypeptide) may comprise an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

In some particularly preferred embodiments, a GLP-1R agonist polypeptide of the invention (e.g. a GLP-1R/GCGR dual-agonist polypeptide) may consist of an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

More preferably, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

Most preferably, the GLP-1R agonist polypeptide of the invention (e.g. the GLP-1R/GCGR dual-agonist polypeptide, or pharmaceutically acceptable salt thereof, consists of the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

In some preferred embodiments, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C20diacid)RARDFVQWLLE(Aib)G-acid (SEQ ID NO: 37).

Most preferably, the GLP-1R agonist polypeptide of the invention (e.g. the GLP-1R/GCGR dual-agonist polypeptide, or pharmaceutically acceptable salt thereof, consists of the amino acid sequence H(Aib)QGTFTSDVSK(αMePhe)LDTK(O20c-O2Oc-γE-C20diacid)RARDFVQWLLE(Aib)G-acid (SEQ ID NO: 37).

In some embodiments, a GLP-1R agonist polypeptide of the invention (e.g. a GLP-1R/GCGR dual-agonist polypeptide) may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)HGS(αMePhe)TSDVSK(αMePhe)LDSRAAK (ε-O(2Oc)-O(2Oc)-γE-C18diacid)D(αMePhe)VQ(Aib)IANT-amide (SEQ ID NO: 36).

In some embodiments, a GLP-1R agonist polypeptide of the invention (e.g. a GLP-1R/GCGR dual-agonist polypeptide) may comprise or consist of an amino acid sequence having at least 90%, at least 95%, at least 97%, at least 98%, at least 99% or more, up to 100% identity to H(Aib)HGS(αMePhe)TSDVSK(αMePhe)LDSRAAK(ε-O(2Oc)-O(2Oc)-γE-C18diacid)D(αMePhe)VQ(Aib)IANT-amide (SEQ ID NO: 36).

In some embodiments, a GLP-1R agonist polypeptide of the invention (e.g. a GLP-1R/GCGR dual-agonist polypeptide) may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)HGS(αMePhe)TSDVSK(αMePhe)LDSRAAK(E-O(2Oc)-O(2Oc)-γE-γE-C20diacid)D(αMePhe)VQ(Aib)IANT-amide (SEQ ID NO: 38).

In some embodiments, a GLP-1R agonist polypeptide of the invention (e.g. a GLP-1R/GCGR dual-agonist polypeptide) may comprise or consist of an amino acid sequence having at least 90%, at least 95%, at least 97%, at least 98%, at least 99% or more, up to 100% identity to H(Aib)HGS(αMePhe)TSDVSK(αMePhe)LDSRAAK(ε-O(2Oc)-O(2Oc)-γE-γE-C20diacid) D(αMePhe)VQ(Aib)IANT-amide (SEQ ID NO: 38).

### Pharmacokinetics

A GLP-1R agonist polypeptide (e.g. a GLP-1R/GCGR dual-agonist polypeptide) used in the invention may exhibit favourable pharmacokinetic properties as compared to cotadutide. For example, the GLP-1R agonist polypeptides (e.g. the GLP-1R/GCGR dual-agonist polypeptides) may have an extended half-life as compared to cotadutide.

In some aspects, a GLP-1R agonist polypeptide (e.g. a GLP-1R/GCGR dual-agonist polypeptides) has at least 50%, at least 60%, at least 70%, at least 75%, at least 80%, at least 85%, at least 90%, at least 95, or 100% of intact GLP-1R agonist polypeptide (e.g. GLP-1R/GCGR dual-agonist polypeptide) remaining after incubation with a protease at 37° C. for 5 min, 10 min, 15 min, 30 min, 2 hr, 4 hr or 24 hr. In some aspects, the protease is selected from the group consisting of neprilysin, pepsin, pancreatin, simulated gastric fluid with pepsin, and simulated intestinal fluid with pancreatin.

In some aspects, the GLP-1R agonist polypeptide (e.g. the GLP-1R/GCGR dual-agonist polypeptide) has a half-life in cynomolgus monkeys after intravenous administration of at least 45 hours, at least 50 hours, at least 60 hours, at least 70 hours, at least 80 hours, at least 90 hours, at least 100 hours, at least 110 hours, at least 120 hours, or about 130 hours.

In some aspects, the GLP-1R agonist polypeptide (e.g. the GLP-1R/GCGR dual-agonist polypeptide) has an s.c. bioavailability in cynomolgus monkeys of at least 75%, at least 80%, at least 90%, or about 95%.

In preferred embodiments, the GLP-1R agonist polypeptides (e.g. the GLP-1R/GCGR dual-agonist polypeptides) are soluble at concentrations required for therapeutic efficacy.

### Combination Therapy

As exemplified herein, the present inventors have demonstrated for the first time that combining a particular class of AMYR agonists with GLP-1R agonists provides advantageous properties. In particular, as exemplified herein, combining AMYR agonists which has selectivity for AMYR as compared with CTR with GLP-1R agonists results in fat-mass specific weight loss and reduced aversion in pre-clinical rat models. Accordingly, the present inventors provide for the first time a combination AMYR agonist and GLP1-R agonist treatment which promotes fat-mass loss and preserves lean-mass without compromising tolerability, making this a clinically attractive treatment paradigm. As further demonstrated herein, this combination treatment has the potential to provide synergistic effects beyond those observed using AMYR agonists and GLP1-R agonists as monotherapies. The present invention is based, at least in part on these findings.

Accordingly, provided herein is a combination therapy comprising (a) an AMYR agonist which has selectivity for AMYR as compared with CTR, and (b) a GLP-1R agonist.

Examples of AMYR agonists with selectivity for AMYR as compared with CTR suitable for use in a combination therapy of the invention are described herein. Similarly, examples of GLP-1R agonists suitable for use in a combination therapy of the invention are described herein. Any AMYR agonists with selectivity for AMYR as compared with CTR as described herein may be combined with any GLP-1R agonist as described herein.

In some preferred embodiments, the AMYR agonist with selectivity for AMYR as compared with CTR is combined with a GLP-1R/GCGR dual-agonist.

In some preferred embodiments, the AMYR agonist with selectivity for AMYR as compared with CTR is an AMYR agonist polypeptide and GLP-1R agonist is a GLP-1R agonist polypeptide, as described herein.

In some particularly preferred embodiments, the AMYR agonist with selectivity for AMYR as compared with CTR is an AMYR agonist polypeptide and GLP-1R agonist is a GLP-1R/GCGR dual-agonist polypeptide, as described herein

Typically the AMYR agonist polypeptide and/or the GLP-1R agonist polypeptide (e.g. the GLP-1R/GCGR dual-agonist polypeptide) are lipidated. Preferably, both the AMYR agonist polypeptide and the GLP-1R agonist polypeptide (e.g. the GLP-1R/GCGR dual-agonist polypeptide) are lipidated.

By way of non-limiting example, in some preferred embodiments, a combination therapy comprises or consists of (a) an AMYR agonist polypeptide, or pharmaceutically acceptable salt thereof comprising an amino acid sequence having at least 70%, at least 80%, at least 90%, at least 95%, at least 96%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26); and (b) a GLP-1R agonist polypeptide, or pharmaceutically acceptable salt thereof comprising an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

By way of further non-limiting example, in some preferred embodiments, a combination therapy comprises or consists of (a) an AMYR agonist polypeptide consisting of an amino acid sequence having at least 70%, at least 80%, at least 90%, at least 95%, at least 96%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26), or pharmaceutically acceptable salt thereof; and (b) a GLP-1R agonist polypeptide consisting of an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33), or pharmaceutically acceptable salt thereof.

By way of further non-limiting example, in some preferred embodiments, a combination therapy comprises or consists of (a) an AMYR agonist polypeptide, or pharmaceutically acceptable salt thereof comprising an amino acid sequence having at least 90%, at least 95%, at least 96%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26); and (b) a GLP-1R agonist polypeptide, or pharmaceutically acceptable salt thereof comprising an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

By way of further non-limiting example, in some preferred embodiments, a combination therapy comprises or consists of (a) an AMYR agonist polypeptide consisting of an amino acid sequence having at least 90%, at least 95%, at least 96%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26), or pharmaceutically acceptable salt thereof; and (b) a GLP-1R agonist polypeptide consisting of an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33), or pharmaceutically acceptable salt thereof.

By way of further non-limiting example, in some particularly preferred embodiments, a combination therapy comprises or consists of (a) an AMYR agonist polypeptide, or pharmaceutically acceptable salt thereof, comprising the amino acid sequence K (γE-γE-C18diacid)[CNTATC]ATQRLANFLRHSSNN(αMePhe)GPILPPTEVGSNTY-amide (SEQ ID NO: 11); and (b) a GLP-1R agonist polypeptide, or pharmaceutically acceptable salt thereof, comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

By way of further non-limiting example, in some particularly preferred embodiments, a combination therapy comprises or consists of (a) an AMYR agonist polypeptide, consisting of the amino acid sequence K (γE-γE-C18diacid)[CNTATC]ATQRLANFLRHSSNN(αMePhe)GPILPPTEVGSNTY-amide (SEQ ID NO: 11), or pharmaceutically acceptable salt thereof; and (b) a GLP-1R agonist polypeptide consisting of the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof.

As described herein, the GLP-1R agonist may be a GLP-1R/GCGR dual-agonist. Alternatively, a combination therapy of the invention may comprise a separate GCGR agonist. In other words, a combination therapy of the invention may comprise or consist of (a) an AMYR agonist; (b) a GLP-1R agonist; and (c) GCGR agonist; wherein each of (a)-(c) are separate agents. In such embodiments, each of (a)-(c) may be independently selected a polypeptide, small molecule drug, antibody, antibody-drug conjugate, or aptamer; or a pharmaceutically acceptable salt thereof. In some preferred embodiments, each of (a)-(c) is an agonist polypeptide. Any AMYR agonist and/or GLP-1R agonist described herein, particularly any AMYR agonist polypeptide and/or GLP-1R agonist polypeptide described herein, may be used in such embodiments.

A combination therapy of the invention may comprise one or more additional active agent useful in the treatment of a disease or disorder as described herein. By way of non-limiting example, a combination therapy may further comprise a gastric inhibitory polypeptide (GIP) agonist. In some embodiments, a GLP-1R agonist of the invention (e.g. a GLP-1R/GCGR dual-agonist) may further comprise GIP agonist activity. Alternatively, a separate GIP agonist may be combined with an AMYR agonist and a GLP-1R agonist of the invention (e.g. a GLP-1R/GCGR dual-agonist).

As exemplified herein, a combination therapy of the invention can provide surprising benefits compared with monotherapy using an AMYR agonist or a GLP-1R agonist (e.g. a GLP-1R/GCGR dual-agonist).

A combination therapy of the invention may provide increased body weight loss as described herein. Typically, a combination therapy of the invention provides increased body weight loss (in absolute or % terms) compared with monotherapy using either the AMYR agonist or a GLP-1R agonist (e.g. a GLP-1R/GCGR dual-agonist) used in the combination thereof. By way of non-limiting example, a combination therapy of the invention may reduce body weight in DIO rats by more than 5% compared with DIO mice treated with the AMYR agonist or the GLP-1R agonist (e.g. a GLP-1R/GCGR dual-agonist) monotherapy, as exemplified herein. A combination therapy of the invention may reduce body weight in DIO rats by more than 10% compared with DIO mice treated with the AMYR agonist or the GLP-1R agonist (e.g. a GLP-1R/GCGR dual-agonist) monotherapy, as exemplified herein. By way of further non-limiting example, a combination therapy of the invention may reduce body weight in DIO rats to within 5% of the body weight of a non-obese control rat, as exemplified herein. A combination therapy of the invention may reduce body weight in DIO rats to within 2% of the body weight of a non-obese control rat, as exemplified herein.

Alternatively or in addition, a combination therapy of the invention may provide fat-mass specific body weight loss as described herein. By way of non-limiting example, a combination therapy of the invention may reduce the % fat mass in DIO rats to within 5% of the fat mass of a non-obese control rat, as exemplified herein. A combination therapy of the invention may reduce the % fat mass in DIO rats to within 2% of the fat mass of a non-obese control rat, as described herein. Fat-mass specific body weight loss may comprise reducing the absolute amount of fat-mass specific body weight in a subject (e.g., in grams), and/or reducing the proportion of the fat-specific body weight component of total body weight (e.g., as a percentage of total body weight). In some embodiments, the combination therapy of the invention preferentially reduces fat-mass specific body weight as compared to lean mass-specific (e.g. muscle) body weight (e.g., preserving lean-mass specific body weight while reducing overall body weight). A combination therapy of the invention may result in increased fat mass specific body weight loss (and/or preservation of lean mass) when compared to matched total body weight loss levels when the same or a different subject are treated with a combination of a DACRA and a GLP-1R agonist (e.g. a GLP-1R/GCGR dual-agonist), and/or compared with monotherapy using either the AMYR agonist or a GLP-1R agonist (e.g. a GLP-1R/GCGR dual-agonist) used in the combination thereof, or a monotherapy using a different anti-obesity drug, such as a DACRA (e.g., cagrilintide).

Alternatively or in addition, a combination therapy of the invention may provide no worsening of aversion to saccharin (i.e. increase saccharin preference) in DIO rats, as described herein. By way of non-limiting example, a combination therapy of the invention may increase saccharin preference in DIO rats to more than 20%, as exemplified herein. A combination therapy of the invention may increase saccharin preference in DIO rats to more than 30%, as exemplified herein. Said reduced saccharin aversion (i.e. increase saccharin preference) in DIO rats may be compared with DIO rats treated with a combination of a dual amylin and calcitonin receptor agonist (DACRA) and a GLP-1R agonist (e.g. a GLP-1R/GCGR dual-agonist), as described herein. As used herein, the term “DACRA” refers to an agonist which can equally activate both AMYR and CTR, or which has less than 2-fold selectivity to AMYR as compared to CTR, as described herein.

Alternatively or in addition, a combination therapy of the invention may reduce nausea and/or vomiting. Said reduction in nausea and/or vomiting may be compared with nausea and/or vomiting experienced when the same or a different subject are treated with a combination of a DACRA and a GLP-1R agonist (e.g. a GLP-1R/GCGR dual-agonist), and/or compared with nausea and/or vomiting experienced when the same or a different subject are treated with monotherapy using either the AMYR agonist or a GLP-1R agonist (e.g. a GLP-1R/GCGR dual-agonist) used in the combination thereof, or a monotherapy using a different anti-obesity drug, such as a DACRA (e.g., cagrilintide or petrelintide). Thus, a combination therapy according to the present invention may reduce the need for the subject to be administered an anti-emetic medication as compared to a therapeutically effective dose of a combination of a DACRA and a GLP-1R agonist (e.g. a GLP-1R/GCGR dual-agonist), and/or compared with monotherapy treatment using either the AMYR agonist or a GLP-1R agonist (e.g. a GLP-1R/GCGR dual-agonist) used in the combination thereof, or monotherapy using a different anti-obesity drug, such as a DACRA (e.g., cagrilintide or petrelintide). Suitable anti-emetic medications will be familiar to a person skilled in the art. Thus, a combination therapy according to the present invention may allow the subject to be administered a reduced dose of an anti-emetic medication as compared to a therapeutically effective dose of a different anti-obesity drug combination or monotherapy. Alternatively or in addition, a combination therapy according to the present invention may allow the subject to be administered an anti-emetic medication at a reduced dosing frequency as compared to a therapeutically effective dose of a different anti-obesity drug combination or monotherapy. A combination therapy according to the present invention may eliminate the need for a subject to be administered an anti-emetic medication, i.e., the subject is not administered an anti-emetic medication.

### Add-on Therapy

As exemplified herein, AMYR agonists, particularly those with selectivity for AMYR as compared with CTR, can drive additional weight loss when added to existing therapy with a GLP-1R agonist in pre-clinical rat models of type 2 diabetes. Accordingly, the present inventors provide an AMYR agonist as an add-on or additional therapy in subjects who are overweight (≥27 kg/m²) or obese (≥30 kg/m²) and/or have type 2 diabetes, and who are on a stable dose of a GLP-1R agonist. Particularly, the present inventors provide an AMYR agonist as an add-on or additional therapy in subjects who are overweight (≥27 kg/m²) or obese (≥30 kg/m²) and have type 2 diabetes, and who are on a stable dose of a GLP-1R agonist. By a “stable dose of a GLP-1R agonist”, it will be understood that the subject is on a stable diabetes maintenance dose of said GLP-1R agonist for at least two months, preferably at least three months, more preferably at least six months at the time the add-on therapy with an AMYR agonist commences.

Accordingly, also provided herein is a method of inhibiting or reducing weight gain, promoting weight loss, reducing food intake, increasing satiety, and/or reducing excess body weight in a subject, the method comprising administering the AMYR agonist, or pharmaceutically acceptable salts thereof, to the subject, wherein said subject: (i) is overweight or obese and has type 2 diabetes; and (ii) who is being treated with a GLP-1R agonist at the time treatment with the AMYR agonist commences, and optionally who continues treatment with a GLP-1R agonist after treatment with the AMYR agonist commences.

Said weight loss may be fat-mass specific, as described herein. In addition, said method may improve glycaemic and/or metabolic control in a subject, also as described herein.

Examples of AMYR agonists, particularly AMYR agonists with selectivity for AMYR as compared with CTR, suitable for use in such methods are described herein. Similarly, examples of GLP-1R agonists of which a subject may be receiving a stable dose prior to such a method are described herein. Any AMYR agonists with selectivity for AMYR as compared with CTR as described herein may be combined with any GLP-1R agonist as described herein.

The AMYR agonist with selectivity for AMYR as compared with CTR may be that of SEQ ID NO: 11. Alternatively or in addition, the GLP-1R agonist may be selected from the group consisting of Exenatide (Byetta), Liraglutide (Victoza), Dulaglutide (Trulicity), Semaglutide (Ozempic, Rybelsus), and Lixisenatide (Adlyxin).

Also provided is an AMYR agonist, or pharmaceutically acceptable salt thereof, for use in a method of inhibiting or reducing weight gain, promoting weight loss, reducing food intake, increasing satiety, and/or reducing excess body weight in a subject, the method comprising administering the AMYR agonist, or pharmaceutically acceptable salt thereof, wherein said subject: (i) is overweight or obese and has type 2 diabetes; and (ii) who is being treated with a GLP-1R agonist at the time treatment with the AMYR agonist commences, and optionally who continues treatment with a GLP-1R agonist after treatment with the AMYR agonist commences.

### Pharmaceutical Compositions

In aspects of the invention, an AMYR agonist and a GLP-1R agonist (e.g. a GLP-1R/GCGR dual-agonist) of the invention are provided in a pharmaceutical composition. As described herein, said AMYR agonist and/or GLP-1R agonist (e.g. a GLP-1R/GCGR dual-agonist) are typically agonist polypeptides.

Further provided are compositions, e.g., pharmaceutical compositions, that contain an effective amount of an AMYR agonist and a GLP-1R agonist (e.g. a GLP-1R/GCGR dual-agonist) of the invention as provided herein. In some embodiments, the compositions are formulated for the treatment of obesity, an obesity-related condition or a metabolic disease as described herein, e.g., obesity, type 2 diabetes, and/or MASH.

The pharmaceutical compositions of the invention may comprise one or more excipient(s), carrier, buffer, stabilizer or other materials well known to those skilled in the art. Such materials should be non-toxic and should not interfere with the efficacy of the active ingredient. The precise nature of the carrier or other material will depend on the route of administration. Pharmaceutically acceptable excipients are known in the art, see for instance Remington's Pharmaceutical Sciences (by Joseph P. Remington, 18th ed., Mack Publishing Co., Easton, PA), which is incorporated herein in its entirety.

Composition can be in a variety of forms, including, but not limited to an aqueous solution, an emulsion, a gel, a suspension, lyophilized form, or any other form known in the art. In addition, the composition can contain pharmaceutically acceptable additives including, for example, diluents, binders, stabilizers, and preservatives. Once formulated, compositions of the invention can be administered directly to the subject.

For injectable formulations, e.g., for subcutaneous injection, the active ingredient will be in the form of a parenterally acceptable aqueous solution which is pyrogen-free and has suitable pH, isotonicity and stability.

An AMYR agonist and a GLP-1R agonist (e.g. a GLP-1R/GCGR dual-agonist) as described herein may be formulated in liquid, semi-solid or solid forms depending on the physicochemical properties of the molecule and the route of delivery. Formulations may include excipients, or combinations of excipients, for example: sugars, amino acids and surfactants. Liquid formulations may include a wide range of antibody concentrations and pH. Solid formulations may be produced by lyophilisation, spray drying, or drying by supercritical fluid technology, for example. Treatment may be given by injection (for example, subcutaneously, or intra-venously. The treatment may be administered orally. The route of administration can be determined by the physicochemical characteristics of the treatment, by special considerations for the disease or by the requirement to optimize efficacy or to minimize side-effects.

Preferred routes of administering pharmaceutical compositions of the present invention are subcutaneous and/or oral administration.

In some aspects, the pharmaceutical composition is a solid composition. In some aspects, the pharmaceutical composition is a liquid composition.

A composition may be administered alone or in combination with other treatments, either simultaneously or sequentially dependent upon the condition to be treated.

An AMYR agonist and a GLP-1R agonist (e.g. a GLP-1R/GCGR dual-agonist) may be used as part of a combination therapy in conjunction with an additional medicinal component. Combination treatments may be used to provide significant synergistic effects, particularly the combination of an AMYR agonist and a GLP-1R agonist (e.g. a GLP-1R/GCGR dual-agonist) with one or more other drugs. An AMYR agonist and a GLP-1R agonist (e.g. a GLP-1R/GCGR dual-agonist) may be administered concurrently or sequentially or as a combined preparation with another therapeutic agent or agents, for the treatment of one or more of the conditions listed herein.

### Therapeutic Methods

The present invention encompasses therapies which involve administering an AMYR agonist and a GLP-1R agonist (e.g. a GLP-1R/GCGR dual-agonist) of the invention to an animal, in particular a mammal, for instance a human, for preventing, treating, or ameliorating symptoms associated with a disease, disorder, or infection.

Accordingly, the invention provides an AMYR agonist and GLP-1R agonist (e.g. a GLP-1R/GCGR dual-agonist) or pharmaceutically acceptable salts thereof of the invention for use in therapy, for example in a method of treating and/or preventing a disease or disorder.

Also provided is a method of treating and/or preventing a disease or disorder comprising administering to a subject or patient in need thereof a therapeutically effective amount of the AMYR agonist and the GLP-1R agonist (e.g. a GLP-1R/GCGR dual-agonist) (and optionally, any additional active agents, such as a GIP agonist as described herein), or pharmaceutically acceptable salts thereof, of the invention.

The use or method may comprise administering a therapeutically effective schedule that has less frequent doses of the AMYR agonist and the GLP-1R agonist (e.g. a GLP-1R/GCGR dual-agonist) than the therapeutically effective dosing schedule of pramlintide or cotadutide.

It will be understood that the AMYR agonist and the GLP-1R agonist (e.g. a GLP-1R/GCGR dual-agonist) may be used in the treatment and/or prevention of obesity, metabolic diseases (such as diabetes, e.g. type 1 or type 2 diabetes), and/or obesity-related conditions. The invention also provides an AMYR agonist and a GLP-1R agonist (e.g. a GLP-1R/GCGR dual-agonist) for use in such methods.

In preferred embodiments, the AMYR agonist and the GLP-1R agonist (e.g. a GLP-1R/GCGR dual-agonist) may be used in a method of treating and/or preventing obesity. Said method may comprise administering a therapeutically effective amount of the AMYR agonist and the GLP-1R agonist (e.g. a GLP-1R/GCGR dual-agonist) to the subject. The invention also provides an AMYR agonist and a GLP-1R agonist (e.g. a GLP-1R/GCGR dual-agonist) for use in such a method.

In some embodiments, the AMYR agonist and the GLP-1R agonist (e.g. a GLP-1R/GCGR dual-agonist) may be used in a method of treating and/or preventing an obesity-related condition in a subject. In some embodiments, the obesity-related condition is overweight, morbid obesity, obesity prior to surgery, obesity-linked inflammation, obesity-linked gallbladder disease, sleep apnoea and respiratory problems, hyperlipidaemia, degeneration of cartilage, osteoarthritis, or reproductive health complications of obesity or overweight such as infertility in a subject. The invention also provides an AMYR agonist and a GLP-1R agonist (e.g. a GLP-1R/GCGR dual-agonist) for use in such a method.

In some embodiments, the AMYR agonist and the GLP-1R agonist (e.g. a GLP-1R/GCGR dual-agonist) may be used in a method of treating and/or preventing a metabolic disease. Said metabolic disease may be diabetes, type 1 diabetes, type 2 diabetes, gestational diabetes, pre-diabetes, insulin resistance, impaired glucose tolerance (IGI), disease states associated with elevated blood glucose levels, metabolic disease including metabolic syndrome, or hyperglycaemia, e.g. abnormal postprandial hyperglycemia. The invention also provides an AMYR agonist and a GLP-1R agonist (e.g. a GLP-1R/GCGR dual-agonist) for use in such a method. In preferred embodiments, the polypeptides of the invention are used for the treatment of type 1 diabetes or type 2 diabetes. In particularly preferred embodiments, the polypeptides of the invention are used for the treatment of type 2 diabetes.

In some embodiments, the AMYR agonist and the GLP-1R agonist (e.g. a GLP-1R/GCGR dual-agonist) may be used in a method of treating and/or preventing metabolic dysfunction associated steatohepatitis (MASH).

The treatment and/or prevention of a disease or disorder using an AMYR agonist and a GLP-1R agonist (e.g. a GLP-1R/GCGR dual-agonist) may further comprise treating, improving, and/or protecting liver and/or kidney function in the subject. Treating, improving, and/or protecting liver function may comprise reducing steatohepatitis and/or fibrosis in the liver.

Also provided is a method of inhibiting or reducing weight gain, promoting weight loss, reducing food intake, increasing satiety, and/or reducing excess body weight, the method comprising an AMYR agonist and a GLP-1R agonist (e.g. a GLP-1R/GCGR dual-agonist) of the invention to the subject. The invention also provides an AMYR agonist and a GLP-1R agonist (e.g. a GLP-1R/GCGR dual-agonist) for use in such a method. Said method may be therapeutic, e.g. when the subject is obese, suffers from an obesity-related condition and/or a metabolic disease. Alternatively, said method may be cosmetic (non-therapeutic), e.g. when the patient wishes to lose weight for aesthetic reasons.

Thus, the invention also provides a cosmetic use of an AMYR agonist and a GLP-1R agonist (e.g. a GLP-1R/GCGR dual-agonist) of the invention to inhibit or reduce weight gain, promote weight loss, reduce food intake, increase satiety, and/or reduce excess body weight in a subject.

There is also provided a method of reducing fat mass-specific body weight, the method comprising administering the polypeptides of the invention to the subject. In the context of the invention, “reducing fat mass-specific body weight” may comprise reducing the absolute amount of fat-specific body weight in a subject (e.g., in grams), and/or reducing the proportion of the fat-specific body weight component of total body weight (e.g., as a percentage of total body weight). In some embodiments, the method of treatment preferentially reduces fat-mass specific body weight as compared to lean mass-specific (e.g. muscle) body weight (e.g., preserving lean-mass specific body weight while reducing overall body weight). The invention also provides an AMYR agonist and a GLP-1R agonist (e.g. a GLP-1R/GCGR dual-agonist) for use in such a method. Said method may be therapeutic, e.g. when the subject is obese, suffers from an obesity-related condition and/or a metabolic disease. Alternatively, said method may be cosmetic (non-therapeutic), e.g. when the patient wishes to lose weight for aesthetic reasons.

Thus, the invention also provides a cosmetic use of an AMYR agonist and a GLP-1R agonist (e.g. a GLP-1R/GCGR dual-agonist) of the invention to reduce fat-mass specific body weight in a subject.

Also provided herein is a method of treating, improving, or protecting liver and/or kidney function in a subject, the method comprising administering an AMYR agonist and a GLP-1R agonist (e.g. a GLP-1R/GCGR dual-agonist) to the subject. The invention also provides an AMYR agonist and a GLP-1R agonist (e.g. a GLP-1R/GCGR dual-agonist) for use in such a method.

Also provided herein is a method of improving glycemic and/or metabolic control in a subject, the method comprising administering an AMYR agonist and a GLP-1R agonist (e.g. a GLP-1R/GCGR dual-agonist) of the invention to the subject. The invention also provides an AMYR agonist and a GLP-1R agonist (e.g. a GLP-1R/GCGR dual-agonist) for use in such a method.

Improving glycemic and/or metabolic control may comprise one or more of: increasing insulin secretion, delaying gastric emptying, increasing mitochondria function, inhibiting de novo lipogenesis, decreasing HbAlc, enhancing fatty oxidation, decreasing hepatic mitochondrial oxidative stress, decreasing steatosis, decreases fibrosis, decreasing glycogen synthesis, increasing gluconeogenesis, improving glycemic control, reducing or reversing fibrosis (e.g., liver fibrosis), reducing steatohepatitis, and/or reducing risk of death due to cirrhosis, hepatocellular carcinoma, and/or cardiorenal disease in a subject in need thereof. Improving glycemic and/or metabolic control may comprise reducing endogenous leptin levels and/or improving or restoring leptin sensitivity in a subject.

The invention also provides a method of delaying gastric emptying in a subject, the method comprising administering an AMYR agonist and a GLP-1R agonist (e.g. a GLP-1R/GCGR dual-agonist) of the invention to the subject. The invention also provides an AMYR agonist and a GLP-1R agonist (e.g. a GLP-1R/GCGR dual-agonist) for use in such a method.

Also provided herein is an AMYR agonist and a GLP-1R agonist (e.g. a GLP-1R/GCGR dual-agonist) of the invention for use in the manufacture of a medicament. Said medicament may be for use in the treatment and/or prevention of any disease or disorder as described herein.

The route of administration of an AMYR agonist and a GLP-1R agonist (e.g. a GLP-1R/GCGR dual-agonist), or pharmaceutical compositions thereof, can be, for example, oral, parenteral, by inhalation or topical. In preferred embodiments, the AMYR agonist and the GLP-1R agonist (e.g. the GLP-1R/GCGR dual-agonist) or pharmaceutical compositions thereof are administered by parenteral administration to a subject or patient. The term “parenteral” as used herein includes, e.g., intravenous, intraarterial, intraperitoneal, intramuscular, subcutaneous, rectal, or vaginal administration. In preferred embodiments, the AMYR agonist and the GLP-1R agonist (e.g. the GLP-1R/GCGR dual-agonist) or pharmaceutical compositions thereof are administered by injection, such as by intravenous, subcutaneous or intramuscular injection, to a subject or patient. In particularly preferred embodiments, the AMYR agonist and the GLP-1R agonist (e.g. the GLP-1R/GCGR dual-agonist) or pharmaceutical compositions thereof are administered orally. In other particularly preferred embodiments, the AMYR agonist and the GLP-1R agonist (e.g. the GLP-1R/GCGR dual-agonist) or pharmaceutical compositions thereof are administered by subcutaneous injection. Oral administration, or administration by injection, such as by subcutaneous injection, offers the advantage of better comfort for the subject or patient and the opportunity to administer to a subject or patient outside of a hospital setting. In some embodiments, the AMYR agonist and the GLP-1R agonist (e.g. the GLP-1R/GCGR dual-agonist) or pharmaceutical compositions thereof are administered by self-administration.

In some embodiments the subject or patient is a mammal, in particular a human.

In some embodiments, the AMYR agonist and the GLP-1R agonist (e.g. the GLP-1R/GCGR dual-agonist) or pharmaceutical compositions thereof are administered to the subject in combination with insulin.

In some aspects, the AMYR agonist and the GLP-1R agonist (e.g. the GLP-1R/GCGR dual-agonist) or pharmaceutical compositions thereof are administered once a week, every two weeks, every three weeks, or every four weeks. In some aspects, the AMYR agonist and the GLP-1R agonist (e.g. the GLP-1R/GCGR dual-agonist) or pharmaceutical compositions thereof are administered about once every 4, 5, 6, 7, 8, 9, 10, 11, 12, 13, or 14 days. In a preferred embodiment, the AMYR agonist and the GLP-1R agonist (e.g. the GLP-1R/GCGR dual-agonist) or pharmaceutical compositions thereof are administered about once a week.

The AMYR agonist and the GLP-1R agonist (e.g. the GLP-1R/GCGR dual-agonist) or pharmaceutical compositions thereof may be administered simultaneously. Alternatively, the AMYR agonist and the GLP-1R agonist (e.g. the GLP-1R/GCGR dual-agonist) or pharmaceutical compositions thereof may be administered sequentially.

The AMYR agonist and the GLP-1R agonist (e.g. the GLP-1R/GCGR dual-agonist) or pharmaceutical compositions thereof may be administered using a dual-chamber device, as described herein.

Any AMYR agonist which has selectivity for AMYR as compared with CTR, and any GLP-1R agonist as described herein may be used for therapy, in a method of treatment or in the manufacture of a medicament according to the present invention. Preferably, the AMYR agonist with selectivity for AMYR as compared with CTR is an AMYR agonist polypeptide and GLP-1R agonist is a GLP-1R agonist polypeptide, as described herein In some preferred embodiments, the AMYR agonist with selectivity for AMYR as compared with CTR is combined with a GLP-1R/GCGR dual-agonist, as described herein. Thus, in some particularly preferred embodiments, the AMYR agonist with selectivity for AMYR as compared with CTR is an AMYR agonist polypeptide and GLP-1R agonist is a GLP-1R/GCGR dual-agonist polypeptide, as described herein. By way of non-limiting example, an AMYR agonist polypeptide, or pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11); and a GLP-1R agonist polypeptide, or pharmaceutically acceptable salt thereof, comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35) may be used in a therapy as described herein. By way of further non-limiting example, an AMYR agonist polypeptide, consisting of amino the acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof; and a GLP-1R agonist polypeptide consisting of the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof may be used in a therapy as described herein.

Alternatively, any AMYR agonist which has selectivity for AMYR as compared with CTR, and any GLP-1R agonist as described herein may be used for therapy, in a method of treatment or in the manufacture of a medicament according to the present invention in combination with a GCGR agonist. Thus, a method of the invention may comprise administering an AMYR agonist which has selectivity for AMYR as compared with CTR, and a GLP-1R agonist as described herein to a subject, and further comprise administering to the subject a GCGR agonist.

### Methods of Production

The polypeptides of the invention, particularly the AMYR agonist polypeptides and/or the GLP-1R agonist polypeptides may be produced by any method known in the art.

The production of polypeptides such as AMYR agonist polypeptides, and GLP-1/glucagon agonist polypeptides, is well known in the art. The AMYR agonist polypeptides and/or GLP-1/glucagon agonist polypeptides of the invention can thus be produced by chemical synthesis, e.g. solid phase polypeptide synthesis using t-Boc or Fmoc chemistry, or other well-established techniques as described by Merrifield (1963, J. Am. Chem. Soc. 85:2149-2154). Solid phase peptide synthesis can be accomplished, e.g., by using automated synthesizers, using standard reagents, e.g., as explained in Example 1 of WO 2014/091316, which is herein incorporated by reference in its entirety.

Alternatively, AMYR agonist polypeptides and/or GLP-1/glucagon agonist polypeptides provided herein can be produced recombinantly using a convenient vector/host cell combination as would be well known to the person of ordinary skill in the art, e.g., by recombinant expression of a nucleic acid molecule encoding a fusion polypeptide in a host cell. Generally, a polynucleotide sequence encoding the polypeptide is inserted into an appropriate expression vehicle, e.g., a vector which contains the necessary elements for the transcription and translation of the inserted coding sequence. The nucleic acid encoding the polypeptide is inserted into the vector in proper reading frame. The expression vector is then transfected into a suitable host cell which will express the polypeptide. Suitable host cells include without limitation bacteria, yeast, or mammalian cells. A variety of commercially-available host-expression vector systems can be utilized to express the polypeptides described herein.

Following synthesis, the AMYR agonist polypeptides and/or GLP-1/glucagon agonist polypeptides may optionally be isolated or purified.

### Articles of Manufacture and Kits

The present invention provides an article of manufacture comprising AMYR agonist polypeptides and/or GLP-1/glucagon agonist polypeptides or pharmaceutical compositions of the invention.

The present invention further provides a kit comprising AMYR agonist polypeptides and/or GLP-1/glucagon agonist polypeptides or pharmaceutical compositions of the invention. The kit may comprise a package containing the AMYR agonist polypeptides and/or GLP-1/glucagon agonist polypeptides or pharmaceutical composition, optionally with instructions.

In some embodiments, in said kit or article of manufacture, the AMYR agonist polypeptides and/or GLP-1/glucagon agonist polypeptides or pharmaceutical compositions of the invention are formulated in single dose vials or a container closure system (e.g. pre-filled syringe). Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, in said kit or article of manufacture, the AMYR agonist polypeptides and/or GLP-1/glucagon agonist polypeptides) or pharmaceutical compositions of the invention are formulated in a dual-chamber device, e.g., a dual-chamber syringe (DCS). Typically, such devices comprise a pre-filled double chamber mechanism for simultaneous administration of two active ingredients, in which a first active ingredient is formulated in a first syringe chamber or cartridge, and a second active ingredient is formulated in a second syringe chamber or cartridge. The chambers may be separated by a middle plunger that on activation allows the two active ingredients to mix in one drug chamber immediately before administration. The main advantages of such pre-filled devices are the increase of dose accuracy, the lower risk of microbial contamination, and the reduction of the procedure time and handling steps required. Some DCSs incorporate a parallel post-injection safety sheath chamber for enclosing a sharpened needle tip. These syringes are developed as a protection of the inadvertent contact with the needles after the syringe has been used, which can be extremely important in the reduction of needle-stick injuries and contact with blood-transmitted diseases such as hepatitis or acquired immune deficiency syndrome.

### Brief Description of Sequence Listing

Where present, a square bracket [ ] between the two cysteine residues (Cys2 and Cys7) indicate the presence of an intramolecular disulphide bridge.

γE or yE = gamma-glutamate
SEQ ID NO: 1
exemplary amino acid sequence for hRAMP1
SEQ ID NO: 2
exemplary amino acid sequence for hRAMP2
SEQ ID NO: 3
exemplary amino acid sequence for hRAMP3
SEQ ID NO: 4
exemplary amino acid sequence for hCTR
SEQ ID NO: 5
pramlintide amino acid sequence
SEQ ID NO: 6
C18diacid-γE-[CNTATC]ATQRLAEFLRHSSNN(αMePhe)GPILPPTNVGSNTY-amide
SEQ ID NO: 7
K(γE-γE-C18diacid)[CNTATC]ATQRLAEFLRHSSNN(αMePhe)GPILPPTNVGSNTY-amide
SEQ ID NO: 8
K(γE-C18diacid)K[CNTATC]ATQRLAEFLRHSSNN(αMePhe)GPILPPTNVGSNTY-amide);
SEQ ID NO: 9
K(γE-C18diacid)K[CNTATC]ATQRLANFLRHSSNN(αMePhe)GPILPPTEVGSNTY-amide
SEQ ID NO: 10
K(O2Oc-O2Oc-γE-18diacid)[CNTATC]ATQRLANFLRHSSNN(αMePhe)GPILPPTEVGSNTY-amide
SEQ ID NO: 11
K(γE-γE-C18diacid)[CNTATC]ATQRLANFLRHSSNN(αMePhe)GPILPPTEVGSNTY-amide
SEQ ID NO: 12
K(γE-C18diacid)[CNTATC]ATQRLANFLRHSSNN(αMePhe)GPILPPTNVGSNTY-amide
SEQ ID NO: 13
K(γE-γE-C18diacid)[CNTATC]ATQRLANFLRHSSNN(αMePhe)GPILPPTNVGSNTY-amide
SEQ ID NO: 14
K(γE-γE-C18diacid)[CNTATC]ATQRLANFLVHSSNN(αMePhe)GPILPPTNVGSRTY-amide
SEQ ID NO: 15
K(γE-γE-C18diacid)[CNTATC]ATQRLANFLVHSSNN(αMePhe)GPILPPTRVGSNTY-amide
SEQ ID NO: 16
K(γE-C18diacid)[CNTATC]ATQRLANFLVHSSNN(αMePhe)GPILPPTRVGSNTY-amide
SEQ ID NO: 17
K(γE-C18diacid)K[CNTATC]ATQRLA(Dab)FLVHSSNNFGPILPPTNVGSNTY-amide
SEQ ID NO: 18
K(γE-C18diacid)K[CNTATC]ATQRLANFLVHSS(Aib)NFGPILPPTHVGSNTY-amide
SEQ ID NO: 19
K(γE-C18diacid)[CNTATC]ATQRLANFLRHSS(Aib)NFGPILPPTEVGSNTY-amide
SEQ ID NO: 20
K(γE-C18diacid)[CNTATC]ATQRLANFLRHSS(Aib)NFGPILPPTNVGSNTY-amide
SEQ ID NO: 21
K(γE-γE-C18diacid)K[CNTATC]ATQRLANFLRHSS(Aib)NFGPILPPTNVGSNTY-amide
SEQ ID NO: 22
K(γE-γE-C18diacid)K[CNTATC]ATQRLANFLVHSS(Aib)NFGPILPPTRVGSNTY-amide
SEQ ID NO: 23
K(γE-C18diacid)K[CNTATC]ATQRLANFLVHSS(Aib)NFGPILPPT(Aib)VGSNTY-amide
SEQ ID NO: 24
K(γE-C18diacid)K[CNTATC]ATQRLANFL(Aib)HSSNNFGPILPPTNVGSNTY-amide
SEQ ID NO: 25
K(γE-C18diacid)K[CNTATC]ATQRLA(Dab)FL(Aib)HSSNNFGPILPPTEVGSNTY-amide
SEQ ID NO: 26
K[CNTATC]ATQRLANFLRHSSNN(αMePhe)GPILPPTEVGSNTY-amide
SEQ ID NO: 27
exemplary GLP-1(7-36) amide amino acid sequence
SEQ ID NO: 28
exemplary GLP-1(7-37) acid amino acid sequence
SEQ ID NO: 29
exemplary hGLP-1R amino acid sequence
SEQ ID NO: 30
exemplary hGCG amino acid sequence
SEQ ID NO: 31
exemplary hGCGR amino acid sequence
SEQ ID NO: 32
cotadutide amino acid sequence
SEQ ID NO: 33
H(Aib)QGTFTSDVSK(αMePhe)LDTKRARDFVQWLLE(Aib)G-acid
SEQ ID NO: 34
H(Aib)HGS(αMePhe)TSDVSK(αMePhe)LDSRAAKD(αMePhe)VQ(Aib)IANT-amide
SEQ ID NO: 35
H(Aib)QGTFTSDVSK(αMePhe)LDTK(O2Oc-O2Oc-γE-C18diacid)RARDFVQWLLE(Aib)G-acid
SEQ ID NO: 36
H(Aib)HGS(αMePhe)TSDVSK(αMePhe)LDSRAAK(ϵ-(O2Oc)-(O2Oc)-γE-
C18diacid)D(αMePhe)VQ(Aib)IANT-amide
SEQ ID NO: 37
H(Aib)QGTFTSDVSK(αMePhe)LDTK(O2Oc-O2Oc-γE-C20diacid)RARDFVQWLLE(Aib)G-acid
SEQ ID NO: 38
H(Aib)HGS(αMePhe)TSDVSK(αMePhe)LDSRAAK(E-(O2Oc)-(O2Oc)-γE-γE-C20diacid)D
(αMePhe)VQ(Aib)IANT-amide

### EXAMPLES

The invention is now described with reference to the Examples below. These are not limiting in terms of the scope of the invention, and a person skilled in the art would appreciate that suitable equivalents could be used within the scope of the present invention. Thus, the Examples may be considered component parts of the invention, and the individual aspects described therein may be considered as disclosed independently, or in any combination.

### Example 1: Lipidated Pramlintide Analogue Polypeptide and Lipidated-GLP-1/GCG Dual Agonist Polypeptide Preparation

Lipidated pramlintide analogue polypeptides were synthesized as described in WO 2022/129254. Briefly, lipidated pramlintide analogue peptides were synthesized as C-terminal carboxamides using rink amide MBHA resin (100-200 mesh). Polypeptides were prepared by automated synthesis using a Liberty Blue™ microwave solid phase peptide synthesizer (CEM Corporation, NC, USA) using the 9-fluorenylmethoxycarbonyl (Fmoc)/tert butyl (^{t}Bu) protocol.

Lipidated-GLP-1R/GCGR dual agonist polypeptides were synthesized as described in WO 2023/148399. Briefly, as described in WO 2022/129254. Briefly, lipidated-GLP-1R/GCGR dual agonist peptides were synthesized as C-terminal carboxamides or carboxylic acids using rink amide MBHA resin (100-200 mesh) or Wang resin (100-200 mesh). Polypeptides were prepared by automated synthesis using a PTI Prelude solid phase peptide synthesizer using the Fmoc/^{t}Bu protocol.

Manufacturer-supplied protocols were applied for coupling of amino acids in N,N-dimethylformamide (DMF) and deprotection of Fmoc protecting group using piperidine in DMF (20% v/v). Asparagine, cysteine, glutamine and histidine were incorporated as their sidechain triphenylmethyl, trityl (Trt) derivatives. Lysine was incorporated as the sidechain tert-butyloxycarbonyl (Boc) derivative. Serine, threonine and tyrosine were incorporated as sidechain tert-butyl (^{t}Bu) ethers, and aspartate and glutamate as their sidechain O'Bu esters. Arginine was incorporated as the sidechain 2,2,4,6,7-pentamethyldihydrobenzofuran-5-sulfonyl (Pbf) derivative. Other amino acids were incorporated with an appropriate sidechain protection.

Boc-Lys(Fmoc) was incorporated when a subsequent chemical modification of the N-terminal lysine side chain was required. Lys(Mmt) was incorporated when a subsequent chemical modification of the lysine side chain was required. Upon completion of the polypeptides chain elongation, Mmt side chain protection was removed by treatment of the resin with selective deprotection cocktail (1% trifluoroacetic acid (TFA), 5% TIPS in dichloromethane (DCM)) at 100 mL/mmol for 1 min, and repeated at least 10 times until Mmt group deprotection was completed. The reaction was quenched with 10% N,N-diisopropylethylamine (DIPEA)/NMP. Subsequent coupling of a albumin binding moiety, such as a lipid and linker, was performed manually using 1-[Bis(dimethylamino)methylene]-1H-1,2,3-triazolo[4,5-b]pyridinium 3-oxid hexafluorophosphate (HATU) as a coupling reagent in the presence of DIPEA.

Lipidated pramlintide analogue polypeptides were cleaved from the solid support by treatment with a mixture of TFA:TIS:EDT:thioanisole:water (90:2.5:2.5:2.5:2.5 v/v) for 4 h with agitation at room temperature. Lipidated-GLP-1R/GCGR dual agonist peptides were cleaved from the solid support by treatment with a mixture of TFA:TIS:water (92.5.5:2.5v/v) for 4 h with agitation at room temperature. Thereafter, the cleavage mixtures were filtered, concentrated in vacuo, precipitated and washed with diethyl ether and solids were isolated by centrifugation. The linear crude peptides were dried under a flow of nitrogen and dissolved in 20% MeCN/water (v/v) with 1% TFA (v/v) in water and filtered. The crude linear peptides were purified using a preparative RP-HPLC on a Varian SD-1 Prep Star binary pump system, monitoring by UV absorption at 210 nm using an Xbridge C18-A stationary phase (19.0×250 mm, 5 micron) column eluting a linear solvent gradient of 25-70% MeCN (0.1% TFA v/v) in water (0.1% TFA v/v) over 25 min.

The linear purified polypeptides were cyclised by treatment with iodine (1% w/v in methanol) for 10 min at room temperature and excess iodine was reduced by treatment with ascorbic acid (1% w/v in water). The cyclic crude polypeptides were re-purified as described above. The purified fractions were pooled, frozen and lyophilised.

LC/MS characterisation of purified polypeptides were performed on a Waters MassLynx 3100 platform using a XBridge C18 stationary phase (4.6×100 mm, 3 micron) eluting a linear binary gradient of 10-90% MeCN (0.1% TFA v/v) in water (0.1% TFA v/v) over 10 minutes at 1.5 mL/min at ambient temperature. Analytes were detected by both UV absorption at 210 nm and ionization using a Waters 3100 mass detector (ESI+ mode). Analytical RP-HPLC characterisation was performed on an Agilent 1260 Infinity system using an Agilent Polaris C8-A stationary phase (4.6×100 mm, 3 micron) eluting a linear binary gradient of 10-90% MeCN (0.1% TFA v/v) in water (0.1% TFA v/v) at 1.5 mL/min over 15 minutes at 40° C.

### Example 2: Combination Therapy with Selective Amylin Receptor (AMYR) Agonist Polypeptides and Lipidated-GLP-1/GCG Dual Agonist Polypeptides Drives Additional Body Weight Loss Compared with Monotherapy in DIO Rats

Study Design: Male diet-induced obese (DIO) Sprague-Dawley rats were fed a high fat diet (45% fat, D12451; Research Diets) for 34 weeks prior to study start. Additionally, age-matched lean rats were fed a control diet (10% fat, D12450K; Research Diets). DIO rats were then dosed (n=8/group) with Vehicle, GLP-1/GCG (5.4 nmol/kg), LAA (10 nmol/kg) or combination of a GLP-1/GCG dual agonist and a selective AMYR agonist (referred to as long-acting amylin analogue, LAA) subcutaneously (SC) once daily (QD) for 28 days. Average body weight for all DIO rats at start of study was 722.4+10.1 grams. Body composition was measured by EchoMRI on day-7 and day 26. In this experiment, the LAA was that of SEQ ID NO: 11, and the GLP-1/GCG dual-agonist was cotadutide.

Data reported as mean±standard error of the mean (SEM); data analysis performed using GraphPad Prism-statistical analysis performed using One-way ANOVA with Tukey ad hoc analysis. (*p<0.05, **p<0.01, ***0<0.001, ****p<0.0001)

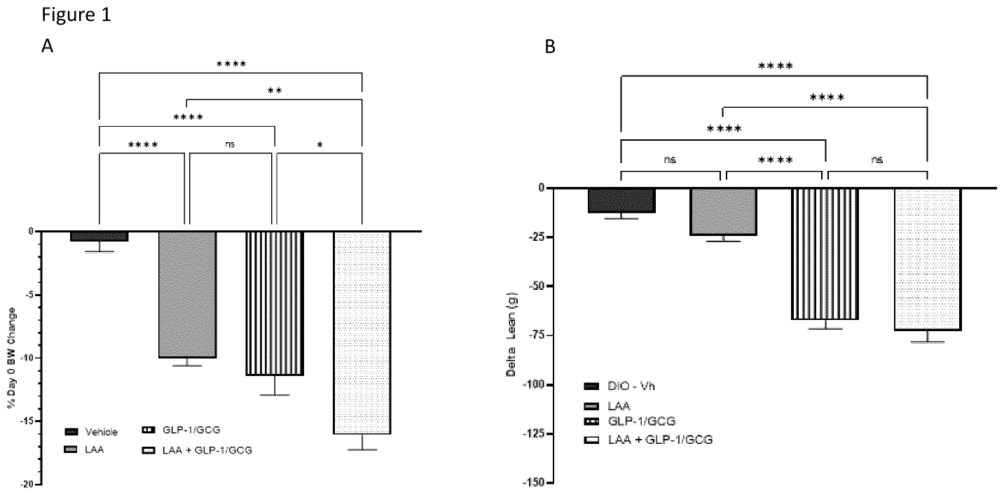

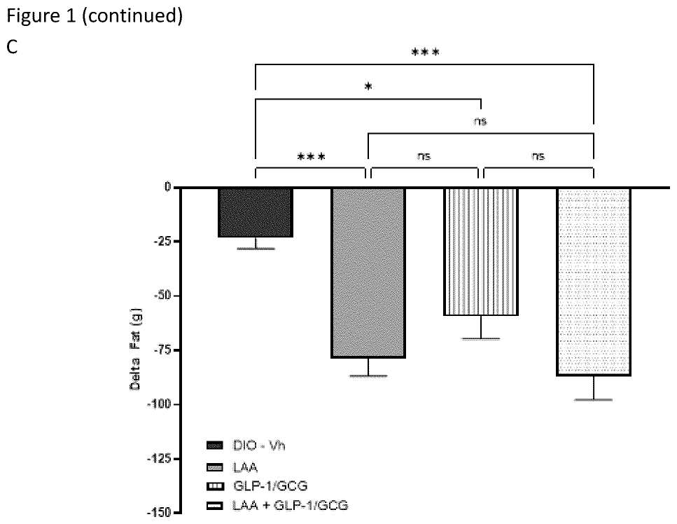

Study Data: DIO rats treated with LAA, GLP-1/GCG or a combination of both lost significantly more body weight than Vehicle treated rats. Additionally, animals treated with LAA+GLP1/GCG lost significantly more weight than either LAA or GLP-1/GCG mono treatments (FIG. 1A).

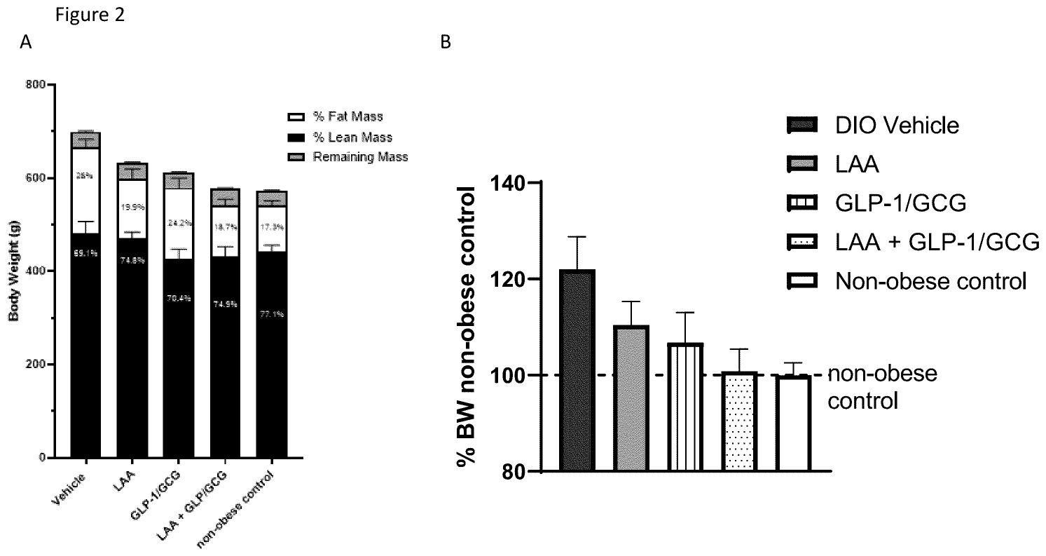

Further, as also shown with data from study day 26, animals treated with LAA+GLP1/GCG reached a body weight similar to non-obese control animals (FIG. 2B). Body composition as measured by EchoMRI (FIGS. 1B, 1C, 2A) shows that rats treated with LAA monotherapy lost a significant amount of fat mass compared to Vehicle treatment, with little effect on lean mass. Rats treated with GLP-1/GCG monotherapy lost both fat and lean mass compared to Vehicle treated animals. Rats receiving the combination of LAA and GLP-1/GCG lost both fat and lean mass. However, the effect on lean mass loss was not different from than of the GLP-1/GCG mono-treatment, suggesting the additional weight driven by combination with LAA is fat mass specific.

### Example 3: Confirmation that Combination Therapy with Selective Amylin Receptor (AMYR) Agonist Polypeptides and Lipidated-GLP-1/GCG Dual Agonist Polypeptides Drives Additional Body Weight Loss Compared with Monotherapy in DIO Rats

Study Design: Male DIO Sprague-Dawley rats were fed a high-fat diet (32.5% kcal fat, D12266B; Research Diets) for approximately 8 weeks prior to study start. Rats were dosed once daily SC for 28 days with Vehicle, GLP-1/GCG (1.5 nmol/kg or 5 nmol/kg), LAA (7.5 nmol/kg) or a combination of GLP-1/GCG and LAA (1.5 and 7.5 nmol/kg, or 5 nmol/kg and 7.5 nmol/kg, respectively) with an n=6/group. Body weight was monitored daily throughout the study. Data reported as mean±standard error of the mean (SEM); data analysis performed using GraphPad Prism-statistical analysis performed using One-way ANOVA with Tukey ad hoc analysis (*p<0.05, **p<0.01, ***0<0.001, ****p<0.0001). In this experiment, the LAA was that of SEQ ID NO: 11, and the GLP-1/GCG dual-agonist was cotadutide.

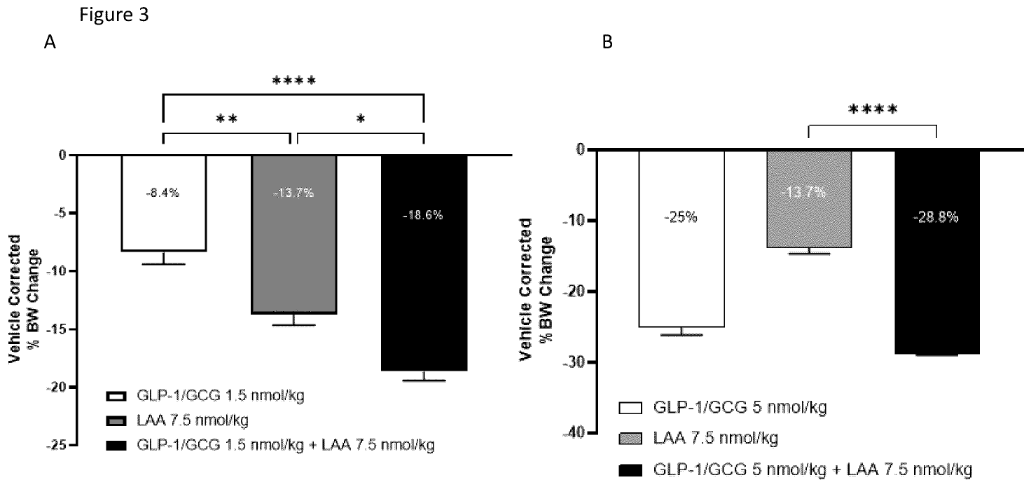

Data: At end of study (day 28) animals treated with GLP-1/GCG at 1.5 nmol/kg had a mean change in body weight of −8.4% and rats treated with LAA at 7.5 nmol/kg had a change of −13.7% when normalized to Vehicle. Rats that received both LAA and GLP-1/GCG treatment had a body weight change of −18.6% on day 28 when normalized to Vehicle (FIG. 3A). When rats were treated with GLP-1/GCG at 5 nmol/kg they had a mean body weight change of −25%, and when combined with LAA at 7.5 nmol/kg a mean body weight Vehicle corrected change of −28.8% was observed (FIG. 3B). Bliss independence modelling suggests this effect is additive for the LAA and GLP-1/GCG combination at 7.5 and 1.5 nmol/kg and trends towards synergy for the LAA and GLP-1/GCG combination at 7.5 and 5 nmol/kg, assuming that each compound is acting independently of one another and a maximum body-weight reduction of 30% in this preclinical model.

An additional study further confirms that the combination of a selective amylin receptor agonist and a GLP-1R/GCGR dual agonist enhances a reduction in body weight and food intake and delayed gastric emptying as compared to monotherapy in DIO rats.

Study design: DIO male pair-housed rats fed a 32.5% high-fat diet (D12266B) for 7 weeks reached a mean body weight (BW) of ˜595 g. Rats were dosed SC daily for 28 days with vehicle (Vh), GLP-1/GCG dual agonist (1.5 or 5 nmol/kg), LAA (2.5 or 7.5 nmol/kg), or combination (1.5+2.5 nmol/kg, 1.5+7.5 nmol/kg, 5+2.5 nmol/kg, or 5+7.5 nmol/kg, respectively). Food intake (FI) and BW were measured daily. In chow fed lean rats, gastric emptying (GE) was measured via X-ray fluoroscopic quantification following SC injections of Vh, GLP-1/GCG dual agonist at 1, 3, or 10 nmol/kg or LAA at 1, 3, or 10 nmol/kg. In a subsequent acute GE study, lean rats received a single dose of Vh, GLP-1/GCG dual agonist (2 nmol/kg), LAA (2 nmol/kg) or GLP-1/GCG dual agonist+LAA combination (2+2 nmol/kg). In this experiment, the LAA was that of SEQ ID NO: 11, and the GLP-1/GCG dual-agonist was cotadutide.

Study Data: Vh corrected BW change showed that GLP-1/GCG dual agonist monotherapy led to a mean decrease in BW gain of −8.4% and −25.0% at 1.5 and 5 nmol/kg, respectively. LAA monotherapy led to −9.4% and −13.7% BW gain at 2.5 or 7.5 nmol/kg, respectively. Combination of GLP-1/GCG dual agonist (1.5 nmol/kg) with LAA (2.5 nmol/kg) showed-14.2% BW gain, with −18.6% observed at 1.5+7.5 nmol/kg combination. GLP-1/GCG dual agonist (5 nmol/kg) with LAA (2.5 nmol/kg) combination led to −28.3% BW gain, and at 5+7.5 nmol/kg BW gain was-28.8%. The combination of GLP-1/GCG dual agonist with LAA demonstrated an additive effect on body weight according to the Bliss independence model.

Cumulative FI normalized to Vh was decreased in a dose-dependent manner with GLP-1/GCG dual agonist or LAA monotherapy, with a higher reduction in FI observed in combination treatment groups. Perirenal fat weights decreased in combination groups containing GLP-1/GCG dual agonist at 5 nmol/kg vs. Vh treatment. Liver weights decreased in all combination groups and GLP-1/GCG dual agonist monotherapy at 5 nmol/kg vs. Vh treatment. Liver lipids (%) were lower than Vh in all GLP-1/GCG dual agonist 5 nmol/kg treated groups.

In a GE study in lean rats, acute treatment with LAA or GLP-1/GCG dual agonist dose-dependently decreased GE represented as GE AUC as % of Vehicle (101%, 34% and 8% with LAA at 1, 3, and 10 nmol/kg and 85%, 49% and 24% with GLP-1/GCG dual agonist at 1, 3, and 10 nmol/kg respectively). In a subsequent GE study, combination of sub-maximal doses of 2 nmol/kg of LAA and GLP-1/GCG dual agonist showed a greater effect on GE compared to monotherapy arms.

Conclusion: DIO rats treated with a combination of LAA and GLP-1/GCG dual agonist displayed additive effect on BW gain vs. monotherapy. Additionally, peri-renal fat weight was decreased in the combination groups compared with GLP-1/GCG dual agonist dose of 5 nmol/kg, and decreased liver weights observed in all combination groups. Treatment with GLP-1/GCG dual agonist or LAA showed an expected decrease in GE in dose-dependent manner, and an additional effect on GE was observed with combination treatment at sub-maximal doses as compared to monotherapy, indicating a synergistic effect on gastric emptying in the combination group.

A further study confirms that the combination of a selective amylin receptor agonist and a GLP-1R agonist delays gastric emptying as compared to monotherapy in DIO rats.

Study Design: A repeat dose DIO rat gastric emptying (GE) study was performed with sub maximal doses of LAA (2 nmol/kg), GLP-1RA (2 nmol/kg) or their combination. X-ray fluoroscopic quantification was used to assess gastric emptying. In this experiment, the LAA was that of SEQ ID NO: 11, and the GLP-1R agonist was semaglutide.

Study Data: LAA monotherapy, unlike GLP-1RA monotherapy, maintained GE delay beyond 7 days of treatment. Greater body weight loss of the combination of the GLP1-RA and LAA correlated with an enhanced acute effect on GE.

### Example 4:3-Month Study of Selective Amylin Receptor (AMYR) Agonist Polypeptides and Lipidated-GLP-1/GCG Dual Agonist Polypeptides by Subcutaneous Administration in Wistar Han Rat

Study Design: Male (9-11 weeks of age) and female (10-11 weeks of age) Wistar Han rats were SC administered with Vehicle/Vehicle (group 1—for LAA of SEQ ID NO: 11 and GLP-1/GCG of SEQ ID NO: 35), LAA of SEQ ID NO: 11 and GLP-1/GCG of SEQ ID NO: 35 (group 2—500 and 5 μg/kg respectively), LAA of SEQ ID NO: 11 and GLP-1/GCG of SEQ ID NO: 35 (group 3—2000 and 5 μg/kg respectively), SEQ ID NO: 11 and Vehicle for SEQ ID NO: 35 (group 4—2000 μg/kg and 0 μg/kg respectively), or Vehicle for SEQ ID NO: 11 and SEQ ID NO: 35 (group 5—0 and 5 μg/kg respectively) daily for 91 days. Dosing for LAA of SEQ ID NO: 11 was dose escalated with group 2 receiving 250 μg/kg from day 1-5 and 500 μg/kg for duration of study, and groups 3 and 4 receiving 250 μg/kg SEQ ID NO: 11 from day 1-5, 500 μg/kg SEQ ID NO: 11 from days 7-13 and 2000 μg/kg for duration of study. There was a n=10/group per gender.

Study Data: Body weight comparison for rats (both male and female) at study day 92 were significantly lower in all groups receiving for LAA of SEQ ID NO: 11 or GLP-1/GCG of SEQ ID NO: 35 mono treatment or in combination (groups 2-5) than animals receiving only Vehicle administration. As shown in Table 1, data for male rats indicate a synergistic body-weight effect for the combination in Group 3 (according to Bliss independence model), while data for female rats indicate an additive effect for the combination in Group 3 (Bliss independence model), assuming that each compound is acting independently of one another and a maximum body-weight reduction of 30% in this preclinical model.

### Example 5: Combination Therapy with Selective Amylin Receptor (AMYR) Agonist Polypeptides and GLP-1 Agonist Polypeptides Drives Additional Body Weight Loss Compared with Monotherapy in DIO Rats

Study Design: Male DIO Sprague-Dawley rats were fed a high-fat diet (32.5% kcal fat, D12266B; Research Diets) for approximately 6 weeks prior to study start. Rats were dosed every other day (Q2D) SC for 19 days with Vehicle, GLP1-Fc (0.1 or 1 mg/kg), Fc-LAA (0.1 or 2 mg/kg) or a combination of GLP1-Fc and Fc-LAA (0.1+0.1 mg/kg, 0.1+2 mg/kg, 1+0.1 mg/kg or 1+2 mg/kg respectively) with an n=7/group. Body weight was monitored daily through-out study and shown below as % change of vehicle treatment. Data reported as mean±standard error of the mean (SEM). In this experiment, the LAA was an Fc-Amylin, and the GLP-1 agonist was dulaglutide (a GLP-1 agonist covalently linked to an Fc fragment of IgG4).

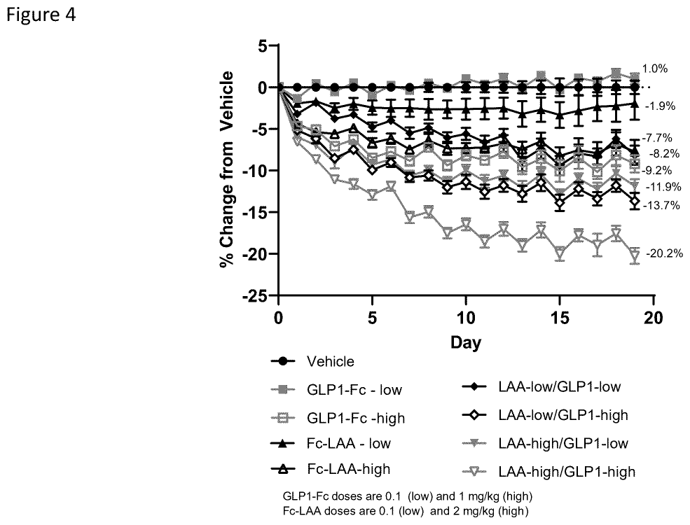

Study Data: As shown in FIG. 4, DIO rats treated with GLP1-Fc gained 1.0% or lost-9.2% body weight normalized to vehicle (0.1 and 1 mg/kg respectively). Rats treated with Fc-LAA mono-treatment lost weight in dose dependent manner with −1.9% or −7.7% (0.1 and 2 mg/kg respectively). In combination, there was further body weight loss observed. GLP1-Fc (0.1 mg/kg) and Fc-LAA (0.1 mg/kg) saw a change of −8.2%, GLP1-Fc (1 mg/kg) and Fc-LAA (0.1 mg/kg) observed a decrease of −13.7%, GLP-1-Fc (0.1 mg/kg) and Fc-LAA (2 mg/kg) treatment led to a decrease of −11.9%, and GLP-1-Fc (1 mg/kg) and Fc-LAA (2 mg/kg) had

TABLE 1
Effect on mean body weight for different treatments with the LAA of SEQ
ID NO: 11 or GLP-1/GCG of SEQ ID NO: 35 in mono- or combination therapy

| | Dose levels | | | | |
|---|---|---|---|---|---|
| | (g/kg/dose; | Mean Body | Mean Body | | Body Weight |
| | LAA of SEQ ID | Weight at | Weight at | Body Weight | Comparison, |
| | NO: 11 and GLP- | Day −1 | Day 92 | Gain (Day 92 | Day 92 vs. |
| Test Material | 1/GCG of SEQ ID | (grams, | (grams, | vs Day −1, | Control Group 1 |
| (Group No.) | NO: 35 | M/F) | M/F) | M/F) | Day 92 (M/F)^{a} |
| Group 1 | 0/0 | 282.8/208.5 | 405.3/250.3 | 143%/120% | 100%/100% |
| Group 2 | 500/5 | 276/201.3 | 279.8/197.1 | 100%/98% | 69%**/78%** |
| Group 3 | 2000/5 | 269.6/210.2 | 258.9/207 | 96%/98% | 64%**/83%** |
| Group 4 | 2000/0 | 268.6/209.8 | 319.2/222.1 | 119%/110% | 79%**/89%** |
| Group 5 | 0/5 | 274/201.6 | 325.5/225.4 | 119%/112% | 80%**/90%** |

^{a}Comparison of control group means (Group 1) to corresponding treated groups. Asterisks indicate statistically significant lower weights compared to control (**= p ≤ 0.01 at Day 92). Body weights were statistically significantly lower than control body weights beginning on Day 4 through Day 92 for males in Groups 2 to 5 and Group 2 females, beginning Day 18 through Day 92 for Groups 3 and 4 females, and beginning Day 77 through Day 92 for Group 5 females.

had an observed decrease of −20.2% body weight normalized to vehicle treatment. All GLP1-Fc and Fc-LAA combinations were predicted to be synergistic according to the Bliss independence model, assuming that each drug acts independently of the other, and a maximum body-weight reduction of 30% in this preclinical model.

### Example 6: Switching to Therapy with Selective Amylin Receptor (AMYR) Agonist Polypeptide Maintained Body Weight Loss and Prevented Body Weight Rebound after Discontinuation of GLP-1 Agonist Polypeptide Treatment in DIO Rats

Study Design: Male DIO Sprague-Dawley rats were fed a high-fat diet (32.5% kcal fat, D12266B; Research Diets) for approximately 8 weeks prior to study start. Rats were dosed SC for 15 days with Vehicle (n=16) or GLP-1RA (n=32) (5 nmol/kg) once-daily (QD). Beginning on day 16 rats initially treated with Vehicle either continued with Vehicle (n=8) or were switched to LAA (7.5 nmol/kg, n=8) administration. Rats initially receiving GLP-1RA treatment either continued with GLP-1RA, were switched to LAA (7.5 nmol/kg), were switched to Vehicle, or continued with GLP-1RA with addition of LAA treatment (7.5 nmol/kg) for duration of study (day 30), with n=8/group. Body weight was measured daily through-out study. Data reported as mean±standard error of the mean (SEM); data analysis performed using GraphPad Prism In this experiment, the LAA was that of SEQ ID NO: 11, and the GLP-1R agonist was semaglutide.

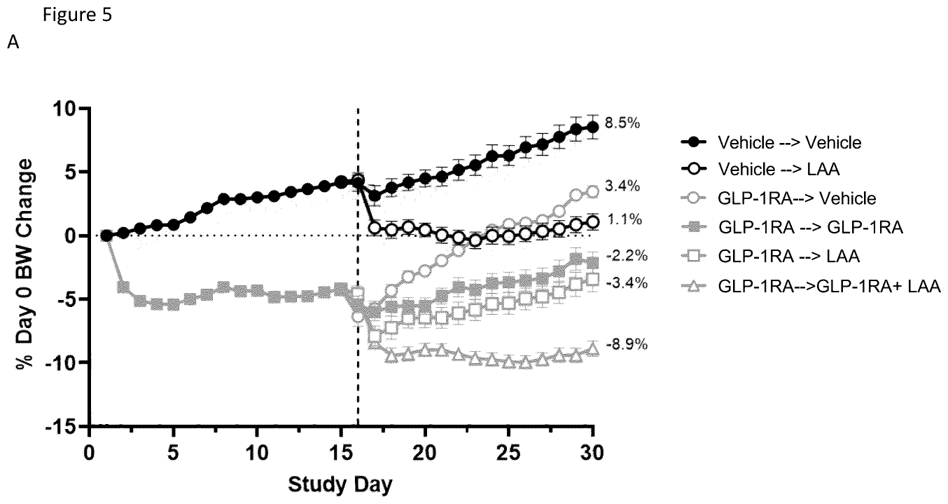

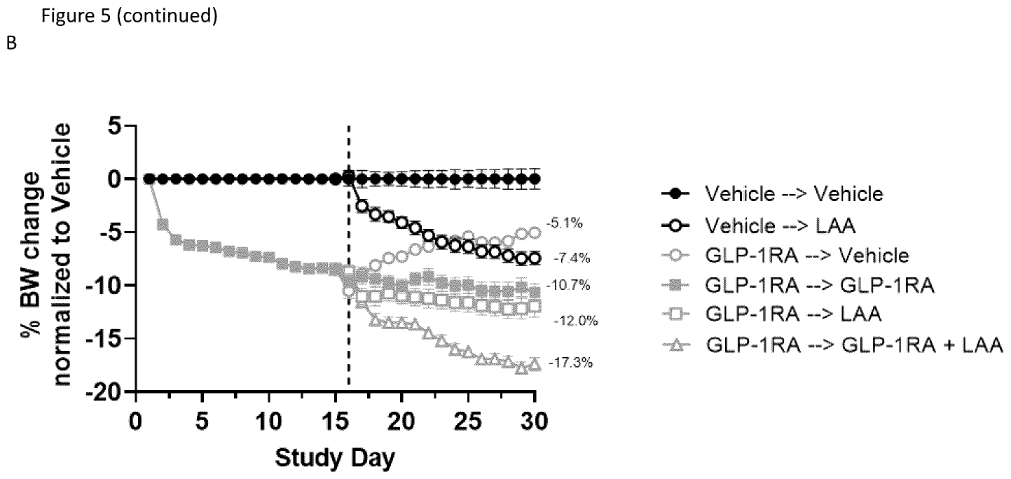

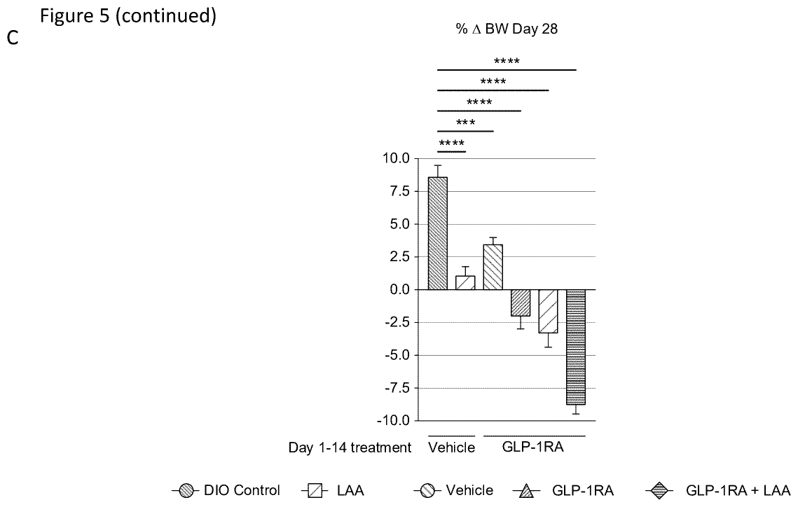

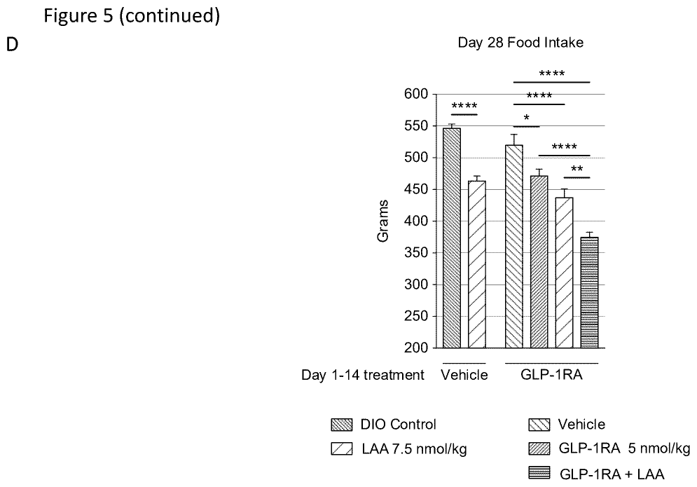

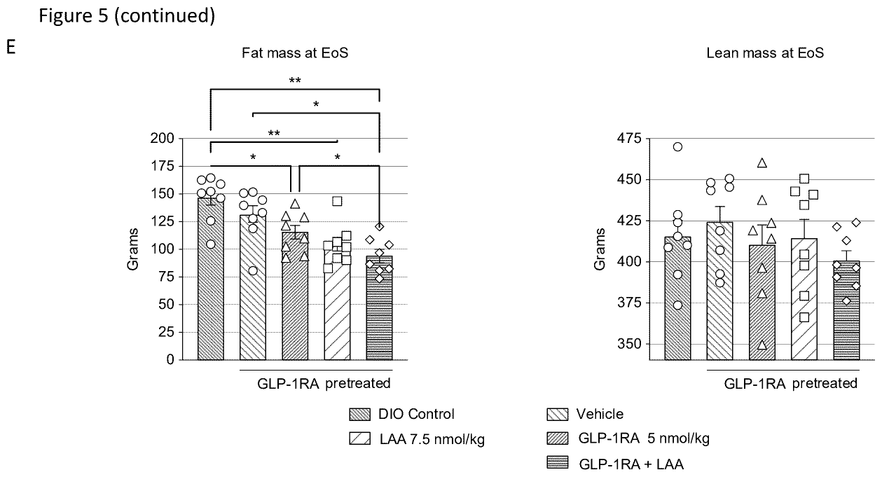

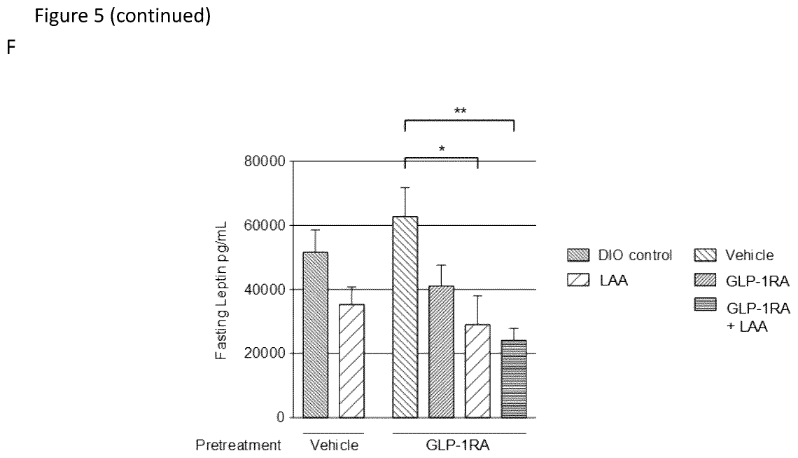

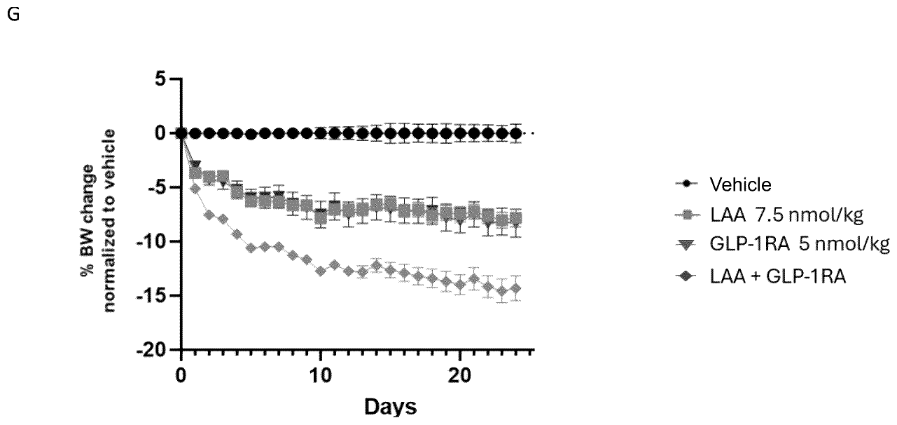

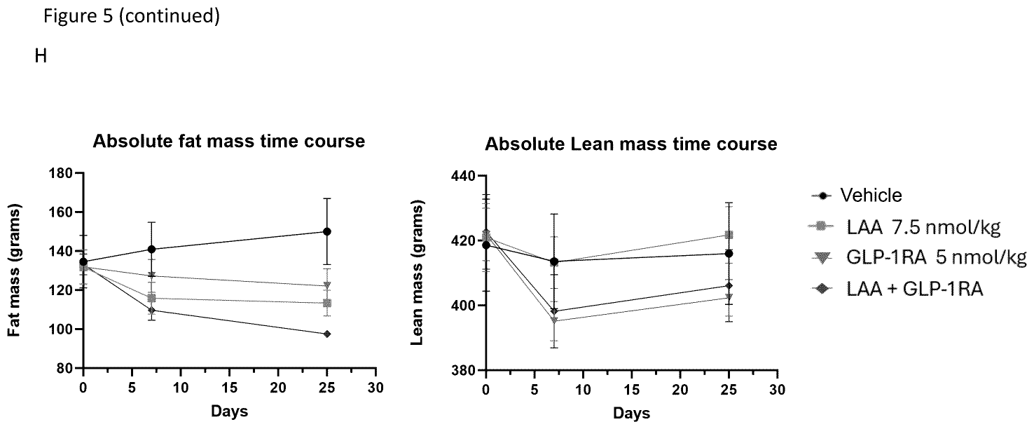

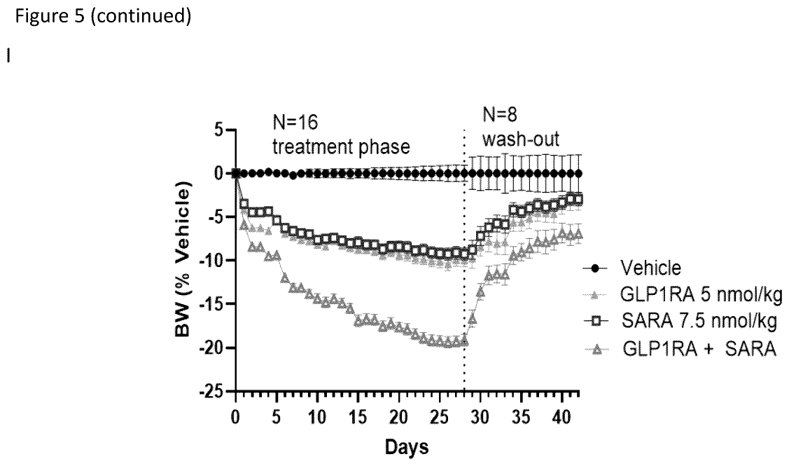

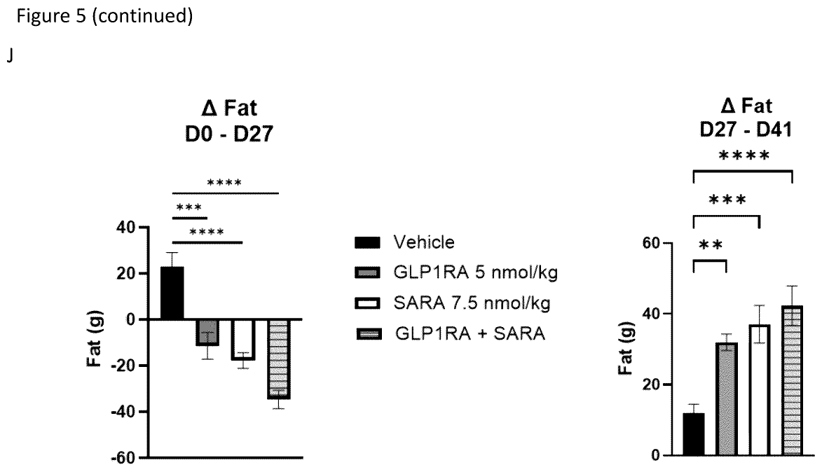

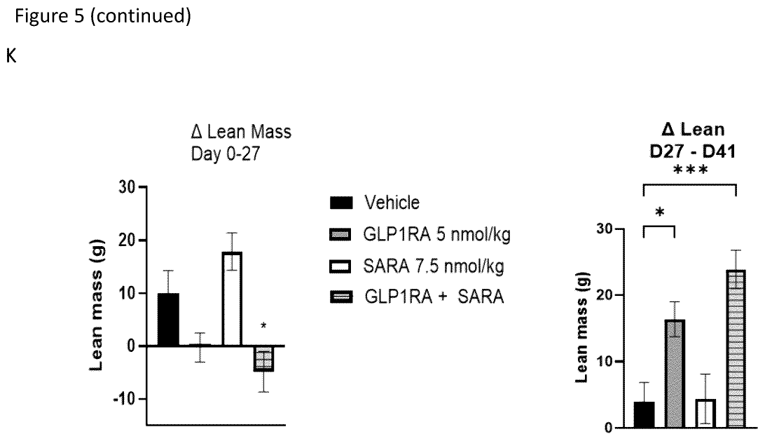

Study Data: As shown in FIG. 5A, DIO rats treated with a GLP-1RA lost weight following 15 days of treatment, mean of −4.2% of day 0 body weight, while animals treated with Vehicle gained an average of +4.2% of day 0 body weight. Following treatment switch, rats maintained on Vehicle treatment continued to gain weight (+8.5% of day 0 body weight) through end of study (day 30), whereas rats that were switched from Vehicle to LAA lost weight from day 16 to day 30, with an overall mean of +1.1% day 0 body weight. Rats that started with GLP-1RA treatment and continued to receive only GLP-1RA maintained body weight effect with an average BW loss of −2.2% at day 30; and animals that were switched from GLP-1RA to LAA treatment had a BW loss mean of −3.4%. Rats switched from GLP-1RA to Vehicle gained weight from day 16 to day 30 with a mean of +3.4% from their Day 0 body weight. Rats that started with GLP-1RA treatment and continued with GLP-1RA plus LAA administration lost more weight than mono-treatment groups with a mean of −8.9% weight loss at day 30 (as shown in FIG. 5C). When body weight was normalized to the vehicle treatment through duration of study (as shown in FIG. 5B), rats that were switched from VH to LAA had a −7.1% change in body weight. Animals treated with GLP-1RA for the duration of the study had a −10.7% change in body weight, while animals that were switched from GLP-1RA to LAA had a −12% change, animals switched from GLP-1RA to Vehicle weighed −5.1% less, and animals that remained on GLP-1RA plus LAA for second half of study had a −17.3% change in body weight when normalized to Vehicle treatment—this data suggests that with a lead in phase of GLP-1RA treatment, the addition of LAA may lead to further weight loss than mono-treatment. Bliss independence model indicates an additive effect (with a trend towards synergy) for combinations of LAA and GLP-1RA during Day 16-30, after pretreatment with GLP-1RA on Day 1-15, assuming that each compound is acting independently of one another and a maximum body-weight reduction of 30% in this preclinical model.

As shown in FIG. 5D, the greatest reduction in food intake at the end of the study was observed in the animals that maintained GLP-1RA therapy combined with the LAA adjunct therapy for the second half of the study. This reduction was significantly greater than in animals either continuing GLP-1 monotherapy throughout or switching from GLP-1 therapy to LAA therapy at day 15.

FIG. 5E represents the body composition data at the end of the study. Animals continuing GLP-1RA monotherapy throughout the study lost a small amount of fat mass as compared to control. In contrast, animals switching to LAA therapy at day 15 lost approximately 11 g more fat mass by the end of the study as compared to animals remaining on GLP-1RA monotherapy, and animals maintaining GLP-1RA therapy combined with the LAA adjunct therapy demonstrated almost 21 g reduction as compared to maintaining GLP-1RA therapy (see too Table 2). Total lean mass was similar in all groups, with a trend towards the animals switching to LAA therapy preserving the most lean mass of the groups continuing treatment to the end of the study.

TABLE 2
Fat mass data at the end of the study for DIO control
and treatment groups following initial GLP-1RA therapy

| | Fat | Δ from | | Δ from | Δ from |
|---|---|---|---|---|---|
| | (g) | Control | p-value | Vehicle | GLP-1RA |
| Control | 145.64 | — | — | — | — |
| Vehicle | 130.90 | 14.74 | 0.31 (ns) | — | — |
| GLP-1RA | 114.79 | 30.85 | 0.0187 | 16.11 | — |
| LAA | 103.70 | 41.95 | 0.0045 | 27.21 | 11.09 |
| Combination | 93.85 | 51.79 | 0.0047 | 37.05 | 20.94 |

FIG. 5F represents fasting leptin levels at the end of the study. Leptin levels were significantly reduced in animals switching to LAA monotherapy after an initial GLP-1RA therapy and animals maintaining GLP-1RA combined with LAA adjunct therapy, as compared to discontinuing therapy at day 15. Reduction of high levels of endogenous leptin in obese subjects is associated with restoration of leptin sensitivity.

The body weight loss and body composition effects from the combination of LAA with GLP-1RA was confirmed in a further study.

Study 2 Design: Male DIO Sprague-Dawley rats were injected QD SC with either vehicle, LAA 7.5 nmol/kg, GLP-1RA 5 nmol/kg, or both, for 4 weeks. Monotherapy doses were selected to achieve matching BW loss. BW and food intake were measured daily, and body composition was measured at baseline and end of study.

Study 2 Data: Rats treated with LAA or GLP-1RA showed similar BW loss when compared to vehicle, whereas the combined treatment resulted in greatest BW loss (see FIG. 5G). Vehicle-treated rats gained fat mass from baseline, whereas all treatment groups lost fat mass. Monotherapy with LAA led to a greater loss of fat mass than GLP-1RA monotherapy, while the combination group lost the most total fat mass. Lean mass loss in the combination group was not greater than the GLP-1RA monotherapy (see FIG. 5H).

Study 3 Design: Male DIO Sprague-Dawley rats (Charles River Laboratories) were fed a high-fat diet (32.5% kcal fat, D12266B; Research Diets) for approximately 8 weeks prior to study start, until they reached approximately 650 g where they were randomized into groups on body weight and body composition. Rats were dosed SC QD for 28 days with Vehicle (n=16), GLP-1RA at 5 nmol/kg (n=16), LAA (“SARA”) at 7.5 nmol/kg (n=16), or GLP1RA+LAA at 5+7.5 nmol/kg respectively (n=16). Following 28 days of treatment half the animals per group stopped treatment and were allowed to wash-out for 14 days (n=8/group), while the remaining animals per group were euthanized with blood/tissues collected. Body weight and food intake were measured daily, and body composition was measured before study start, on day 27 and then again on day 42. Data reported as mean±standard error of the mean (SEM); data analysis performed using GraphPad Prism. In this experiment, the LAA was that of SEQ ID NO: 11, and the GLP-1RA was semaglutide.

Study 3 Data: Treatment with LAA or GLP-1RA led to an inhibition of food intake of ˜−20%, p<0.0001 and −41%, p<0.0001 for the combination compared to vehicle (data not shown). Rats treated with LAA or GLP-1RA showed similar BW loss (−9.3% and −9.9%, respectively) when compared to vehicle (p<0.0001), whereas the combined treatment resulted in greatest BW loss of −19.1%, p<0.0001 (see FIG. 5I). Vehicle-treated rats gained +22.8 g of fat mass and +9.9 g of lean mass from baseline. Monotherapy with LAA led to a loss of fat mass of −17.7 g, p<0.0001, and monotherapy with GLP-1RA led to a loss of −11.4 g, p<0.001, while the combination group lost the most fat mass of −34.7 g, p<0.0001 (see FIG. 5J). LAA-treatment led to a preservation of lean mass, with slight reductions of lean mass demonstrated in both the GLP-1RA and combined treatment groups (see FIG. 5K). Cessation of treatment led to an increased in body weight and fat mass in all treatment groups, and an increase in lean mass in the GLP-1RA and combination groups.

Thus, treatment with a combination of LAA and GLP-1RA resulted in an increased suppression of food intake, more body weight loss, and more fat mass loss as compared to monotherapy.

### Example 7: Combination Therapy with Selective Amylin Receptor (AMYR) Agonist and a GLP-1/GIP Dual Agonist Polypeptide Resulted in Greater Body Weight Loss in DIO Rats Compared with Monotherapies

Study Design: Male rats were fed a high-fat diet (45% kcal fat-D12451; Research Diets) or chow control matched diet (10% kcal fat-D12450K; Research Diets) for 17-21 weeks prior to study start. DIO rats were dosed once daily SC for 28 days (n=8/group) with either Vehicle, LAA (10 nmol/kg), GLP/GIP dual agonist (2, 10 or 30 nmol/kg), or a combination of LAA (10 nmol/kg for all groups) and GLP/GIP dual agonist (2, 10 or 30 nmol/kg). Control rats were dosed SC with Vehicle. Body weight was monitored daily through-out study and body composition measured by EchoMRI at baseline (week-1) and end of study (week 4). In this experiment, the LAA was that of SEQ ID NO: 11, and the GLP-1/GCG dual-agonist was tirzepatide.

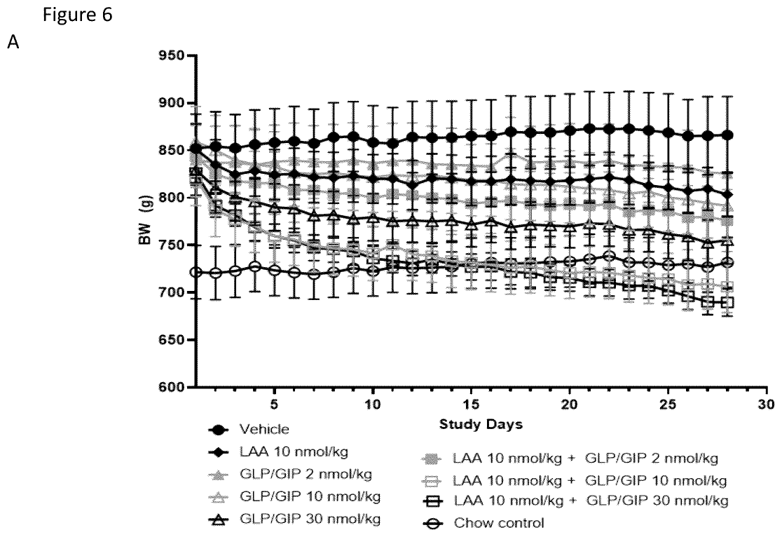

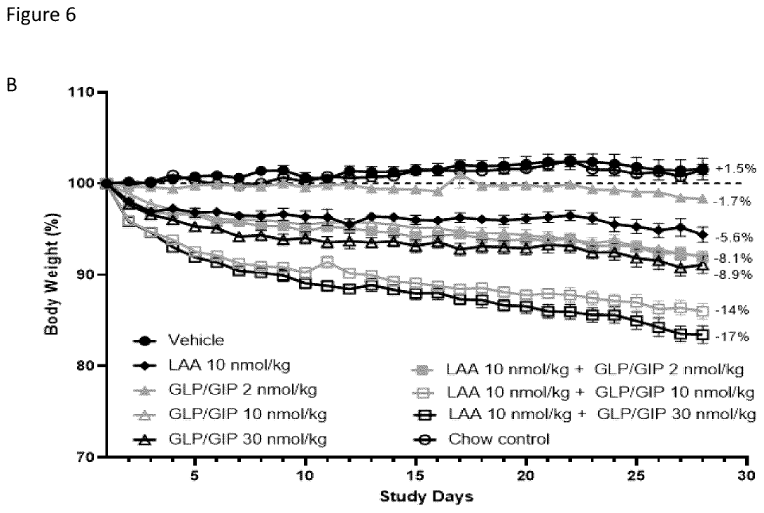

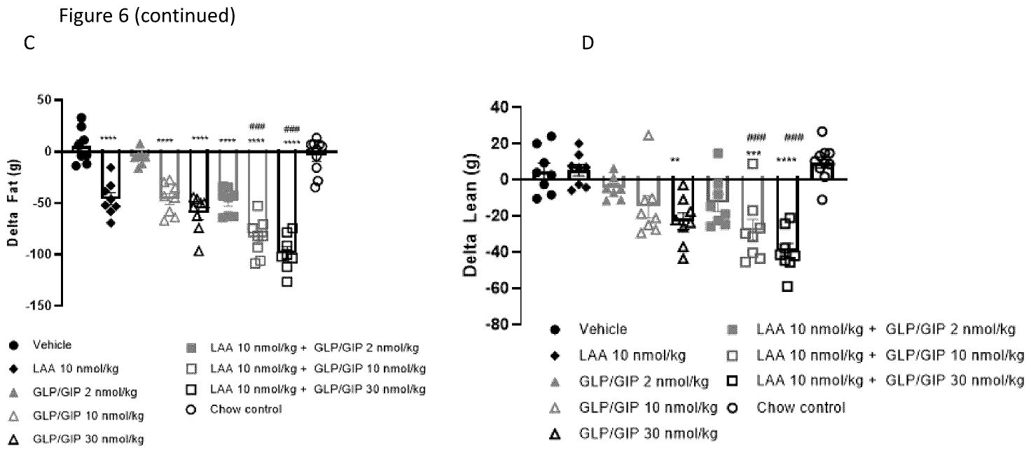

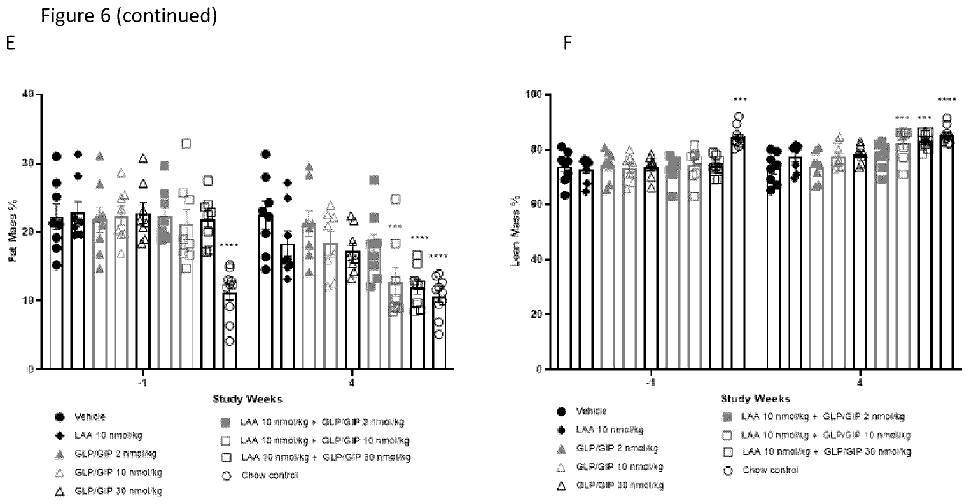

Study Data: As shown in FIGS. 6A and 6B, rats that were treated with Vehicle gained 1.5% body weight over duration of study (both control and HFD groups). Rats treated with LAA mono-treatment saw a body weight change of −5.6% on day 28, and rats treated with GLP/GIP mono-treatment lost weight in a dose-dependent manner at −1.7, −8.1 and −8.9% at 2, 10 and 30 nmol/kg respectively. Rats that were treated with LAA and GLP/GIP in combination had a further decrease in BW. LAA was fixed at 10 nmol/kg in all combination groups with GLP/GIP and a dose-dependent BW decrease of −8.1, −14 and −17% was observed when LAA combined with GLP/GIP at 2, 10 and 30 nmol/kg respectively. Bliss independence modelling suggests this is an additive effect with LAA and combination of GLP/GIP at 2 or 10 nmol/kg. LAA and GLP/GIP at 30 nmol/kg suggests a synergistic effect on body weight assuming a max BW reduction of 30% in this preclinical model.

Body composition measured at end of study showed a significant decrease in fat mass (g) (FIG. 6C) with LAA mono-treatment, and a dose-dependent decrease in fat mass in GLP/GIP mono-treatment groups. The combination of LAA and GLP/GIP saw a further decrease in fat mass in a dose-dependent manner. As shown in FIG. 6D, LAA mono-treatment in rats saw no effect on lean mass (g) change, where GLP/GIP mono-treatment had a dose-dependent decrease of lean mass-no effect at 2 nmol/kg but a significant reduction vs. vehicle at 30 nmol/kg. When LAA and GLP/GIP were treated in combination there was a significant reduction in lean mass in groups receiving GLP/GIP at either 10 or 30 nmol/kg. When body composition was normalized to body weight, rats treated with LAA and GLP/GIP at either 10 or 30 nmol/kg had a significant decrease of % fat mass compared to Vehicle, and similar % fat mass to chow control animals (FIG. 6E). Lean mass normalized to body weight showed a significant increase in % lean mass in animals receiving LAA in combination with GLP/GIP at 10 or 30 nmol/kg (FIG. 6F).

### Example 8: Combination Therapy with Selective Amylin Receptor (AMYR) Agonist Resulted in No Worsening of Aversion to Saccharin a GLP-1 R Agonist Polypeptide Unlike Combination Therapy with a GLP-1 Agonist Polypeptide and a Dual Amylin and Calcitonin Receptor Agonist (DACRA)

Study 1 Design: Male Wistar Han rats (9 weeks old) on a chow diet had overnight water restriction on day −1. On study day 1 they were given saccharin water for 4 hours and during this time were dosed once SC with either Vehicle, GLP1RA (2.4 nmol/kg), a dual amylin/calcitonin receptor agonist (DACRA; 10 nmol/kg), LAA (30 nmol/kg), GLP1RA and DACRA (2.4 nmol/kg and 10 nmol/kg respectively) or GLP1RA and LAA (2.4 nmol/kg and 30 nmol/kg respectively). After a 4-hour exposure to saccharin, it was removed and rats had access to water as usual. A preference test was performed 72 hours after compound administration. During this time, rats are given a choice of both saccharin and water, and the intake of both are measured over a 24-hour period. Rats generally prefer the sweet saccharin water, and if they associate the saccharin water with feeling malaise or nausea they will not drink as much of the saccharin and prefer water. The saccharin preference is calculated a percent of saccharin intake (g)/total intake (saccharin and water). Data reported as mean±standard error of the mean (SEM); data analysis performed using GraphPad Prism—statistical analysis performed using One-way ANOVA with Tukey ad hoc analysis. (*p<0.05, **p<0.01, ***0<0.001, ****p<0.0001 vs. Vehicle). In this experiment, the LAA was that of SEQ ID NO: 11, the GLP-1 agonist was semaglutide and the DACRA was cagrilintide.

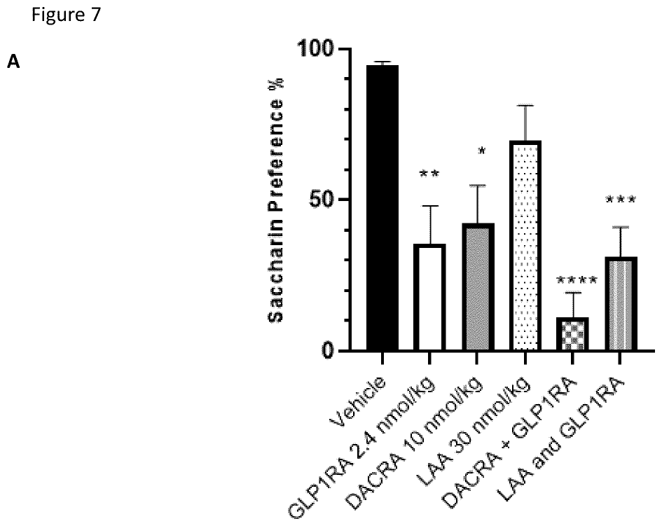

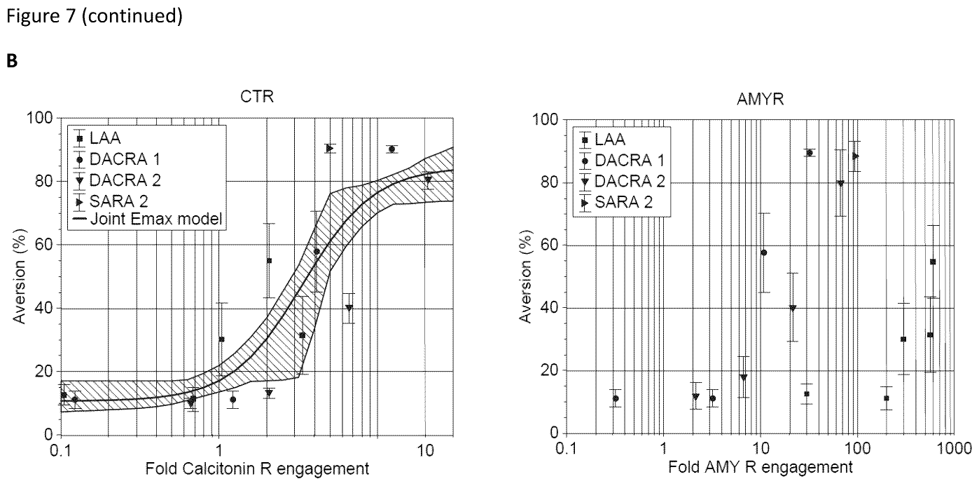

Study 1 Data: As shown in FIG. 7A, rats treated with vehicle had a saccharin preference of >90%, while animals treated with GLP-1RA mono-treatment (˜35%) or DACRA (˜42%) mono-treatment had a significantly lower saccharin preference. LAA mono-treated rats did not have a significantly lower saccharin preference (˜70%), although not as high as Vehicle treated rats—this suggests the LAA mono-treatment at 30 nmol/kg is not as aversive as observed with mono therapy with GLP1R agonists or DACRA compounds. Rats treated with GLP1RA and DACRA combination had a lower saccharin preference (˜11%) than Vehicle and tending to be lower than the monotreatment groups, whereas the GLP1RA and LAA combination group had a saccharin preference similar (˜31%) to GLP1RA monotreatment suggesting addition of LAA did not worsen the aversion of GLP1RA alone. For each compound, the dose level was selected to not have a maximal effect on aversion, in order to maintain a window to a potential added effect with the combination. Additionally, the LAA molecule exhibits ˜28 fold more amylin receptor engagement than the DACRA molecule, and the DACRA exhibits ˜2-3 fold more calcitonin engagement than LAA-suggesting the aversion to saccharin water with the DACRA could be mediated via calcitonin receptor engagement.

Study 2: Indeed, a further aversion study in lean rats comprising two selective amylin receptor agonists (SARA) and two DACRAs demonstrates that saccharine aversion in lean rats correlates to calcitonin receptor engagement but not to amylin receptor engagement (see FIG. 7B). Target engagement was estimated in the following way for each compound. The maximum concentration (Cmax) after subcutaneous injection was measured in separate rat pharmacokinetic studies and was dose-adjusted to the tested dose levels when appropriate, assuming dose linearity. Plasma protein binding was determined in vitro using 3B Pharmaceutical's Escalate Equilibrium Shift Assay or an in-house Surface Plasmon Resonance (SPR) assay. Peptide in vitro potency was determined via HTRF CAMP accumulation assay using HEK293 cells recombinantly expressing the rat calcitonin receptor or rat amylin receptor (calcitonin receptor plus RAMP3) in saline buffer containing 0.1% bovine serum albumin. The in vitro potencies (IC50s) for amylin and calcitonin were adjusted to unbound values by taking into account the albumin concentration in the assay and fraction unbound data, according to an established method (Wan H and Bergström F (2007), High Throughput Screening of Drug-Protein Binding in Drug Discovery, Journal of Liquid Chromatography & Related Technologies, 30:5, 681-700; doi: 10.1080/10826070701190989). Finally, the target engagement was calculated as the unbound Cmax divided by the unbound IC50, both for amylin and calcitonin. The Emax model was defined as aversion=E0+Emax×(ENG\hat{ }h/(ENG\hat{ }h+ED50\hat{ }h)), where E0 is the baseline aversion, Emax is the maximum induced aversion, ENG is the receptor engagement, and h is the slope (Hill) parameter. Parameter estimation was performed according to a maximum likelihood approach with an additive error model, incorporating data from all compounds simultaneously. Uncertainty of parameter estimates was determined by bootstrapping, sampling single measurements randomly with replacement within each experiment (N=500). Numeric analyses were performed in MATLAB (R2023b; The MathWorks, Natick, MA). The aversion data are given as mean and standard error (N=9-11 per group). In this experiment, the LAA/SARA1 was that of SEQ ID NO: 11, SARA2 was eloralintide, DACRA1 was cagrilintide, and DACRA2 was petrelintide.

TABLE 3
Relative potency ratios

Relative potency ratio
(AMY3R/CTR), ^{ab}

| Molecule | | Rat | Human |
|---|---|---|---|
| hAmylin | SARA | 0.9 | 1.8 |
| Pramlintide | | 1.0 | 1.0 |
| LAA/SARA 1 | SARA | 1.6 | 0.9 |
| SARA 2 | SARA | 0.14 | 0.24 |
| DACRA 1 | DACRA | 0.01 | 0.08 |
| DACRA 2 | DACRA | 0.03 | 0.05 |

^{a} Assays in the presence of 0.1% bovine serum albumin, n ≥ 5.
^{b} Relative potency ratio = (amylin assay potency relative to pramlintide 100%)/(calcitonin assay potency relative to pramlintide 100%).
AMY, amylin;
CTR, calcitonin, all except hAmylin and Pramlintide are lipidated peptides

Overall, this supports the use of selective amylin receptor (AMYR) agonists in combination with GLR-1R agonists according to the invention.

### Example 9: Selective Amylin Receptor (AMYR) Agonist Monotherapy Drives Fat-Mass Specific Body Weight Loss and Preserves Lean-Mass as Compared to a Dual Amylin and Calcitonin Receptor Agonist (DACRA) Monotherapy in DIO Rats

Study 1 Design: High-fat diet (DIO) fed rats were used to profile LAA or DACRA monotherapy to evaluate food intake, body weight-lowering and body composition effects in a 4-week study. In this experiment, the LAA was that of SEQ ID NO: 11 and the DACRA was cagrilintide.

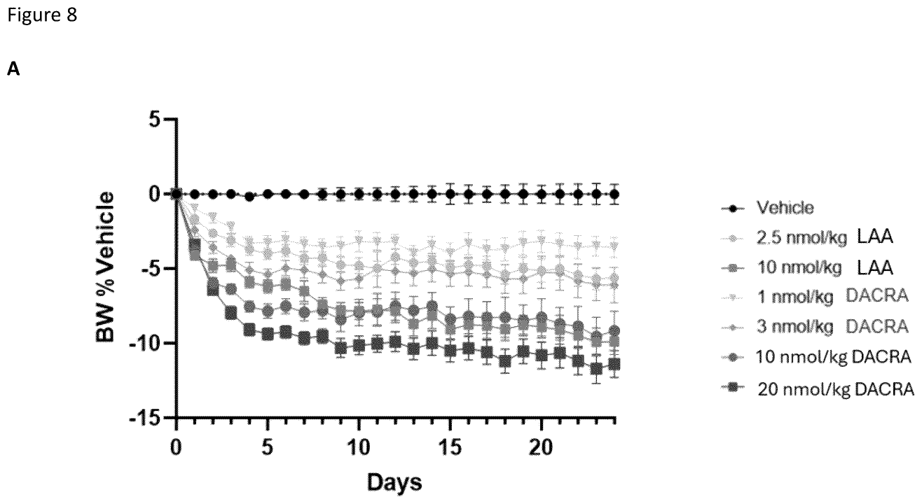

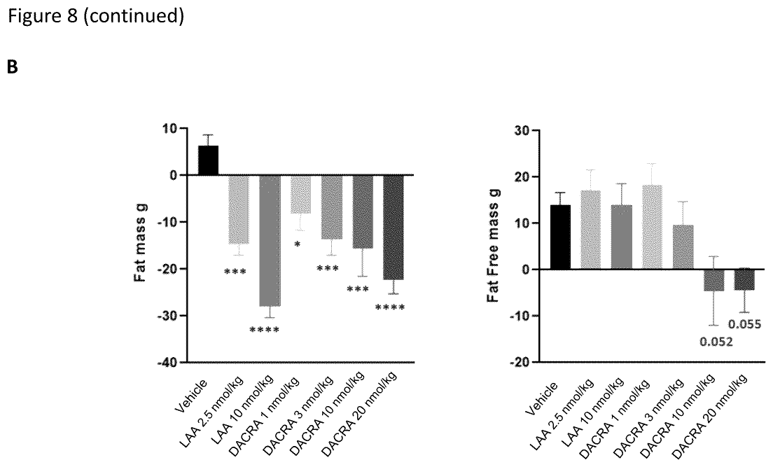

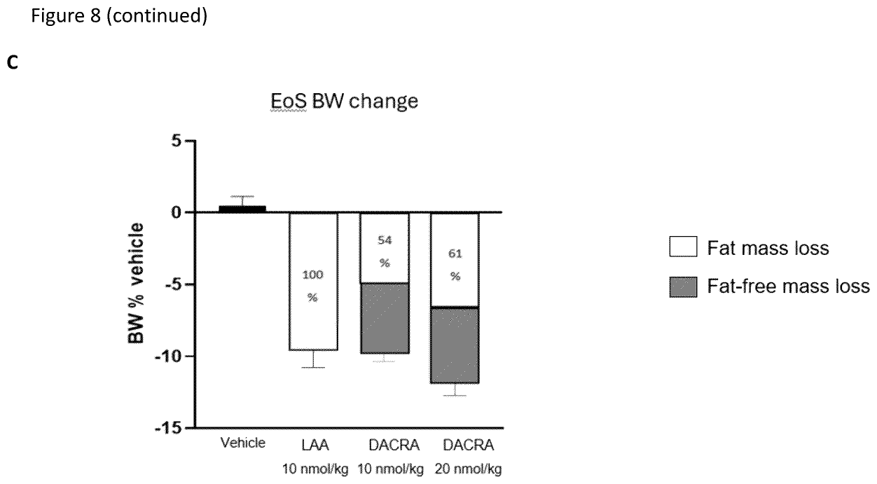

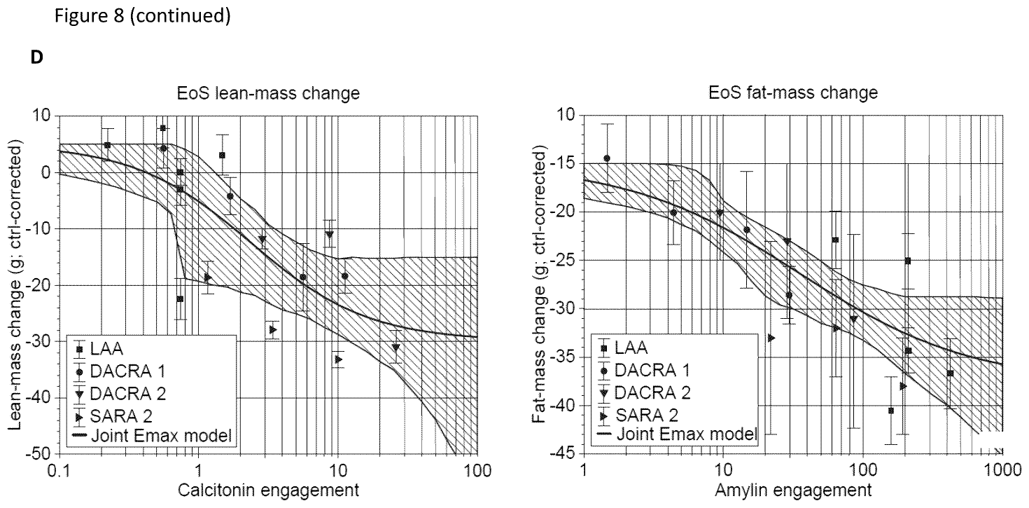

Study 1 Data: LAA (2.5 or 10 nmol/kg) or DACRA (1, 3, 10, or 20 nmol/kg) over a 4-week study in DIO rats resulted in dose-dependent reduction in body weight compared to vehicle treated rats. BW reduction was −5.2±0.6 and −9.7±0.7% for LAA (2.5 or 10 nmol/kg), and −3±0.6, −5.3±1.1, −8.8±1.4 and −11.6±0.9% for DACRA (1, 3, 10, or 20 nmol/kg), respectively (see FIG. 8A). A reduction of cumulative food intake of −14.3±1.2 and −20.9±0.7% of vehicle for LAA (2.5 or 10 nmol/kg), and −7.1±2.1, −16±2.5, −23.9±3.5 and −23.7±1.1% of vehicle for DACRA (1, 3, 10, or 20 nmol/kg), respectively, was observed. Body-composition measurements showed that LAA, unlike DACRA, promoted fat-mass-specific body weight loss while preserving lean-mass. The greatest fat-mass loss observed for LAA 10 nmol/kg was −28.0±2.3 g, which was nearly double that observed for matched body weight loss DACRA 10 nmol/kg of −15.5±6.0 g fat. Lean mass was preserved in the LAA groups, unlike in the DACRA groups (see FIG. 8B). Overall, LAA resulted in more fat mass loss and preservation of lean mass as compared to DACRA at matched body weight loss levels. In particular, 100% of body weight loss was fat-mass specific in the LAA 10 nmol/kg group, as compared to 54% and 61% for DACRA 10 and 20 nmol/kg groups, respectively (see FIG. 8C).

Study 2 Design: Target engagement was estimated in the following way for each compound. The average steady-state concentration (Cavg) after repeated subcutaneous injection was estimated in rats based on measured rat clearance in separate rat pharmacokinetic studies, dose level, and dosing interval. Plasma protein binding was determined in vitro using 3B Pharmaceutical's Escalate Equilibrium Shift Assay or an in-house Surface Plasmon Resonance (SPR) assay. Peptide in vitro potency was determined via HTRF CAMP accumulation assay using HEK293 cells recombinantly expressing the rat calcitonin receptor or rat amylin receptor (calcitonin receptor plus RAMP3) in saline buffer containing 0.1% bovine serum albumin. The in vitro potencies (IC50s) for amylin and calcitonin were adjusted to unbound values by taking into account the albumin concentration in the assay and fraction unbound data, according to an established method (Wan H and Bergström F (2007), High Throughput Screening of Drug-Protein Binding in Drug Discovery, Journal of Liquid Chromatography & Related Technologies, 30:5, 681-700; doi: 10.1080/10826070701190989). Finally, the target engagement was calculated as the unbound Cavg divided by the unbound IC50, both for amylin and calcitonin. The Emax model was defined as Lean_mass=E0−Emax×(ENG\hat{ }h/(ENG\hat{ }h+ED50\hat{ }h)), where E0 is the baseline mass, Emax is the maximum induced mass change, ENG is the receptor engagement, and h is the slope (Hill) parameter. Parameter estimation was performed according to a maximum likelihood approach with an additive error model. Uncertainty of parameter estimates was determined by bootstrapping, sampling single measurements randomly with replacement within each experiment (N=500). Numeric analyses were performed in MATLAB (R2023b; The MathWorks, Natick, MA). The lean and fat mass changes are given as mean and standard error (N=8 per group).

Study 2 Data: Lean-mass loss correlated with the degree of calcitonin receptor engagement for DACRA, while fat-mass loss correlated with the degree of amylin receptor engagement (see FIG. 8D). This further supports the use of selective amylin receptor (AMYR) agonists according to the invention.

### Example 10: A Phase II Randomised, Double-blind, Placebo-controlled Study to Evaluate the Efficacy, Safety and Tolerability of a Selective Amylin Receptor (AMYR) Agonist in Participants Living with Overweight or Obesity with Type 2 Diabetes Who Are on a Stable Dose of GLP-1 Receptor Agonist

As per Example 6 above, preclinical studies demonstrated that when Male DIO Sprague-Dawley rats receiving a stable dose of a GLP-1 RA (semaglutide) had a LAA (SEQ ID NO: 11) added to their treatment regimen, these rats lost more weight than rats continuing with mono-treatment with the GLP-1 RA, or switched to mono-treatment with the LLA. In particular, there was a trend towards synergy when the LAA was added to the GLP-1 RA treatment.

Following the pre-clinical studies, a Phase II, randomised, parallel-group, double-blind, placebo-controlled, multi-centre study was designed to assess the efficacy, safety, and tolerability of a LAA compared with placebo, given once weekly as a subcutaneous (SC) injection, in adults living with overweight or obesity and type 2 diabetes who are on a stable dose of GLP-1 RA. In this study, the LAA is that of SEQ ID NO: 11.

The study was designed to be conducted at approximately 15 study sites in the USA. The study was designed for human male and female participants between 18 to 75 years of age (inclusive) at the time of screening, with a BMI≥27 kg/m² and clinical diagnosis of type 2 diabetes who are on a stable therapeutic dose of GLP-1 RA for 6 months, inclusive, at screening.

An eligibility screening protocol was designed. The screening period is up to 28 days prior to randomisation. The screening protocol was designed to include eligible consenting participants according to: (i) age (18 to 75 years of age, inclusive, at the time of signing the informed consent), disease characteristics (diagnosed with type 2 diabetes≥180 days before screening and a HbA1c value at screening of ≥7.0% (53 mmol/mol) and ≤10% (86 mmol/mol) managed with diet and exercise alone or with a stable dose of metformin and/or SGLT2 inhibitors and/or DDP4 inhibitors, where no dose change has occurred for at least 3 months prior to randomisation); (iii) on a stable maintenance dose of an injectable GLP-1 RA for 6 months at the time of screening; and (iv) at Screening, have a BMI≥27 kg/m². Exclusion criteria included (i) having received treatment with prescription or non-prescription medication for weight loss within the last 3 months prior to screening (other than a GLP-1 RA); (ii) self-reported weight change of >5% in the 3 months prior to screening; (iii) diabetes mellitus that is not clearly type 2 diabetes: i.e., type 1 diabetes, pancreatogenic diabetes including cystic fibrosis-related diabetes, posttransplant diabetes mellitus, monogenic diabetes (remote history of gestational diabetes is allowed); (iv) use of insulin therapy for type 2 diabetes mellitus; (v) previous or planned (within study period) bariatric surgery or fitting of a weight loss device (e.g., gastric balloon or duodenal barrier); (vi) significant hepatobiliary disease (except for non-alcoholic steatohepatitis or non-alcoholic fatty liver disease without portal hypertension or cirrhosis); (vii) serious cardiovascular conditions or cardiovascular risks that according to the investigator make the participant unsuitable to participate in the study; and (viii) impaired renal function defined as eGFR≤45 mL/minute/1.73 m² at screening.

Participants are screened/enrolled in the study with approximately 64 eligible participants randomised to receive study intervention. Eligible participants are randomised on Study Day 1 in a 1:1 ratio to receive either LAA or matching placebo with respect to titration schedule and dose levels, in addition to their GLP-1 RA stable therapeutic regimen. The investigator has the discretion to adjust the dosing schedule of LAA/placebo by maintaining or decreasing the dose based on the participant's intolerance to the designated dose. Approximately 32 participants will be randomised to each treatment arm as follows.

TABLE 2
Phase II treatment schedule

| Treatment arm | Intervention |
|---|---|
| LAA | Weekly SC injection |
| Placebo | Weekly SC injection |
| | of matching placebo |

Both treatment arms are in addition to (on top of) a patient's existing GLP-1 RA stable therapeutic regimen.

LAA or placebo will be administered once weekly via SC injections. Eligible participants will attend up to 26 visits during the treatment period, one end of treatment visit and one additional visit in the follow-up period.

In more detail, following the screening period, a 26-week Treatment Period commences during which participants receive study intervention once weekly from Study Week 0 until Study Week 25. Each once-weekly treatment comprises an SC injection. The visit frequency will be weekly for those who will receive the injections at clinic. Alternatively, visits may be less frequent for participants who have the option of administering subsequent doses of study intervention at home (home-dosing). After the end of the treatment period, there is a follow-up period of 7 weeks post last dose, with a final visit at Study Week 32.

### Primary Objectives

The co-primary objectives of this study are:

| Objectives | End-points |
|---|---|
| To determine whether treatment | Percent change in body |
| with LAA is superior to placebo | weight from baseline |
| for weight loss at Study Week 26 | at Study Week 26 |
| To assess the effect of LAA | Weight loss ≥5% |
| versus placebo on the proportion | from baseline |
| of participants with weight loss ≥5% | at Study Week 26 |
| from baseline at Study Week 26 | |

In particular, these primary objectives are in the context of determining whether treatment with LAA is superior to placebo for weight loss in participants with BMI≥27 kg/m² and type 2 diabetes, who are on a stable treatment dose of GLP-1 RA, for type 2 diabetes, after 26 weeks of treatment.

### Secondary Objectives

The secondary objectives of this study are:

| Objectives | End-points |
|---|---|
| To assess the effect of LAA | Weight loss ≥10% |
| vs placebo on the proportion | from baseline |
| of participants with weight | at Study Week 26 |
| loss ≥10% from baseline | |
| at Study Week 26 | |
| To determine whether LAA | Absolute change in body |
| is superior to placebo for | weight (kg) from baseline |
| absolute weight reduction | at Study Week 26 |
| (kg) at Study Week 26 | |
| To assess the effect of LAA | Change in HbA1c and fasting |
| vs placebo on glycaemic | serum glucose from baseline |
| control at Study Week 26 | at Study Week 26 |
| To characterise the PK of LAA | LAA plasma concentrations |

In particular, these secondary objectives are in the context of determining whether treatment with LAA is superior to placebo for weight loss in participants with BMI≥27 kg/m² and type 2 diabetes, who are on a stable treatment dose of GLP-1 RA, for type 2 diabetes, after 26 weeks of treatment.

1. A method for treating and/or preventing a disease or disorder in a subject, the method comprising administering to the subject (a) an amylin receptor (AMYR) agonist, wherein the AMYR agonist has selectivity to AMYR as compared to a calcitonin receptor (CTR); and (b) a GLP-1 receptor (GLP-1R) agonist.

2. (canceled)

3. The method according to claim 1, wherein the method further comprises administering to the subject a glucagon receptor (GCGR) agonist.

4. The method according to claim 1, wherein the AMYR agonist, GLP-1R agonist, and/or GCGR agonist is a polypeptide, small molecule drug, antibody, antibody-drug conjugate, or aptamer; or a pharmaceutically acceptable salt thereof.

5-7. (canceled)

8. The method according to claim 1, wherein the AMYR agonist has at least a 2-fold, at least 3-fold, at least 4-fold, at least 5-fold, at least 6-fold, at least 7-fold, at least 8-fold, at least 9-fold, at least 10-fold, at least 12-fold, at least 15-fold, at least 17-fold, at least 20-fold, or at least 25-fold selectivity to AMYR as compared to CTR.

9-10. (canceled)

11. The method according to claim 1, wherein the AMYR agonist is an AMYR agonist polypeptide, or pharmaceutically acceptable salt thereof, which comprises an amino acid sequence having at least 90% identity to pramlintide (KCNTATCATQRLANFLVHSSNNFGPILPPTNVGSNTY-amide, SEQ ID NO: 5).

12. The method according to claim 11, wherein the AMYR agonist polypeptide is lipidated, wherein the lipid is selected from the group consisting of C12diacid, C14diacid, C16diacid, C17diacid, C18diacid, C19diacid or C20diacid.

13. The method according to claim 12, wherein the lipid is attached to an amino acid residue in the AMYR agonist polypeptide by a linker, wherein the linker is γE, γE-γE, (O(2Oc)-O(2Oc)-γE), (O(2Oc)-O(2Oc)-γEγE), or ((PEG2)-(PEG2)-γE).

14. The method according to claim 12, wherein the lysine at position 1 of the AMYR agonist polypeptide, or pharmaceutically acceptable salt thereof, is lipidated; wherein the lysine at position 1 of the AMYR agonist polypeptide is lipidated, the lipid is linked to the lysine via a γE-γE linker, and the lipid is octadecanedioic acid (C18diacid).

15. The method according to claim 11, wherein AMYR agonist polypeptide, or pharmaceutically acceptable salt thereof, comprises an amino acid sequence having at least 90%, at least 95%, at least 97%, at least 99%, or 100% identity to an amino acid sequence selected from the group consisting of:
(SEQ ID NO: 6)
C18diacid-γE-[CNTATC]ATQRLAEFLRHSSNN(αMePhe)
GPILPPTNVGSNTY-amide;
(SEQ ID NO: 7)
K(γE-γE-C18diacid)[CNTATC]ATQRLAEFLRHSSNN
(αMePhe)GPILPPTNVGSNTY-amide;
(SEQ ID NO: 8)
K(γE-C18diacid)K[CNTATC]ATQRLAEFLRHSSNN
(αMePhe)GPILPPTNVGSNTY-amide;
(SEQ ID NO: 9)
K(γE-C18diacid)K[CNTATC]ATQRLANFLRHSSNN
(αMePhe)GPILPPTEVGSNTY-amide;
(SEQ ID NO: 10)
K(O2Oc-O2Oc-γE-18diacid)[CNTATC]ATQRLAN
FLRHSSNN(αMePhe)GPILPPTEVGSNTY-amide;
(SEQ ID NO: 11)
K(γE-γE-C18diacid)[CNTATC]ATQRLANFLRHS
SNN(αMePhe)GPILPPTEVGSNTY-amide;
(SEQ ID NO: 12)
K(γE-C18diacid)[CNTATC]ATQRLANFLRHSSNN
(αMePhe)GPILPPTNVGSNTY-amide;
(SEQ ID NO: 13)
K(γE-γE-C18diacid)[CNTATC]ATQRLANFLRHS
SNN(αMePhe)GPILPPTNVGSNTY-amide;
(SEQ ID NO: 14)
K(γE-γE-C18diacid)[CNTATC]ATQRLANFLVHS
SNN(αMePhe)GPILPPTNVGSRTY-amide;
(SEQ ID NO: 15)
K(γE-γE-C18diacid)[CNTATC]ATQRLANFLVHS
SNN(αMePhe)GPILPPTRVGSNTY-amide;
(SEQ ID NO: 16)
K(γE-C18diacid)[CNTATC]ATQRLANFLVHSSNN
(αMePhe)GPILPPTRVGSNTY-amide;
(SEQ ID NO: 17)
K(γE-C18diacid)K[CNTATC]ATQRLA(Dab)
FLVHSSNNFGPILPPTNVGSNTY-amide;
(SEQ ID NO: 18)
K(γE-C18diacid)K[CNTATC]ATQRLANFLVHSS
(Aib)NFGPILPPTHVGSNTY-amide;
(SEQ ID NO: 19)
K(γE-C18diacid)[CNTATC]ATQRLANFLRHSS
(Aib)NFGPILPPTEVGSNTY-amide;
(SEQ ID NO: 20)
K(γE-C18diacid)[CNTATC]ATQRLANFLRHSS
(Aib)NFGPILPPTNVGSNTY-amide;
(SEQ ID NO: 21)
K(γE-γE-C18diacid)K[CNTATC]ATQRLANFLR
HSS(Aib)NFGPILPPTNVGSNTY-amide;
(SEQ ID NO: 22)
K(γE-γE-C18diacid)K[CNTATC]ATQRLANFLVH
SS(Aib)NFGPILPPTRVGSNTY-amide;
(SEQ ID NO: 23)
K(γE-C18diacid)K[CNTATC]ATQRLANFLVHSS
(Aib)NFGPILPPT(Aib)VGSNTY-amide;
(SEQ ID NO: 24)
K(γE-C18diacid)K[CNTATC]ATQRLANFL(Aib)
HSSNNFGPILPPTNVGSNTY-amide;
and
(SEQ ID NO: 25)
K(γE-C18diacid)K[CNTATC]ATQRLA(Dab)FL
(Aib)HSSNNFGPILPPTEVGSNTY-amide.

16. The method according to claim 11, wherein the AMYR agonist polypeptide comprises an amino acid sequence having at least 90%, at least 95%, at least 97%, at least 99%, or 100% identity to the amino acid sequence K[CNTATC]ATQRLANFLRHSSNN(αMePhe)GPILPPTEVGSNTY-amide (SEQ ID NO: 26).

17. The method according to claim 16, wherein the AMYR agonist polypeptide, or pharmaceutically acceptable salt thereof, comprises the amino acid sequence K (γE-γE-C18diacid)[CNTATC]ATQRLANFLRHSSNN(αMePhe)GPILPPTEVGSNTY-amide (SEQ ID NO: 11).

18. The method according to claim 1, wherein the GLP-1R agonist is a GLP-1R agonist polypeptide, or pharmaceutically acceptable salt thereof, which comprises an amino acid sequence having at least 60%, at least 65%, at least 70%, at least 75%, at least 80%, at least 85%, at least 90%, at least 95%, or at least 97% identity to cotadutide (HSQGTFTSD-X10-SEYLDSERARDFVAWLEAGG-acid, wherein X10=Lys[ε-γE-Palmitoyl], SEQ ID NO: 32).

19. The method according to claim 18, wherein the GLP-1R agonist polypeptide, or pharmaceutically acceptable salt thereof, comprises an amino acid sequence having at least 90%, at least 95%, at least 97%, at least 99%, or 100% identity to the amino acid sequence H(Aib)QGTFTSDVSK(αMePhe)LDTKRARDFVQWLLE(Aib)G-acid (SEQ ID NO: 33).

20. The method according to claim 19, wherein the GLP-1R agonist polypeptide, or pharmaceutically acceptable salt thereof, is lipidated, optionally wherein the lipid is selected from the group consisting of C18diacid, and C20diacid.

21. The method according to claim 19, wherein the lysine at position 17 of the GLP-1R agonist polypeptide is lipidated and/or acylated; wherein the lipid is linked to the epsilon amino group of lysine at position 17 of the GLP-1R agonist polypeptide via a linker; wherein the lysine at position 17 of the GLP-1R agonist polypeptide is acylated and lipidated, the lipid is linked to the acylated lysine via its epsilon amino group to (O(2Oc)-(O2Oc)-γE) linker in the C- to N-terminal orientation, and the lipid is octadecanedioic acid (C18diacid).

22. The method according to claim 18, wherein the GLP-1R agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

23. A method for treating and/or preventing a disease or disorder in a subject, the method comprising administering to the subject (a) a polypeptide, or pharmaceutically acceptable salt thereof, which is an AMYR agonist comprising an amino acid sequence having at least 90% identity to pramlintide (KCNTATCATQRLANFLVHSSNNFGPILPPTNVGSNTY-amide, SEQ ID NO: 5); and (b) a GLP-1R agonist.

24. The method according to claim 23, wherein the AMYR agonist polypeptide, or pharmaceutically acceptable salt thereof, comprises the amino acid sequence K(γE-γE-C18diacid)[CNTATC]ATQRLANFLRHSSNN(αMePhe)GPILPPTEVGSNTY-amide (SEQ ID NO: 11).

25. (canceled)

26. A method for treating a disease or disorder in a subject, the method comprising administering to the subject (a) an AMYR agonist; and (b) a polypeptide, or pharmaceutically acceptable salt thereof, which is a GLP-1R agonist polypeptide comprising an amino acid sequence having at least 60%, at least 65%, at least 70%, at least 75%, at least 80%, at least 85%, at least 90%, at least 95%, or at least 97% identity to cotadutide (HSQGTFTSD-X10-SEYLDSERARDFVAWLEAGG-acid, wherein X10=Lys[ε-γE-Palmitoyl], SEQ ID NO: 32); optionally wherein the GLP-1R agonist comprises an amino acid sequence having at least 65% identity to cotadutide.

27. The method according to claim 26, wherein the GLP-1R agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

28. (canceled)

29. The method according to claim 1, wherein the AMYR agonist polypeptide, or pharmaceutically acceptable salt thereof, comprises the amino acid sequence K(γE-γE-C18diacid)[CNTATC]ATQRLANFLRHSSNN(αMePhe)GPILPPTEVGSNTY-amide (SEQ ID NO: 11); and the GLP-1R agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

30. The method of claim 1, wherein the disease or disorder is obesity.

31-35. (canceled)

36. A method of inhibiting or reducing weight gain, promoting weight loss, reducing food intake, increasing satiety, improving glycemic and/or metabolic control, and/or reducing excess body weight in a subject, the method comprising administering the AMYR agonist, or pharmaceutically acceptable salts thereof and the GLP-1R agonist, or pharmaceutically acceptable salts thereof, as defined in claim 4 to the subject; optionally wherein:
(a) the GLP-1R agonist activates both a GLP-1R and a GCGR; optionally wherein the GLP-1R has selectivity to GLP-1R as compared to GCGR; or
(b) the method further comprises administering to the subject a GCGR agonist.

37. (canceled)

38. A method of reducing fat-mass specific body weight in a subject, the method comprising administering the AMYR agonist, or pharmaceutically acceptable salts thereof, and the GLP-1R agonist, or pharmaceutically acceptable salts thereof, as defined in claim 4, to the subject; optionally wherein:
(a) the GLP-1R agonist activates both a GLP-1R and a GCGR; optionally wherein the GLP-1R has selectivity to GLP-1R as compared to GCGR; or
(b) the method further comprises administering to the subject a GCGR agonist.

39-41. (canceled)

42. The method of claim 1, wherein the AMYR agonist and GLP-1R agonist, or pharmaceutically acceptable salts thereof, are administered to the subject orally or by subcutaneous injection.

43-47. (canceled)

48. A method of inhibiting or reducing weight gain, promoting weight loss, reducing food intake, increasing satiety, and/or reducing excess body weight in a subject, the method comprising administering the AMYR agonist, or pharmaceutically acceptable salts thereof as defined in claim 4 to the subject, wherein said subject:
(i) is overweight or obese and has type 2 diabetes; and
(ii) who is being treated with a GLP-1R agonist at the time treatment with the AMYR agonist commences, and optionally who continues treatment with a GLP-1R agonist after treatment with the AMYR agonist commences.

49. A composition comprising the AMYR agonist, or pharmaceutically acceptable salts thereof and the GLP-1R agonist, or pharmaceutically acceptable salts thereof as defined in claim 4.

50. A kit comprising the AMYR agonist, or pharmaceutically acceptable salts thereof and the GLP-1R agonist, or pharmaceutically acceptable salts thereof as defined in claim 4.

51. (canceled)


SEQUENCE LISTING (38 sequences)

SEQ ID NO: 1 - protein, 147 aa, Homo sapiens
ARALCRLPRRGLWLLLAHHLFMTTACQEANYGALLRELCLTQFQVDMEAVGETLWCDWGRTIRSYRELADCTWHMAEKLGCFWPNAEVDRFFLAVHGRYFRSCPISGRAVRDPPGSILYPFIVVPITVTLLVTALVVWQSKRTEGIV

SEQ ID NO: 2 - protein, 174 aa, Homo sapiens
ASLRVERAGGPRLPRTRVGRPAALRLLLLLGAVLNPHEALAQPLPTTGTPGSEGGTVKNYETAVQFCWNHYKDQMDPIEKDWCDWAMISRPYSTLRDCLEHFAELFDLGFPNPLAERIIFETHQIHFANCSLVQPTFSDPPEDVLLAMIIAPICLIPFLITLVVWRSKDSEAQA

SEQ ID NO: 3 - protein, 147 aa, Homo sapiens
ETGALRRPQLLPLLLLLCGGCPRAGGCNETGMLERLPLCGKAFADMMGKVDVWKWCNLSEFIVYYESFTNCTEMEANVVGCYWPNPLAQGFITGIHRQFFSNCTVDRVHLEDPPDEVLIPLIVIPVVLTVAMAGLVVWRSKRTDTLL

SEQ ID NO: 4 - protein, 628 aa, Homo sapiens
GCKVLLNIGQQMLRRKVVDCSREETRLSRCLNTFDLVALGVGSTLGAGVYVLAGAVARENAGPAIVISFLIAALASVLAGLCYGEFGARVPKTGSAYLYSYVTVGELWAFITGWNLILSYIIGTSSVARAWSATFDELIGRPIGEFSRTHMTLNAPGVLAENPDIFAVIIILILTGLLTLGVKESAMVNKIFTCINVLVLGFIMVSGFVKGSVKNWQLTEEDFGNTSGRLCLNNDTKEGKPGVGGFMPFGFSGVLSGAATCFYAFVGFDCIATTGEEVKNPQKAIPVGIVASLLICFIAYFGVSAALTLMMPYFCLDNNSPLPDAFKHVGWEGAKYAVAVGSLCALSASLLGSMFPMPRVIYAMAEDGLLFKFLANVNDRTKTPIIATLASGAVAAVMAFLFDLKDLVDLMSIGTLLAYSLVAACVLVLRYQPEQPNLVYQMASTSDELDPADQNELASTNDSQLGFLPEAEMFSLKTILSPKNMEPSKISGLIVNISTSLIAVLIITFCIVTVLGREALTKGALWAVFLLAGSALLCAVVTGVIWRQPESKTKLSFKVPFLPVLPILSIFVNVYLMMQLDQGTWVRFAVWMLIGFIIYFGYGLWHSEEASLDADQARTPDGNLDQCK

SEQ ID NO: 5 - protein, 37 aa, synthetic construct
  SITE 37: note = amide C-terminus
  DISULFID 2..7: note = disulfid bond
KCNTATCATQRLANFLVHSSNNFGPILPPTNVGSNTY

SEQ ID NO: 6 - protein, 37 aa, synthetic construct
  DISULFID 2..7: note = disulfide bond
  SITE 1: note = yE-C18diacid
  SITE 23: note = (S)-2-amino-2-methyl-3-phenylpropanoic acid
  SITE 37: note = amide C-terminus
XCNTATCATQRLAEFLRHSSNNFGPILPPTNVGSNTY

SEQ ID NO: 7 - protein, 37 aa, synthetic construct
  DISULFID 2..7: note = disulfid bond
  SITE 1: note = side chain of Lys is connected to yE-yE-C18diacid
  SITE 37: note = amide C-terminus
  SITE 23: note = (S)-2-amino-2-methyl-3-phenylpropanoic acid
KCNTATCATQRLAEFLRHSSNNFGPILPPTNVGSNTY

SEQ ID NO: 8 - protein, 38 aa, synthetic construct
  DISULFID 3..8: note = disulfid bond
  SITE 1: note = side chain of Lys is connected to yE-C18diacid
  SITE 38: note = amide C-terminus
  SITE 24: note = (S)-2-amino-2-methyl-3-phenylpropanoic acid
KKCNTATCATQRLAEFLRHSSNNFGPILPPTNVGSNTY

SEQ ID NO: 9 - protein, 38 aa, synthetic construct
  DISULFID 3..8: note = disulfid bond
  SITE 1: note = side chain of Lys is connected to yE-C18diacid
  SITE 38: note = amide C-terminus
  SITE 24: note = (S)-2-amino-2-methyl-3-phenylpropanoic acid
KKCNTATCATQRLANFLRHSSNNFGPILPPTEVGSNTY

SEQ ID NO: 10 - protein, 37 aa, synthetic construct
  DISULFID 2..7: note = disulfid bond
  SITE 1: note = side chain of Lys is connected to O2Oc-O2Oc-yE-C18diacid
  SITE 23: note = (S)-2-amino-2-methyl-3-phenylpropanoic acid
  SITE 37: note = amide C-terminus
KCNTATCATQRLANFLRHSSNNFGPILPPTEVGSNTY

SEQ ID NO: 11 - protein, 37 aa, synthetic construct; identical to SEQ ID NO: 10
  DISULFID 2..7: note = disulfide bond
  SITE 1: note = side chain of Lys is connected to yE-yE-C18diacid
  SITE 23: note = (S)-2-amino-2-methyl-3-phenylpropanoic acid
  SITE 37: note = amide C-terminus

SEQ ID NO: 12 - protein, 37 aa, synthetic construct
  DISULFID 2..7: note = disulfid bond
  SITE 1: note = side chain of Lys is connected to yE-C18diacid
  SITE 23: note = (S)-2-amino-2-methyl-3-phenylpropanoic acid
  SITE 37: note = amide C-terminus
KCNTATCATQRLANFLRHSSNNFGPILPPTNVGSNTY

SEQ ID NO: 13 - protein, 37 aa, synthetic construct; identical to SEQ ID NO: 12
  DISULFID 2..7: note = disulfid bond
  SITE 1: note = side chain of Lys is connected to yE-yE-C18diacid
  SITE 23: note = (S)-2-amino-2-methyl-3-phenylpropanoic acid
  SITE 37: note = amide C-terminus

SEQ ID NO: 14 - protein, 37 aa, synthetic construct
  DISULFID 2..7: note = disulfid bond
  SITE 1: note = side chain of Lys is connected to yE-yE-C18diacid
  SITE 23: note = (S)-2-amino-2-methyl-3-phenylpropanoic acid
  SITE 37: note = amide C-terminus
KCNTATCATQRLANFLVHSSNNFGPILPPTNVGSRTY

SEQ ID NO: 15 - protein, 37 aa, synthetic construct
  DISULFID 2..7: note = disulfid bond
  SITE 1: note = side chain of Lys is connected to yE-yE-C18diacid
  SITE 23: note = (S)-2-amino-2-methyl-3-phenylpropanoic acid
  SITE 37: note = amide C-terminus
KCNTATCATQRLANFLVHSSNNFGPILPPTRVGSNTY

SEQ ID NO: 16 - protein, 37 aa, synthetic construct; identical to SEQ ID NO: 15
  DISULFID 2..7: note = disulfid bond
  SITE 1: note = side chain of Lys is connected to yE-C18diacid
  SITE 23: note = (S)-2-amino-2-methyl-3-phenylpropanoic acid
  SITE 37: note = amide C-terminus

SEQ ID NO: 17 - protein, 38 aa, synthetic construct
  DISULFID 3..8: note = disulfid bond
  SITE 1: note = side chain of Lys is connected to yE-C18diacid
  SITE 15: note = Dbu
  SITE 38: note = amide C-terminus
KKCNTATCATQRLAXFLVHSSNNFGPILPPTNVGSNTY

SEQ ID NO: 18 - protein, 38 aa, synthetic construct
  DISULFID 3..8: note = disulfid bond
  SITE 1: note = side chain of Lys is connected to yE-C18diacid
  SITE 22: note = Aib
  SITE 38: note = amide C-terminus
KKCNTATCATQRLANFLVHSSXNFGPILPPTHVGSNTY

SEQ ID NO: 19 - protein, 37 aa, synthetic construct
  DISULFID 2..7: note = disulfid bond
  SITE 1: note = side chain of Lys is connected to yE-C18diacid
  SITE 21: note = Aib
  SITE 37: note = amide C-terminus
KCNTATCATQRLANFLRHSSXNFGPILPPTEVGSNTY

SEQ ID NO: 20 - protein, 37 aa, synthetic construct
  DISULFID 2..7: note = disulfid bond
  SITE 1: note = side chain of Lys is connected to yE-C18diacid
  SITE 21: note = Aib
  SITE 37: note = amide C-terminus
KCNTATCATQRLANFLRHSSXNFGPILPPTNVGSNTY

SEQ ID NO: 21 - protein, 38 aa, synthetic construct
  DISULFID 3..8: note = disulfid bond
  SITE 1: note = side chain of Lys is connected to yE-yE-C18diacid
  SITE 22: note = Aib
  SITE 38: note = amide C-terminus
KKCNTATCATQRLANFLRHSSXNFGPILPPTNVGSNTY

SEQ ID NO: 22 - protein, 38 aa, synthetic construct
  DISULFID 3..8: note = disulfid bond
  SITE 1: note = side chain of Lys is connected to yE-yE-C18diacid
  SITE 22: note = Aib
  SITE 38: note = amide C-terminus
KKCNTATCATQRLANFLVHSSXNFGPILPPTRVGSNTY

SEQ ID NO: 23 - protein, 38 aa, synthetic construct
  DISULFID 3..8: note = disulfid bond
  SITE 1: note = side chain of Lys is connected to yE-C18diacid
  SITE 22: note = Aib
  SITE 32: note = Aib
  SITE 38: note = amide C-terminus
KKCNTATCATQRLANFLVHSSXNFGPILPPTXVGSNTY

SEQ ID NO: 24 - protein, 38 aa, synthetic construct
  DISULFID 3..8: note = disulfid bond
  SITE 1: note = side chain of Lys is connected to yE-C18diacid
  SITE 18: note = Aib
  SITE 38: note = amide C-terminus
KKCNTATCATQRLANFLXHSSNNFGPILPPTNVGSNTY

SEQ ID NO: 25 - protein, 38 aa, synthetic construct
  DISULFID 3..8: note = disulfid bond
  SITE 1: note = side chain of Lys is connected to yE-C18diacid
  SITE 15: note = Dbu
  SITE 18: note = Aib
  SITE 38: note = amide C-terminus
KKCNTATCATQRLAXFLXHSSNNFGPILPPTEVGSNTY

SEQ ID NO: 26 - protein, 37 aa, synthetic construct; identical to SEQ ID NO: 10
  DISULFID 2..7: note = disulfid bond
  SITE 23: note = (S)-2-amino-2-methyl-3-phenylpropanoic acid
  SITE 37: note = amide C-terminus

SEQ ID NO: 27 - protein, 30 aa, Homo sapiens
  SITE 30: note = amide C-terminus
HAEGTFTSDVSSYLEGQAAKEFIAWLVKGR

SEQ ID NO: 28 - protein, 31 aa, Homo sapiens
HAEGTFTSDVSSYLEGQAAKEFIAWLVKGRG

SEQ ID NO: 29 - protein, 462 aa, Homo sapiens
AGAPGLLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 30 - protein, 179 aa, Homo sapiens
KSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 31 - protein, 476 aa, Homo sapiens
PPCQPQRPLLLLLLLLACQPQVPSAQVMDFLFEKWKLYGDQCHHNLSLLPPPTELVCNRTFDKYSCWPDTPANTTANISCPWYLPWHHKVQHRFVFKRCGPDGQWVRGPRGQPWRDASQCQMDGEEIEVQKEVAKMYSSFQVMYTVGYSLSLGALLLALAILGGLSKLHCTRNAIHANLFASFVLKASSVLVIDGLLRTRYSQKIGDDLSVSTWLSDGAVAGCRVAAVFMQYGIVANYCWLLVEGLYLHNLLGLATLPERSFFSLYLGIGWGAPMLFVVPWAVVKCLFENVQCWTSNDNMGFWWILRFPVFLAILINFFIFVRIVQLLVAKLRARQMHHTDYKFRLAKSTLTLIPLLGVHEVVFAFVTDEHAQGTLRSAKLFFDLFLSSFQGLLVAVLYCFLNKEVQSELRRRWHRWRLGKVLWEERNTSNHRASSSPGHGPPSKELQFGRGGGSQDSSAETPLAGGLPRLAESPF

SEQ ID NO: 32 - protein, 30 aa, synthetic construct
  SITE 10: note = side chain of Lys is connected to yE-Palmitoyl
HSQGTFTSDKSEYLDSERARDFVAWLEAGG

SEQ ID NO: 33 - protein, 30 aa, synthetic construct
  SITE 2: note = Aib
  SITE 13: note = (S)-2-amino-2-methyl-3-phenylpropanoic acid
  SITE 29: note = Aib
HXQGTFTSDVSKFLDTKRARDFVQWLLEXG

SEQ ID NO: 34 - protein, 29 aa, synthetic construct
  SITE 2: note = Aib
  SITE 6: note = (S)-2-amino-2-methyl-3-phenylpropanoic acid
  SITE 13: note = (S)-2-amino-2-methyl-3-phenylpropanoic acid
  SITE 22: note = (S)-2-amino-2-methyl-3-phenylpropanoic acid
  SITE 25: note = Aib
  SITE 29: note = amide C-terminus
HXHGSFTSDVSKFLDSRAAKDFVQXIANT

SEQ ID NO: 35 - protein, 30 aa, synthetic construct; identical to SEQ ID NO: 33
  SITE 2: note = Aib
  SITE 29: note = Aib
  SITE 13: note = (S)-2-amino-2-methyl-3-phenylpropanoic acid
  SITE 17: note = side chain of Lys is connected to O2Oc-O2Oc-yE-C18diacid

SEQ ID NO: 36 - protein, 29 aa, synthetic construct
  SITE 2: note = Aib
  SITE 6: note = (S)-2-amino-2-methyl-3-phenylpropanoic acid
  SITE 13: note = (S)-2-amino-2-methyl-3-phenylpropanoic acid
  SITE 20: note = side chain of Lys is connected to O2Oc-O2Oc-yE-C18diacid
  SITE 22: note = (S)-2-amino-2-methyl-3-phenylpropanoic acid
  SITE 25: note = Aib
  SITE 29: note = amide C-terminus
HXHGSFTSDVSKFLDSRAAKDFVQXAINT

SEQ ID NO: 37 - protein, 30 aa, synthetic construct; identical to SEQ ID NO: 33
  SITE 2: note = Aib
  SITE 13: note = (S)-2-amino-2-methyl-3-phenylpropanoic acid
  SITE 17: note = side chain of Lys is connected to O2Oc-O2Oc-yE-C20diacid
  SITE 29: note = Aib

SEQ ID NO: 38 - protein, 29 aa, synthetic construct
  SITE 2: note = Aib
  SITE 6: note = (S)-2-amino-2-methyl-3-phenylpropanoic acid
  SITE 13: note = (S)-2-amino-2-methyl-3-phenylpropanoic acid
  SITE 20: note = side chain of Lys is connected to O2Oc-O2Oc-yE-yE-C20diacid
  SITE 22: note = (S)-2-amino-2-methyl-3-phenylpropanoic acid
  SITE 25: note = Aib
  SITE 29: note = amide C-terminus
HXHGSFTSDVSKFLDSRAAKDFVQXIANT